SEQUENCE LISTING (2 sequences)

SEQ ID NO: 1 - protein, 30 aa, synthetic construct
  REGION 1..30: note = Synthetic
MQIPQAPWPVVWAVLQLGWRPGWFLDSPDR

SEQ ID NO: 2 - protein, 23 aa, synthetic construct
  REGION 1..23: note = Synthetic
MRANDALQVLGLLFSLARGSEVG

(19) United States
(12) Patent Application Publication
(10) Pub. No.: US 2025/0171434 A1
(43) Pub. Date: May 29, 2025

(54) SEC61 INHIBITORS AND USE THEREOF

(71) Applicant: KEZAR LIFE SCIENCES, INC., South San Francisco, CA (US)

(72) Inventors: Dustin McMinn, Pacifica, CA (US); Meera Rao, San Francisco, CA (US); Chengguo Dong, South San Francisco, CA (US); Neel Kumar Anand, South San Francisco, CA (US)

(21) Appl. No.: 18/841,805

(22) PCT Filed: Feb. 28, 2023
(86) PCT No.: PCT/US2023/014019

§ 371 Date: Aug. 27, 2024

Related U.S. Application Data
(60) Provisional application No. 63/314,554, filed on Feb. 28, 2022.

(51) Int. Cl. C07D 417/14; A61K 31/4439; A61K 31/444; A61K 31/519; C07D 487/04

(52) U.S. Cl. CPC C07D 417/14; A61K 31/4439; A61K 31/444; A61K 31/519; C07D 487/04

(57) ABSTRACT

Provided herein are secretion inhibitors, such as inhibitors of Sec61 having a structure of formula (I): methods for their preparation, related pharmaceutical compositions, and methods for using the same.

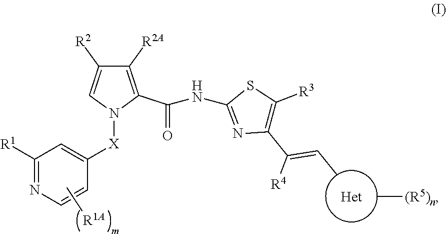

### BACKGROUND

### Field of the Invention

The present disclosure relates to protein secretion inhibitors, including methods of making and using the same.

### INCORPORATION BY REFERENCE OF MATERIAL SUBMITTED ELECTRONICALLY

The Sequence Listing, which is a part of the present disclosure, is submitted concurrently with the specification. The name of the file containing the Sequence Listing is 40066_Seqlisting.xml, which was created on Jan. 13, 2023. The subject matter of the Sequence Listing is incorporated herein in its entirety by reference.

### Description of Related Technology

Protein translocation into the endoplasmic reticulum (“ER”) constitutes the first step of protein secretion. ER protein import is essential in all eukaryotic cells and is particularly important in fast-growing tumor cells. Thus, the process of protein secretion can serve as a target both for potential cancer drugs and for bacterial virulence factors. See Kalies and Römisch, Traffic, 16(10):1027-1038 (2015).

Protein transport to the ER is initiated in the cytosol when N-terminal hydrophobic signal peptides protrude from the ribosome. Binding of signal recognition particle (“SRP”) to the signal sequence allows targeting of the ribosome-nascent chain-SRP complex to the ER membrane where contact of SRP with its receptor triggers handing over of the signal peptide to Sec61. Sec61 is an ER membrane protein translocator (aka translocon) that is doughnut-shaped with 3 major subunits (heterotrimeric). It includes a “plug,” which blocks transport into or out of the ER. The plug is displaced when the hydrophobic region of a nascent polypeptide interacts with the “seam” region of Sec61, allowing translocation of the polypeptide into the ER lumen. In mammals, only short proteins (<160 amino acids) can enter the ER posttranslationally, and proteins smaller than 120 amino acids are obliged to use this pathway. Some of the translocation competence is maintained by the binding of calmodulin to the signal sequence. Upon arrival at the Sec61 channel, the signal peptide or signal anchor intercalates between transmembrane domains (“TMDs”) 2 and 7 of Sec61α, which form the lateral portion of the gate, allowing the channel to open for soluble secretory proteins. As the Sec61 channel consists of 10 TMDs (Sec61α) surrounded by a hydrophobic clamp formed by Sec61γ, channel opening is dependent on conformational changes that involve practically all TMDs.

Inhibition of protein transport across the ER membrane has the potential to treat or prevent diseases, such as the growth of cancer cells and inflammation. Known secretion inhibitors, which range from broad-spectrum to highly substrate-specific, can interfere with virtually any stage of this multistep process, and even with transport of endocytosed antigens into the cytosol for cross-presentation. These inhibitors interact with the signal peptide, chaperones, or the Sec61 channel to block substrate binding or to prevent the conformational changes needed for protein import into the ER. Examples of protein secretion inhibitors include, calmodulin inhibitors (e.g., E6 Berbamine and Ophiobolin A), Lanthanum, sterols, cyclodepsipeptides (e.g., HUN-7293, CAM741, NFI028, Cotrainsin, Apratoxin A, Decatransin, Valinomycin), CADA, Mycolactone, Eeyarestatin I (“ESI”), and Exotoxin A. However, the above secretion inhibitors suffer from one or more of the following: lack selectivity for the Sec61 channel, challenging manufacture due to structural complexity, and molecular weight limited administration, bio-availability and distribution.

Thus, a need exits for new inhibitors of protein secretion.

### SUMMARY

Provided herein are compounds, or salts thereof, having a structure of formula (I):

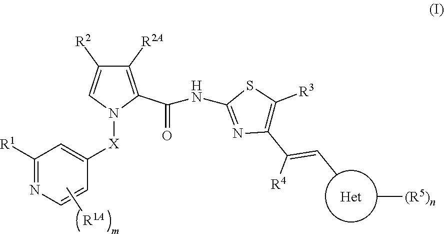

where the substituents are as disclosed below.

Also provided are pharmaceutical compositions comprising the compound or salt described herein and a pharmaceutically acceptable carrier.

Further provided are methods of inhibiting protein secretion in a cell comprising contacting the cell with the compound, salt, or pharmaceutical composition described herein in an amount effective to inhibit secretion. In some embodiments, the protein is a checkpoint protein. In some embodiments, the protein is a cell-surface protein, endoplasmic reticulum associated protein, or secreted protein involved in regulation of anti-tumor immune response. In various cases, the protein is at least one of PD-1, PD-L1, TIM-1, LAG-3, CTLA4, BTLA, OX-40, B7H1, B7H4, CD137, CD47, CD96, CD73, CD40, VISTA, TIGIT, LAIR1, CD160, 2B4, TGFRβ and combinations thereof. In some cases, the protein is selected from the group consisting of HER3, TNFα, IL2, and PD1. In some embodiments, the contacting comprises administering the compound or the composition to a subject in need thereof.

The disclosure also provides methods for treating inflammation in a subject comprising administering to the subject a therapeutically effective amount of the compound, salt, or pharmaceutical composition described herein.

The disclosure further provides methods for treating cancer in a subject comprising administering to the subject a therapeutically effective amount of the compound, salt, or pharmaceutical composition described herein. In some embodiments, the cancer is melanoma, multiple myeloma, prostate cancer, lung cancer, pancreatic cancer, squamous cell carcinoma, leukemia, lymphoma, a neuroendocrine tumor, bladder cancer, or colorectal cancer. In some cases, the cancer is selected from the group consisting of prostate, lung, bladder, colorectal, and multiple myeloma. In some cases, the cancer is non-small cell lung carcinoma, squamous cell carcinoma, leukemia, acute myelogenous leukemia, chronic myelogenous leukemia, lymphoma, NPM/ALK-transformed anaplastic large cell lymphoma, diffuse large B cell lymphoma, neuroendocrine tumors, breast cancer, mantle cell lymphoma, renal cell carcinoma, rhabdomyosarcoma, ovarian cancer, endometrial cancer, small cell carcinoma, adenocarcinoma, gastric carcinoma, hepatocellular carcinoma, pancreatic cancer, thyroid carcinoma, anaplastic large cell lymphoma, hemangioma, or head and neck cancer. In various cases, the cancer is a solid tumor.

Further provided are methods for treating an autoimmune disease in a subject comprising administering to the subject a therapeutically effective amount of the compound, salt, or pharmaceutical composition described herein. In some embodiments, the autoimmune disease is psoriasis, dermatitis, systemic scleroderma, sclerosis, Crohn's disease, ulcerative colitis; respiratory distress syndrome, meningitis; encephalitis; uveitis; colitis; glomerulonephritis; eczema, asthma, chronic inflammation; atherosclerosis; leukocyte adhesion deficiency; rheumatoid arthritis; systemic lupus erythematosus (SLE); diabetes mellitus; multiple sclerosis; Reynaud's syndrome; autoimmune thyroiditis; allergic encephalomyelitis; Sjorgen's syndrome; juvenile onset diabetes; tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

The disclosure also provides methods for the treatment of an immune-related disease in a subject comprising administering to the subject a therapeutically effective amount of the compound, salt, or pharmaceutical composition described herein. In some embodiments, the immune-related disease is rheumatoid arthritis, lupus, inflammatory bowel disease, multiple sclerosis, or Crohn's disease.

Further provided are methods for treating neurodegenerative disease in a subject comprising administering to the subject a therapeutically effective amount of the compound, salt, or pharmaceutical composition described herein. In some cases, the neurodegenerative disease is multiple sclerosis.

Also provided are methods for treating an inflammatory disease in a subject comprising administering to the subject a therapeutically effective amount of the compound, salt, or pharmaceutical composition described herein. In some embodiments, the inflammatory disease is bronchitis, conjunctivitis, myocarditis, pancreatitis, chronic cholecstitis, bronchiectasis, aortic valve stenosis, restenosis, psoriasis or arthritis.

Further aspects and advantages will be apparent to those of ordinary skill in the art from a review of the following detailed description. The description hereafter includes specific embodiments with the understanding that the disclosure is illustrative and is not intended to limit the disclosure to the specific embodiments described herein.

### DETAILED DESCRIPTION

Provided herein are compounds that inhibit protein secretion. The compounds described herein can be used to treat or prevent diseases associated with excessive protein secretion, such as inflammation and cancer, improving the quality of life for afflicted individuals.

### Compounds of Formula (I)

Provided herein are compounds, or pharmaceutically acceptable salts thereof, having a structure of formula (I):

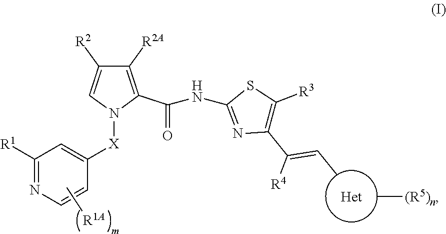

R¹ is H or F;
m is 0, 1, or 2;
each R^{1A} is independently F or C₁₋₃alkyl;
X is CH₂ or CD₂;
one of R² and R^{2A} is halo, CN, OH, C₁₋₃haloalkyl, C₁₋₃alkoxy, C₁₋₃haloalkoxy, —S—C₁₋₃alkyl, or —S—C₁₋₃ haloalkyl, and the other is H;
R³ is H, halo, or OCH₃;
R⁴ is H, D, CH₃, or halo;
each R⁵ is independently selected from D, C₁₋₃alkyl, cyclopropyl, cyano-substituted cyclopropyl, O-cyclopropyl, C₁₋₃haloalkyl, C₁₋₃haloalkoxy, CH₂OH, halo, oxo, CO₂R^{N}, C₀₋₂alkylene-C(O)N(R^{N})₂, N(R^{N})₂, N(R^{N})C(O)R^{N}, and CN;
Het is imidazolyl, oxazolyl, triazolyl, pyridiyl, cyclohexyl, tetrahydrofuranyl, 5, 6, 7, 8-tetrahydroimidazo[1,2,a]pyridyl, or 5, 6, 7, 8-tetrahydroimidazo[1,2,a]pyrimidinyl;
n is 0, 1 or 2, with the proviso that when Het is imidazolyl, then (a) n is 1 and R⁵ is not cyclopropyl, or (b) n is 2 and either (i) at least one R⁵ is halo, CN, C₁₋₃haloalkyl, or C₁₋₃haloalkoxy, or (ii) each R⁵ is C₁₋₃alkyl; and
each R^{N} is independently H or C₁₋₆alkyl, or two R^{N} on the same nitrogen atom, together with the nitrogen to which they are attached, form a 4-7 membered ring having 0-2 additional ring heteroatoms independently selected from N, O, and S.

In various cases, R¹ is F. In various cases, m is 0.

In various cases, m is 1 or 2, and each R^{1A} is independently F or C₁₋₃alkyl.

In various cases, R² or R^{2A} is halo, CN, C₁₋₃alkoxy, C₁₋₃haloalkoxy, and the other is H. In various cases, R² is halo, CN, C₁₋₃alkoxy, C₁₋₃haloalkoxy, F, Cl, CN, OCH₃, or OCHF₂. In various cases, R^{2A} is H. In various cases, R^{2A} is halo, CN, C₁₋₃alkoxy, C₁₋₃haloalkoxy, F, Cl, CN, OCH₃, or OCHF₂. In various uses, R² is OH, C₁₋₃haloalkyl, —S—C₁₋₃alkyl, or —S—C₁₋₃haloalkyl, and R^{2A} is H.

In various cases, R³ is H, halo, or OCH₃. In various cases, R³ is H. In some cases, R³ is halo or OCH₃. In various cases, R⁴ is H, CH₃, or halo. In various cases, X is CH₂. In some cases, X is CD₂. In various cases, n is 0. In various cases, n is 1. In various cases, n is 2. In some cases, at least one R⁵ is halo. In some cases, at least one R⁵ is C₁₋₃alkyl, cyano, cyclopropyl, cyano-substituted cyclopropyl, O-cyclopropyl, C₁₋₃haloalkyl, C₁₋₃ haloalkoxy, or CH₂OH.

In various cases, R^{N} is H or C₁₋₆alkyl, or two R^{N} on the same nitrogen atom, together with the nitrogen to which they are attached, form a 4-7 membered ring having 0-2 additional ring heteroatoms independently selected from N, O, and S.

In various cases, Het is imidizolyl. In some cases, Het is triazolyl, cyclohexyl tetrahydrofuranyl, oxazolyl, or pyridyl. In some cases, Het is 5, 6, 7, 8-tetrahydroimidazo[1,2,a]pyridyl or 5, 6, 7, 8-tetrahydroimidazo[1,2,a]pyrimidinyl.

In various cases,

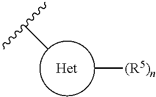

is selected from the group consisting of

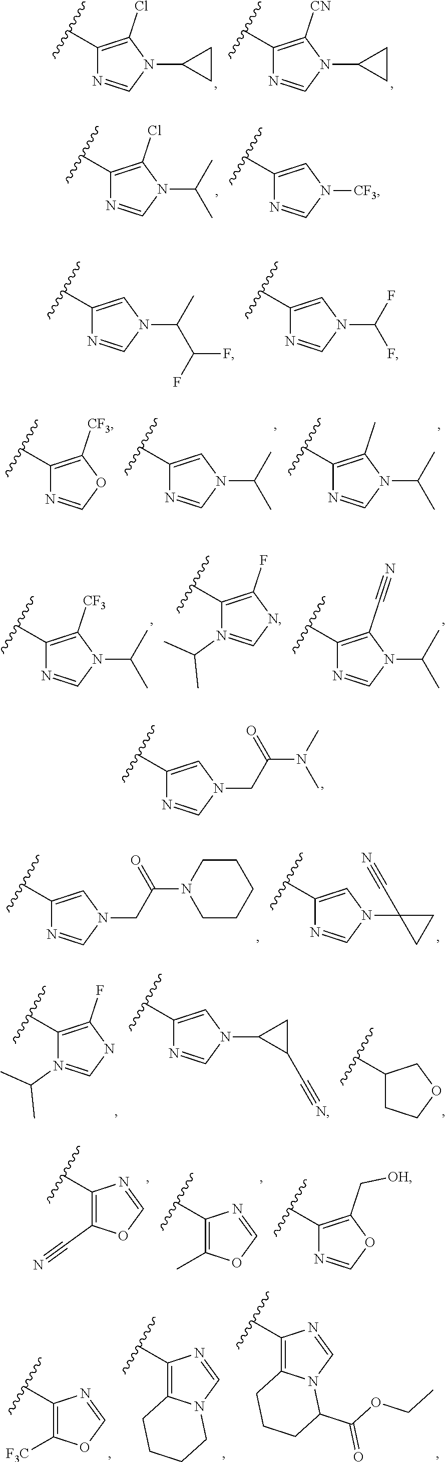

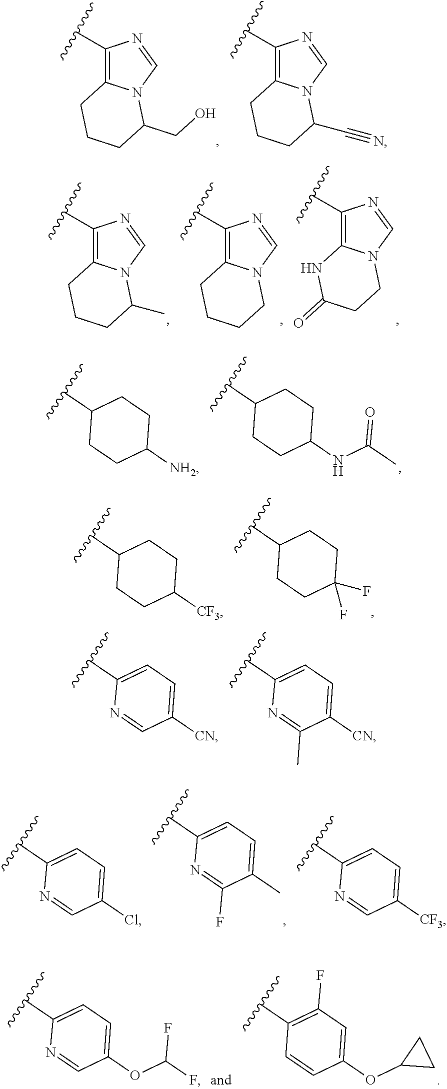

In various cases, the compound of Formula (I), or a compound as disclosed herein, is a structure as shown in Table A or Table B or a pharmaceutically acceptable salt thereof.

TABLE A

| | |
|---|---|
| A1 | 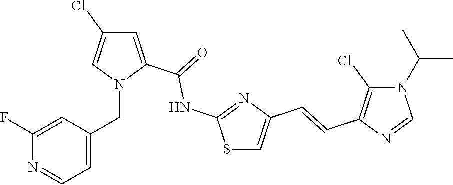 [SMILES: CC(C)n1cnc(/C=C/c2csc(NC(=O)c3cc(Cl)cn3Cc3ccnc(F)c3)n2)c1Cl] |
| A2 | 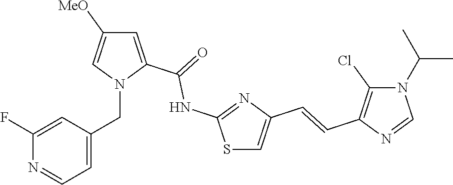 [SMILES: COc1cc(C(=O)Nc2nc(/C=C/c3ncn(C(C)C)c3Cl)cs2)n(Cc2ccnc(F)c2)c1] |
| A3 | 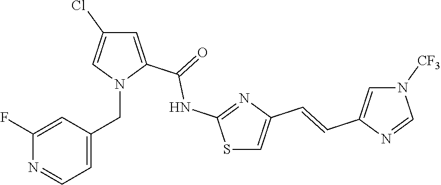 [SMILES: O=C(Nc1nc(/C=C/c2cn(C(F)(F)F)cn2)cs1)c1cc(Cl)cn1Cc1ccnc(F)c1] |
| A4 | 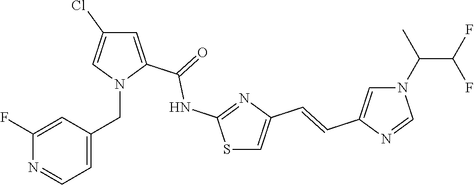 [SMILES: CC(C(F)F)n1cnc(/C=C/c2csc(NC(=O)c3cc(Cl)cn3Cc3ccnc(F)c3)n2)c1] |
| A5 | 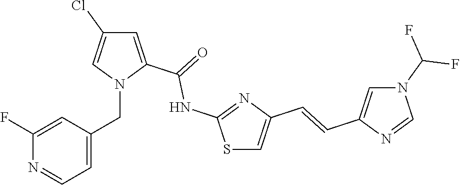 [SMILES: O=C(Nc1nc(/C=C/c2cn(C(F)F)cn2)cs1)c1cc(Cl)cn1Cc1ccnc(F)c1] |
| A6 | 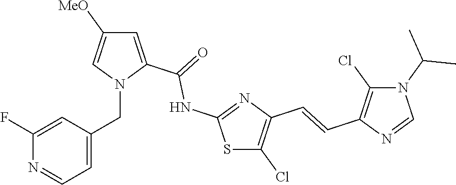 [SMILES: COc1cc(C(=O)Nc2nc(/C=C/c3ncn(C(C)C)c3Cl)c(Cl)s2)n(Cc2ccnc(F)c2)c1] |
| A7 | 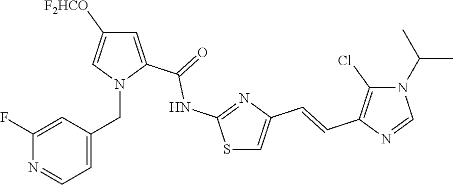 [SMILES: CC(C)n1cnc(/C=C/c2csc(NC(=O)c3cc(OC(F)F)cn3Cc3ccnc(F)c3)n2)c1Cl] |
| A8 | 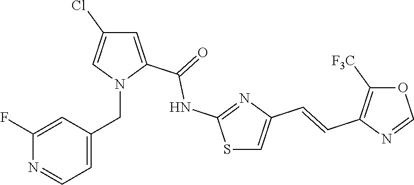 [SMILES: O=C(Nc1nc(/C=C/c2ncoc2C(F)(F)F)cs1)c1cc(Cl)cn1Cc1ccnc(F)c1] |
| A9 | 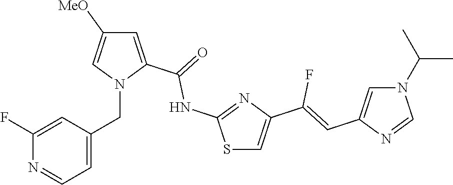 [SMILES: COc1cc(C(=O)Nc2nc(/C(F)=C/c3cn(C(C)C)cn3)cs2)n(Cc2ccnc(F)c2)c1] |
| A10 | 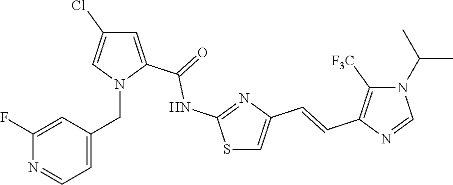 [SMILES: CC(C)n1cnc(/C=C/c2csc(NC(=O)c3cc(Cl)cn3Cc3ccnc(F)c3)n2)c1C(F)(F)F] |
| A11 | 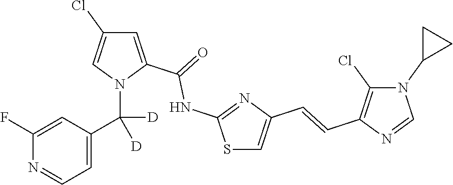 [SMILES: [2H]C([2H])(c1ccnc(F)c1)n1cc(Cl)cc1C(=O)Nc1nc(/C=C/c2ncn(C3CC3)c2Cl)cs1] |
| A12 | 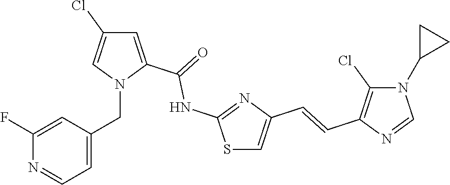 [SMILES: O=C(Nc1nc(/C=C/c2ncn(C3CC3)c2Cl)cs1)c1cc(Cl)cn1Cc1ccnc(F)c1] |
| A13 | 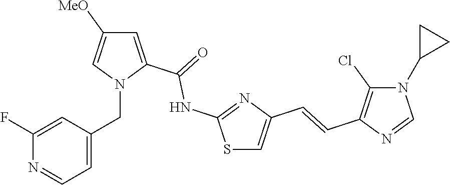 [SMILES: COc1cc(C(=O)Nc2nc(/C=C/c3ncn(C4CC4)c3Cl)cs2)n(Cc2ccnc(F)c2)c1] |
| A14 | 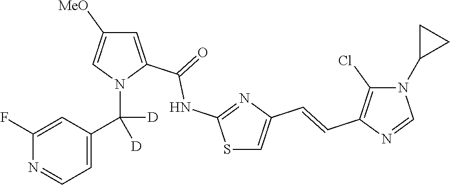 [SMILES: [2H]C([2H])(c1ccnc(F)c1)n1cc(OC)cc1C(=O)Nc1nc(/C=C/c2ncn(C3CC3)c2Cl)cs1] |
| A15 | 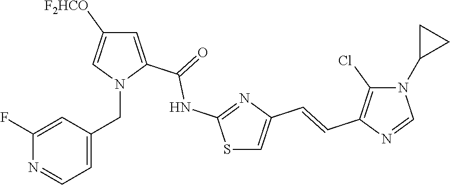 [SMILES: O=C(Nc1nc(/C=C/c2ncn(C3CC3)c2Cl)cs1)c1cc(OC(F)F)cn1Cc1ccnc(F)c1] |
| A16 | 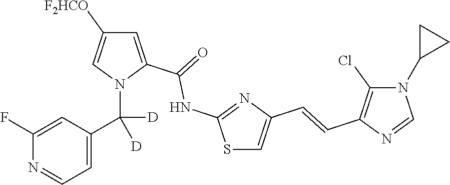 [SMILES: [2H]C([2H])(c1ccnc(F)c1)n1cc(OC(F)F)cc1C(=O)Nc1nc(/C=C/c2ncn(C3CC3)c2Cl)cs1] |
| A17 | 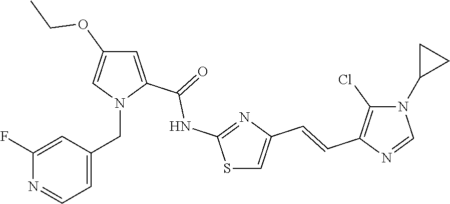 [SMILES: CCOc1cc(C(=O)Nc2nc(/C=C/c3ncn(C4CC4)c3Cl)cs2)n(Cc2ccnc(F)c2)c1] |
| A18 | 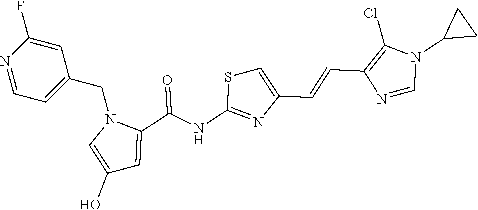 [SMILES: O=C(Nc1nc(/C=C/c2ncn(C3CC3)c2Cl)cs1)c1cc(O)cn1Cc1ccnc(F)c1] |
| A19 | 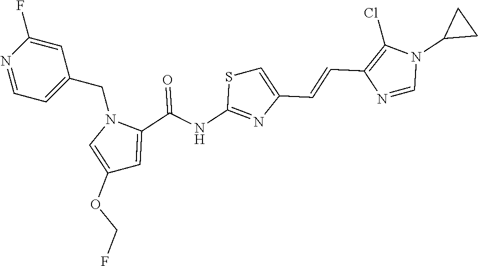 [SMILES: O=C(Nc1nc(/C=C/c2ncn(C3CC3)c2Cl)cs1)c1cc(OCF)cn1Cc1ccnc(F)c1] |
| A20 | 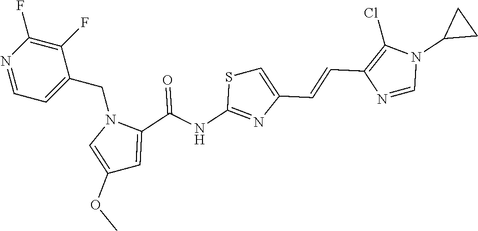 [SMILES: COc1cc(C(=O)Nc2nc(/C=C/c3ncn(C4CC4)c3Cl)cs2)n(Cc2ccnc(F)c2F)c1] |
| A21 | 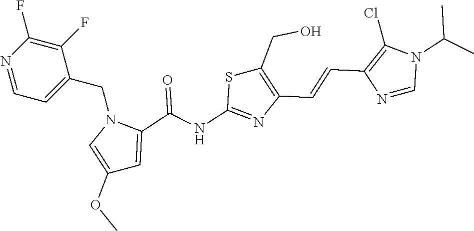 [SMILES: COc1cc(C(=O)Nc2nc(/C=C/c3ncn(C(C)C)c3Cl)c(CO)s2)n(Cc2ccnc(F)c2F)c1] |
| A22 | 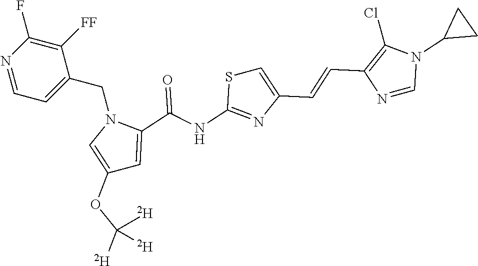 |
| A23 | 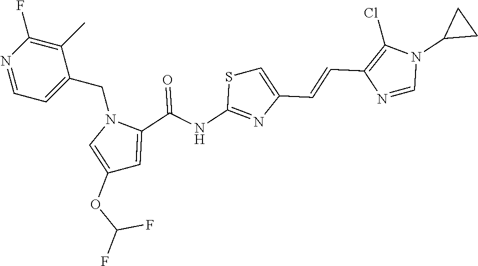 [SMILES: Cc1c(Cn2cc(OC(F)F)cc2C(=O)Nc2nc(/C=C/c3ncn(C4CC4)c3Cl)cs2)ccnc1F] |
| A24 | 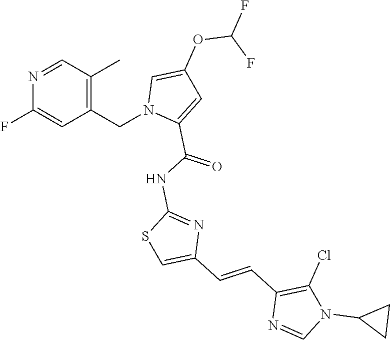 [SMILES: Cc1cnc(F)cc1Cn1cc(OC(F)F)cc1C(=O)Nc1nc(/C=C/c2ncn(C3CC3)c2Cl)cs1] |
| A26 | 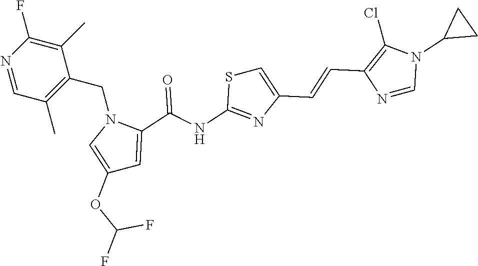 [SMILES: Cc1cnc(F)c(C)c1Cn1cc(OC(F)F)cc1C(=O)Nc1nc(/C=C/c2ncn(C3CC3)c2Cl)cs1] |
| A27 | 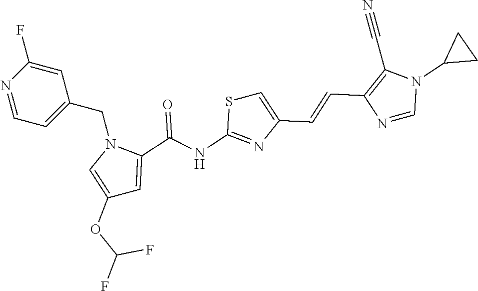 [SMILES: N#Cc1c(/C=C/c2csc(NC(=O)c3cc(OC(F)F)cn3Cc3ccnc(F)c3)n2)ncn1C1CC1] |
| A28 | 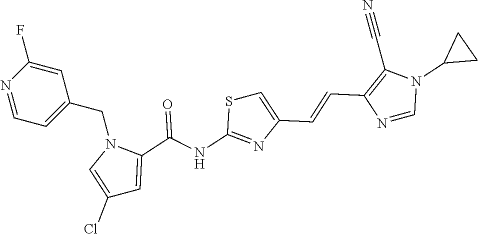 [SMILES: N#Cc1c(/C=C/c2csc(NC(=O)c3cc(Cl)cn3Cc3ccnc(F)c3)n2)ncn1C1CC1] |
| A29 | 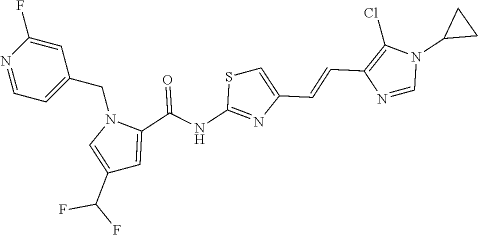 [SMILES: O=C(Nc1nc(/C=C/c2ncn(C3CC3)c2Cl)cs1)c1cc(C(F)F)cn1Cc1ccnc(F)c1] |
| A30 | 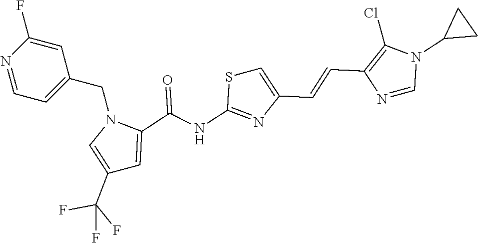 [SMILES: O=C(Nc1nc(/C=C/c2ncn(C3CC3)c2Cl)cs1)c1cc(C(F)(F)F)cn1Cc1ccnc(F)c1] |
| A31 | 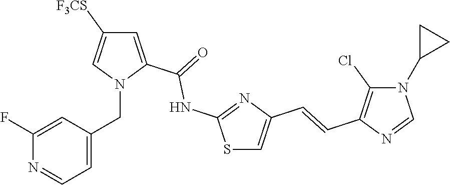 |
| A32 | 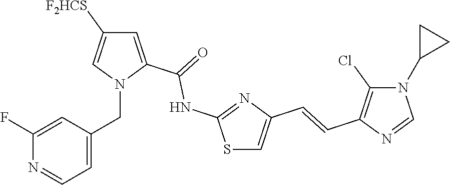 |

TABLE B

| | |
|---|---|
| B1 | 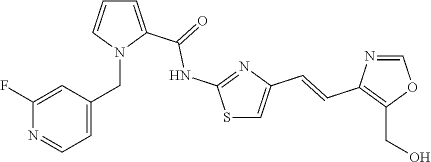 [SMILES: O=C(Nc1nc(/C=C/c2ncoc2CO)cs1)c1cccn1Cc1ccnc(F)c1] |
| B2 | 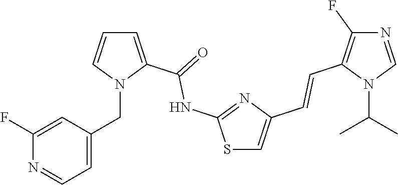 [SMILES: CC(C)n1cnc(F)c1/C=C/c1csc(NC(=O)c2cccn2Cc2ccnc(F)c2)n1] |
| B3 | 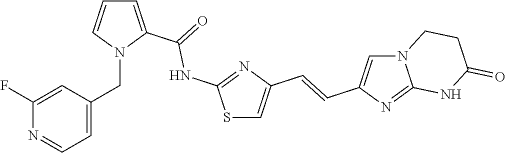 [SMILES: O=C1CCn2cc(/C=C/c3csc(NC(=O)c4cccn4Cc4ccnc(F)c4)n3)nc2N1] |
| B4 | 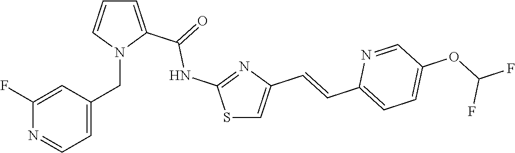 [SMILES: O=C(Nc1nc(/C=C/c2ccc(OC(F)F)cn2)cs1)c1cccn1Cc1ccnc(F)c1] |
| B5 | 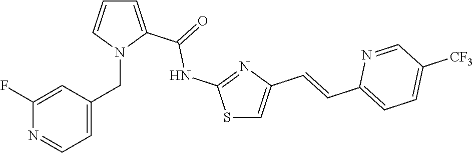 [SMILES: O=C(Nc1nc(/C=C/c2ccc(C(F)(F)F)cn2)cs1)c1cccn1Cc1ccnc(F)c1] |
| B6 | 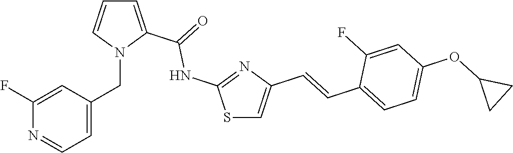 [SMILES: O=C(Nc1nc(/C=C/c2ccc(OC3CC3)cc2F)cs1)c1cccn1Cc1ccnc(F)c1] |
| B7 | 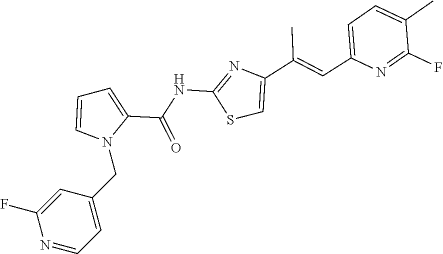 [SMILES: C/C(=C\c1ccc(C)c(F)n1)c1csc(NC(=O)c2cccn2Cc2ccnc(F)c2)n1] |
| B8 | 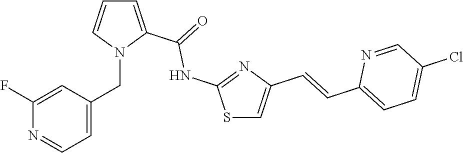 [SMILES: O=C(Nc1nc(/C=C/c2ccc(Cl)cn2)cs1)c1cccn1Cc1ccnc(F)c1] |
| B9 | 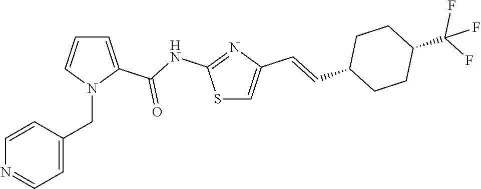 [SMILES: O=C(Nc1nc(/C=C/[C@H]2CC[C@@H](C(F)(F)F)CC2)cs1)c1cccn1Cc1ccncc1] |
| B10 | 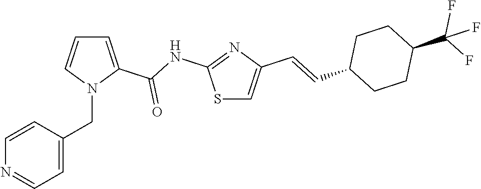 [SMILES: O=C(Nc1nc(/C=C/[C@H]2CC[C@H](C(F)(F)F)CC2)cs1)c1cccn1Cc1ccncc1] |
| B11 | 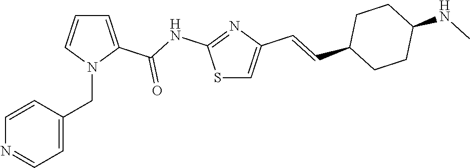 [SMILES: CN[C@H]1CC[C@@H](/C=C/c2csc(NC(=O)c3cccn3Cc3ccncc3)n2)CC1] |
| B12 | 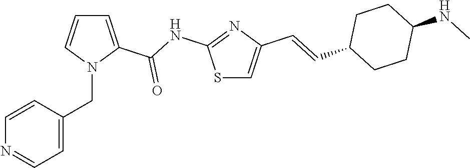 [SMILES: CN[C@H]1CC[C@H](/C=C/c2csc(NC(=O)c3cccn3Cc3ccncc3)n2)CC1] |
| B13 | 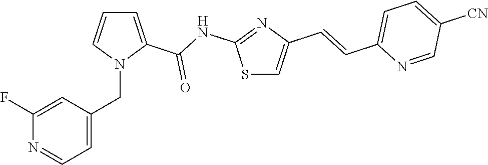 [SMILES: N#Cc1ccc(/C=C/c2csc(NC(=O)c3cccn3Cc3ccnc(F)c3)n2)nc1] |
| B14 | 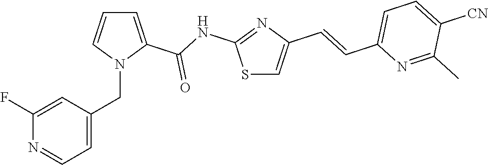 [SMILES: Cc1nc(/C=C/c2csc(NC(=O)c3cccn3Cc3ccnc(F)c3)n2)ccc1C#N] |
| B15 | 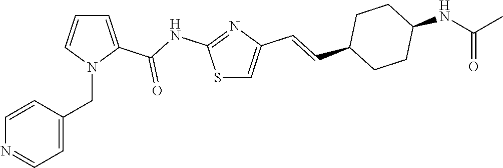 [SMILES: CC(=O)N[C@H]1CC[C@@H](/C=C/c2csc(NC(=O)c3cccn3Cc3ccncc3)n2)CC1] |
| B16 | 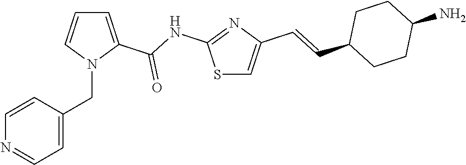 [SMILES: N[C@H]1CC[C@@H](/C=C/c2csc(NC(=O)c3cccn3Cc3ccncc3)n2)CC1] |
| B17 | 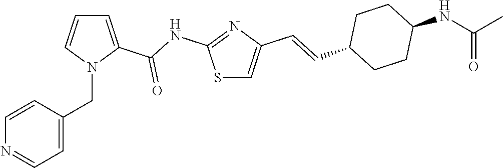 [SMILES: CC(=O)N[C@H]1CC[C@H](/C=C/c2csc(NC(=O)c3cccn3Cc3ccncc3)n2)CC1] |
| B18 | 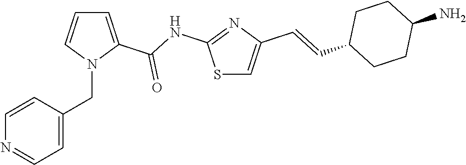 [SMILES: N[C@H]1CC[C@H](/C=C/c2csc(NC(=O)c3cccn3Cc3ccncc3)n2)CC1] |
| B19 | 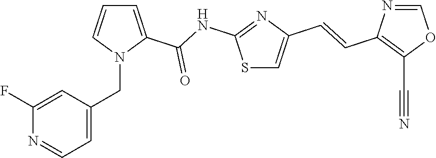 [SMILES: N#Cc1ocnc1/C=C/c1csc(NC(=O)c2cccn2Cc2ccnc(F)c2)n1] |
| B20 | 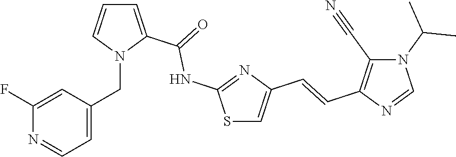 [SMILES: CC(C)n1cnc(/C=C/c2csc(NC(=O)c3cccn3Cc3ccnc(F)c3)n2)c1C#N] |
| B21 | 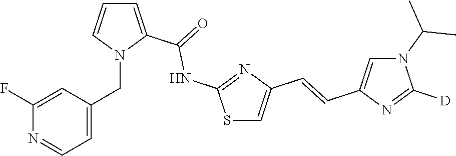 [SMILES: [2H]c1nc(/C=C/c2csc(NC(=O)c3cccn3Cc3ccnc(F)c3)n2)cn1C(C)C] |
| B22 | 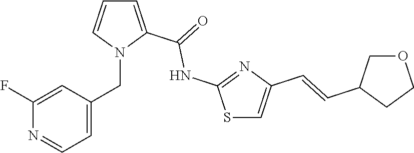 [SMILES: O=C(Nc1nc(/C=C/C2CCOC2)cs1)c1cccn1Cc1ccnc(F)c1] |
| B23 | 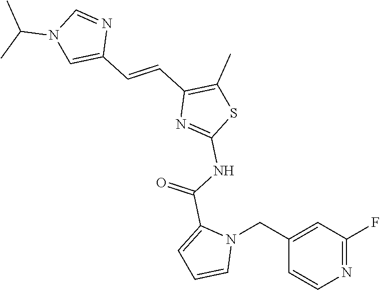 [SMILES: Cc1sc(NC(=O)c2cccn2Cc2ccnc(F)c2)nc1/C=C/c1cn(C(C)C)cn1] |
| B24 | 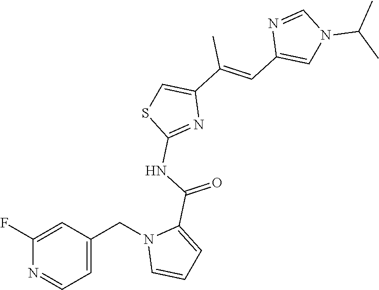 [SMILES: C/C(=C\c1cn(C(C)C)cn1)c1csc(NC(=O)c2cccn2Cc2ccnc(F)c2)n1] |
| B25 | 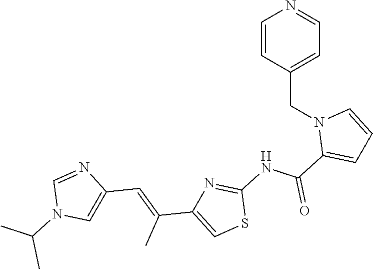 [SMILES: C/C(=C\c1cn(C(C)C)cn1)c1csc(NC(=O)c2cccn2Cc2ccncc2)n1] |
| B26 | 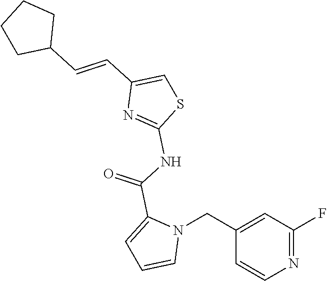 [SMILES: O=C(Nc1nc(/C=C/C2CCCC2)cs1)c1cccn1Cc1ccnc(F)c1] |
| B27 | 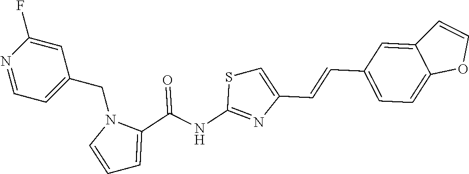 [SMILES: O=C(Nc1nc(/C=C/c2ccc3occc3c2)cs1)c1cccn1Cc1ccnc(F)c1] |
| B28 | 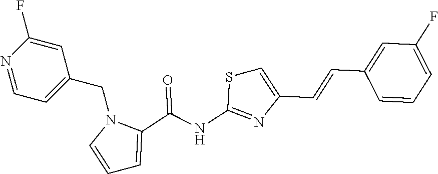 [SMILES: O=C(Nc1nc(/C=C/c2cccc(F)c2)cs1)c1cccn1Cc1ccnc(F)c1] |
| B29 | 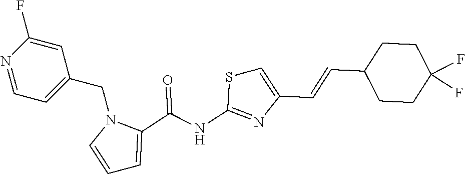 [SMILES: O=C(Nc1nc(/C=C/C2CCC(F)(F)CC2)cs1)c1cccn1Cc1ccnc(F)c1] |

Compounds as used herein, reference to an element, whether by description or chemical structure, encompasses all isotopes of that element unless otherwise described. By way of example, the term “hydrogen” or “H” in a chemical structure as used herein is understood to encompass, for example, not only ¹H, but also deuterium (²H or D), tritium (³H), and mixtures thereof unless otherwise denoted by use of a specific isotope. Other specific non-limiting examples of elements for which isotopes are encompassed include carbon, phosphorous, iodine, and fluorine.

The compounds disclosed herein can be present as a pharmaceutically acceptable salt. The term “pharmaceutically acceptable salt” refers to the relatively non-toxic, inorganic and organic acid addition salts of a compound provided herein. These salts can be prepared in situ during the final isolation and purification of a compound provided herein, or by separately reacting the compound in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, laurylsulphonate salts, and amino acid salts, and the like. (See, for example, Berge et al. (1977) “Pharmaceutical Salts”, J. Pharm. Sci. 66: 1-19.)

In some embodiments, a compound provided herein may contain one or more acidic functional groups and, thus, is capable of forming pharmaceutically acceptable salts with pharmaceutically acceptable bases. The term “pharmaceutically acceptable salts” in these instances refers to the relatively non-toxic inorganic and organic base addition salts of a compound provided herein. These salts can likewise be prepared in situ during the final isolation and purification of the compound, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary, or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like (see, for example, Berge et al., supra).

Without being bound by any particular theory, the compounds described herein inhibit protein secretion by binding to and disabling components of the translocon, including but not limited to Sec61, and in some cases, disrupting in a sequence specific fashion interactions between the nascent signaling sequence of translated proteins with components of the translocon including but not limited to Sec61.

The compounds described herein can advantageously inhibit the secretion of a protein of interest with an IC50 of up to 5 μM, or up to 3 μM, or up to 1 μM. In various cases, the compounds disclosed herein can inhibit the secretion of TNFα with an IC50 of up to 5 μM, or up to 3 μM, or up to 1 μM. In various cases, the compounds disclosed herein can inhibit the secretion of Her3 with an IC50 of up to 5 μM, or up to 3 μM, or up to 1 μM. In some cases, the compounds disclosed herein can inhibit the secretion of IL2 with an IC50 of up to 5 μM, or up to 3 μM, or up to 1 μM. In various cases, the compounds disclosed herein can inhibit the secretion of PD-1 with an IC50 of up to 5 μM, or up to 3 μM, or up to 1 μM.

In some cases, the compounds of Formula (I) are selective for PD1, i.e., they inhibit secretion of PD1 more than they inhibit secretion of another protein—such as, for example, IL2. Selectivity for a protein of interest can be assessed in line with the examples below. For example, a compound's IC₅₀ can be measured for PD1 and a second protein (e.g., IL2), and the selectivity of that compound can be calculated as a ratio of the relevant IC₅₀s. In some cases, the selectivity of the compound of Formula (I) for PD1, compared to IL2, is at least 20:1, or can be at least 30:1, 40:1, 50:1, or 60:1. In some cases, the selectivity for PD1 over IL2 is at least 100:1.

In various cases, the compounds of Formula (I) have a liver microsome stability of at least 25% after exposure of 30 minutes. Liver microsome stability can be assessed in line with the discussion in the examples below. The liver microsomes can be from e.g., mouse, rat, monkey and/or human. In some cases, the liver microsomes are human liver microsomes. Stability indicates the amount of the compound still present after a certain exposure time to the liver microsome.

In various cases, the compounds disclosed herein have an oral bioavailability of at least 10% (i.e., F≥10%), and in some cases is at least 20%, 25%, 30%, 40%, or 50%. Oral bioavailability can be assessed in line with the examples below, where a subject is administered a compound via iv administration and via oral administration, and the serum of the subject is analyzed, with AUC of the compound's concentration indicating the availability of the compound, and the iv administration indicating a 100% availability.

### Chemical Definitions

The compounds disclosed herein include all pharmaceutically acceptable isotopically-labeled compounds wherein one or more atoms of the compounds disclosed herein are replaced by atoms having the same atomic number, but an atomic mass or mass number different from the atomic mass or mass number usually found in nature, examples of which include isotopes of hydrogen, such as ²H and ³H. In some cases, one or more hydrogen atoms of the compounds disclosed herein are specifically deuterium (²H).

As used herein, the term “alkyl” refers to straight chained and branched saturated hydrocarbon groups containing one to thirty carbon atoms, for example, one to twenty carbon atoms, or one to ten carbon atoms. The term Cₙ means the alkyl group has “n” carbon atoms. For example, C₄alkyl refers to an alkyl group that has 4 carbon atoms. C₁₋₆alkyl refers to an alkyl group having a number of carbon atoms encompassing the entire range (i.e., 1 to 6 carbon atoms), as well as all subgroups (e.g., 1-5, 2-5, 1-4, 2-5, 1, 2, 3, 4, 5, and 6 carbon atoms). Nonlimiting examples of alkyl groups include, methyl, ethyl, n-propyl, isopropyl, n-butyl, sec-butyl (2-methylpropyl), and t-butyl (1, 1-dimethylethyl). Unless otherwise indicated, an alkyl group can be an unsubstituted alkyl group or a substituted alkyl group.

As used herein, the term “alkylene” refers to a bivalent saturated aliphatic radical. The term Cₙ means the alkylene group has “n” carbon atoms. For example, C₁₋₆alkylene refers to an alkylene group having a number of carbon atoms encompassing the entire range, as well as all subgroups, as previously described for “alkyl” groups.

As used herein, the term “carbocycle” refers to an aromatic or nonaromatic (i.e., fully or partially saturated) ring in which each atom of the ring is carbon. A carbocycle can include, for example, from three to ten carbon atoms, four to eight carbon atoms, or five to six carbon atoms. As used herein, the term “carbocycle” also includes polycyclic ring systems having two or more cyclic rings in which two or more carbons are common to two adjoining rings wherein at least one of the rings is carbocyclic, e.g., the other cyclic rings can be cycloalkyls, cycloalkenyls, aryls, heteroaryls, and/or heterocycles. As used herein, the term “heterocycle” is defined similarly as carbocycle, except the ring contains one to four heteroatoms independently selected from oxygen, nitrogen, and sulfur. For example, a heterocycle can be a 3-10 membered aromatic or non-aromatic ring having 1 or 2 heteroatoms selected from N, O, and S. As another example, a heterocycle can be a 5-6 membered ring having 1 or 2 ring heteroatoms selected from N, O, and S. Nonlimiting examples of heterocycle groups include piperdine, tetrahydrofuran, tetrahydropyran, dihydrofuran, morpholine, oxazepaneyl, thiazole, pyrrole, and pyridine.

As used herein, the term “cycloalkyl” specifically refers to a non-aromatic carbocycle. The term Cₙ means the cycloalkyl group has “n” carbon atoms. For example, C₅ cycloalkyl refers to a cycloalkyl group that has 5 carbon atoms in the ring. C₅₋₈ cycloalkyl refers to cycloalkyl groups having a number of carbon atoms encompassing the entire range (i.e., 5 to 10 carbon atoms), as well as all subgroups (e.g., 5-10, 5-9, 5-8, 5-6, 6-8, 7-8, 5-7, 5, 6, 7, 8, 9 and 10 carbon atoms). Nonlimiting examples of cycloalkyl groups include cyclopropyl, cyclobutyl, cyclopentyl, cyclohexyl, cycloheptyl, and cyclooctyl. Unless otherwise indicated, a cycloalkyl group can be an unsubstituted cycloalkyl group or a substituted cycloalkyl group.

Carbocyclic and heterocyclic groups can be saturated or partially unsaturated ring systems optionally substituted with, for example, one to three groups, independently selected alkyl, alkoxy, alkyleneOH, C(O)NH₂, NH₂, oxo (═O), aryl, haloalkyl, haloalkoxy, C(O)-alkyl, SO₂alkyl, halo, OH, NHC₁₋₃alkylene-aryl, OC₁₋₃alkylene-aryl, C₁₋₃alkylene-aryl, and C₃₋₆heterocycloalkyl having 1-3 heteroatoms selected from N, O, and S. Heterocyclic groups optionally can be further N-substituted as described herein. Other substituents contemplated for the disclosed rings is provided elsewhere in this disclosure.

As used herein, the term “hydroxy” or “hydroxyl” as used herein refers to an “—OH” group. Accordingly, a “hydroxyalkyl” refers to an alkyl group substituted with one or more —OH groups.

As used herein, the term “alkoxy” or “alkoxyl” refers to a “—O-alkyl” group.

As used herein, the term “halo” is defined as fluoro, chloro, bromo, and iodo. Accordingly, a “haloalkyl” refers to an alkyl group substituted with one or more halo atoms. A “haloalkoxy” refers to an alkoxy group that is substituted with one or more halo atoms.

The chemical structures having one or more stereocenters depicted with dashed and bold wedged bonds (i.e., [symbol] and [symbol] ) are meant to indicate absolute stereochemistry of the stereocenter(s) present in the chemical structure. Bonds symbolized by a simple line do not indicate a stereo-preference. Bonds symbolized by dashed or bold straight bonds (i.e., [symbol] and [symbol] ) are meant to indicate a relative stereochemistry of the stereocenter(s) present in the chemical structure. Unless otherwise indicated to the contrary, chemical structures that include one or more stereocenters which are illustrated herein without indicating absolute or relative stereochemistry, encompass all possible stereoisomeric forms of the compound (e.g., diastereomers, enantiomers) and mixtures thereof. Structures with a single bold or dashed wedged line, and at least one additional simple line, encompass a single enantiomeric series of all possible diastereomers. Similarly, the chemical structures having alkenyl groups are meant to encompass both cis and trans orientations, or when substituted, E- and Z-isomers of the chemical structure.

### Synthesis of Protein Secretion Inhibitors

The compounds provided herein can be synthesized using conventional techniques readily available starting materials known to those skilled in the art. In general, the compounds provided herein are conveniently obtained via standard organic chemistry synthesis methods.

Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

In general, the compounds of the disclosure can be synthesized in line with the examples shown below. For example, the compounds can be prepared by alkylation of the appropriate amine having a carboxyl group, with appropriate protecting groups as necessary. The intermediate can be saponified, for example, to expose a reactive carboxylate. Then, amide coupling between the appropriate amine and the free carboxylate can occur.

The amine for the amide coupling noted above can be prepared via known synthetic techniques using appropriate starting materials and protecting groups, as necessary.

Further modifications can be performed, e.g., to introduce additional substituents such as halo groups or alkyl groups.

### Methods of Use

The compounds disclosed herein can inhibit protein secretion of a protein of interest. The compounds disclosed herein can interfere with the Sec61 protein secretion machinery of a cell. In some cases, a compound as disclosed herein inhibits secretion of one or more of TNFα, IL2, Her3, and PD-1, or each of TNFα, IL2, Her3, and PD-1. In some cases, the compounds disclosed herein inhibit secretion of PD-1. Protein secretion activity can be assessed in a manner as described in the Examples section below.

As used herein, the term “inhibitor” is meant to describe a compound that blocks or reduces an activity of a pharmacological target (for example, a compound that inhibits Sec61 function in the protein secretion pathway). An inhibitor can act with competitive, uncompetitive, or noncompetitive inhibition. An inhibitor can bind reversibly or irreversibly, and therefore, the term includes compounds that are suitable substrates of a protein or enzyme. An inhibitor can modify one or more sites on or near the active site of the protein, or it can cause a conformational change elsewhere on the enzyme. The term inhibitor is used more broadly herein than scientific literature so as to also encompass other classes of pharmacologically or therapeutically useful agents, such as agonists, antagonists, stimulants, co-factors, and the like.

Thus, provided herein are methods of inhibiting protein secretion in a cell. In these methods, a cell is contacted with a compound described herein, or pharmaceutical composition thereof, in an amount effective to inhibit secretion of the protein of interest. In some embodiments, the cell is contacted in vitro. In various embodiments, the cell is contacted in vivo. In various embodiments, the contacting includes administering the compound or pharmaceutical composition to a subject.

The biological consequences of Sec61 inhibition are numerous. For example, Sec61 inhibition has been suggested for the treatment or prevention of inflammation and/or cancer in a subject. Therefore, pharmaceutical compositions for Sec61 specific compounds, provide a means of administering a drug to a subject and treating these conditions. As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound of the disclosure to an individual in need of such treatment. Within the meaning of the disclosure, “treatment” also includes relapse prophylaxis or phase prophylaxis, as well as the treatment of acute or chronic signs, symptoms and/or malfunctions. The treatment can be orientated symptomatically, for example, to suppress symptoms. It can be effected over a short period, be oriented over a medium term, or can be a long-term treatment, for example within the context of a maintenance therapy. As used herein, the terms “prevent,” “preventing,” “prevention,” are art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. As used herein, the terms “patient” and “subject” may be used interchangeably and mean animals, such as dogs, cats, cows, horses, and sheep (i.e., non-human animals) and humans. Particular patients are mammals (e.g., humans). The term patient includes males and females.

Inhibition of Sec61-mediated secretion of inflammatory proteins (e.g., TNFα) can disrupt inflammation signaling. Thus, provided herein is a method of treating inflammation in a subject by administering to the subject a therapeutically effective amount of a compound described herein.

Further, the viability of cancer cells relies upon increased protein secretion into the ER for survival. Therefore, non-selective or partially selective inhibition of Sec61 mediated protein secretion may inhibit tumor growth. Alternatively, in the immune-oncology setting, selective secretion inhibitors of known secreted immune checkpoints proteins (e.g., PD-1, TIM-3, LAG3, etc.) can result in activation of the immune system to against various cancers.

Accordingly, also provided herein are methods of treating cancer in a subject by administering to the subject a therapeutically effective amount of a compound described herein or a pharmaceutically acceptable salt thereof. Specifically contemplated cancers that can be treated using the compounds and compositions described herein include, but are not limited to melanoma, multiple myeloma, prostate, lung, non small cell lung carconimoa (NSCLC), squamous cell carcinoma, leukemia, acute myelogenous leukemia, chronic myelogenous leukemia, lymphoma, NPM/ALK-transformed anaplastic large cell lymphoma, renal cell carcinoma, rhabdomyosarcoma, ovarian cancer, endometrial cancer, small cell carcinoma, adenocarcinoma, gastric carcinoma, hepatocellular carcinoma, pancreatic cancer, thyroid carcinoma, anaplastic large cell lymphoma, hemangioma, head and neck cancer, bladder, and colorectal cancers.

The compounds described herein are also contemplated to be used in the prevention and/or treatment of a multitude of diseases including, but not limited to, proliferative diseases, neurotoxic/degenerative diseases, ischemic conditions, autoimmune and autoinflammatory disorders, inflammation, immune-related diseases, HIV, cancers, organ graft rejection, septic shock, viral and parasitic infections, conditions associated with acidosis, macular degeneration, pulmonary conditions, muscle wasting diseases, fibrotic diseases, bone and hair growth diseases.

Examples of proliferative diseases or conditions include diabetic retinopathy, macular degeneration, diabetic nephropathy, glomerulosclerosis, IgA nephropathy, cirrhosis, biliary atresia, congestive heart failure, scleroderma, radiation-induced fibrosis, and lung fibrosis (idiopathic pulmonary fibrosis, collagen vascular disease, sarcoidosis, interstitial lung diseases and extrinsic lung disorders).

Inflammatory diseases include acute (e.g., bronchitis, conjunctivitis, myocarditis, pancreatitis) and chronic conditions (e.g., chronic cholecstitis, bronchiectasis, aortic valve stenosis, restenosis, psoriasis and arthritis), along with conditions associated with inflammation such as fibrosis, infection and ischemia.

Immunodeficiency disorders occur when a part of the immune system is not working properly or is not present. They can affect B lymophyctes, T lymphocytes, or phagocytes and be either inherited (e.g., IgA deficiency, severe combined immunodeficiency (SCID), thymic dysplasia and chronic granulomatous) or acquired (e.g., acquired immunodeficiency syndrome (AIDS), human immunodeficiency virus (HIV) and drug-induced immunodeficiencies). Immune-related conditions include allergic disorders such as allergies, asthma and atopic dermatitis like eczema. Other examples of such immune-related conditions include lupus, rheumatoid arthritis, scleroderma, ankylosing spondylitis, dermatomyositis, psoriasis, multiple sclerosis and inflammatory bowel disease (such as ulcerative colitis and Crohn's disease).

Tissue/organ graft rejection occurs when the immune system mistakenly attacks the cells being introduced to the host's body. Graft versus host disease (GVHD), resulting from allogenic transplantation, arises when the T cells from the donor tissue go on the offensive and attack the host's tissues. In all three circumstances, autoimmune disease, transplant rejection and GVHD, modulating the immune system by treating the subject with a compound or composition of the disclosure could be beneficial.

Also provided herein are methods of treating an autoimmune disease in a patient comprising administering a therapeutically effective amount of the compound described herein. An “autoimmune disease” as used herein is a disease or disorder arising from and directed against an individual's own tissues. Examples of autoimmune diseases include, but are not limited to, inflammatory responses such as inflammatory skin diseases including psoriasis and dermatitis (e.g., atopic dermatitis); systemic scleroderma and sclerosis; responses associated with inflammatory bowel disease (such as Crohn's disease and ulcerative colitis); respiratory distress syndrome (including adult respiratory distress syndrome (ARDS)); dermatitis; meningitis; encephalitis; uveitis; colitis; glomerulonephritis; allergic conditions such as eczema and asthma and other conditions involving infiltration of T cells and chronic inflammatory responses; atherosclerosis; leukocyte adhesion deficiency; rheumatoid arthritis; systemic lupus erythematosus (SLE); diabetes mellitus (e.g., Type I diabetes mellitus or insulin dependent diabetes mellitus); multiple sclerosis; Reynaud's syndrome; autoimmune thyroiditis; allergic encephalomyelitis; Sjorgen's syndrome; juvenile onset diabetes; and immune responses associated with acute and delayed hypersensitivity mediated by cytokines and T-lymphocytes typically found in tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia (including, but not limited to cryoglobinemia or Coombs positive anemia); myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia. Compounds provided herein may be useful for the treatment of conditions associated with inflammation, including, but not limited to COPD, psoriasis, asthma, bronchitis, emphysema, and cystic fibrosis.

Also provided herein is the use of a compound as disclosed herein for the treatment of neurodegenerative diseases. Neurodegenerative diseases and conditions includes, but not limited to, stroke, ischemic damage to the nervous system, neural trauma (e.g., percussive brain damage, spinal cord injury, and traumatic damage to the nervous system), multiple sclerosis and other immune-mediated neuropathies (e.g., Guillain-Barre syndrome and its variants, acute motor axonal neuropathy, acute inflammatory demyelinating polyneuropathy, and Fisher Syndrome), HIV/AIDS dementia complex, axonomy, diabetic neuropathy, Parkinson's disease, Huntington's disease, multiple sclerosis, bacterial, parasitic, fungal, and viral meningitis, encephalitis, vascular dementia, multi-infarct dementia, Lewy body dementia, frontal lobe dementia such as Pick's disease, subcortical dementias (such as Huntington or progressive supranuclear palsy), focal cortical atrophy syndromes (such as primary aphasia), metabolic-toxic dementias (such as chronic hypothyroidism or B12 deficiency), and dementias caused by infections (such as syphilis or chronic meningitis).

Further guidance for using compounds and compositions described for inhibiting protein secretion can be found in the Examples section, below.

### Pharmaceutical Compositions and Administration

Provided herein is disclosure for the manufacture and use of pharmaceutical compositions, which include one or more of the compounds as disclosed herein. Also included are the pharmaceutical compositions themselves. Pharmaceutical compositions typically include a pharmaceutically acceptable carrier. Thus, provided herein are pharmaceutical compositions that include a compound described herein and one or more pharmaceutically acceptable carriers.

The phrase “pharmaceutically acceptable” is employed herein to refer to those ligands, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material. As used herein the language “pharmaceutically acceptable carrier” includes buffer, sterile water for injection, solvents, dispersion media, coatings, antibacterial and antifungal agents, isotonic and absorption delaying agents, and the like, compatible with pharmaceutical administration. Each carrier must be “acceptable” in the sense of being compatible with the other ingredients of the composition and not injurious to the patient. Some examples of materials which can serve as pharmaceutically acceptable carriers include: (1) sugars, such as lactose, glucose, and sucrose; (2) starches, such as corn starch, potato starch, and substituted or unsubstituted β-cyclodextrin; (3) cellulose, and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical compositions. In certain embodiments, pharmaceutical compositions provided herein are non-pyrogenic, i.e., do not induce significant temperature elevations when administered to a patient.

Wetting agents, emulsifiers, and lubricants, such as sodium lauryl sulfate and magnesium stearate, as well as coloring agents, release agents, coating agents, sweetening, flavoring, and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

A pharmaceutical composition may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include tonicity-adjusting agents, such as sugars and the like into the compositions. In addition, prolonged absorption of the injectable pharmaceutical form may be brought about by the inclusion of agents which delay absorption such as aluminum monostearate and gelatin.

In some cases, in order to prolong the effect of one or more compounds provided herein, it is desirable to slow the absorption of the compound from subcutaneous or intramuscular injection. For example, delayed absorption of a parenterally administered compound can be accomplished by dissolving or suspending the compound in an oil vehicle.

Compositions prepared as described herein can be administered in various forms, depending on the disorder to be treated and the age, condition, and body weight of the patient, as is well known in the art. For example, where the compositions are to be administered orally, they may be formulated as tablets, capsules, granules, powders, or syrups; or for parenteral administration, they may be formulated as injections (intravenous, intramuscular, or subcutaneous), drop infusion preparations, or suppositories. For application by the ophthalmic mucous membrane route, they may be formulated as eye drops or eye ointments. These compositions can be prepared by conventional means in conjunction with the methods described herein, and, if desired, the active ingredient may be mixed with any conventional additive or excipient, such as a binder, a disintegrating agent, a lubricant, a corrigent, a solubilizing agent, a suspension aid, an emulsifying agent, or a coating agent.

Compositions suitable for oral administration may be in the form of capsules (e.g., gelatin capsules), cachets, pills, tablets, lozenges (using a flavored basis, usually sucrose and acacia or tragacanth), powders, troches, granules, or as a solution or a suspension in an aqueous or non-aqueous liquid, or as an oil-in-water or water-in-oil liquid emulsion, or as an elixir or syrup, or as pastilles (using an inert matrix, such as gelatin and glycerin, or sucrose and acacia) and/or as mouthwashes, and the like, each containing a predetermined amount of a compound provided herein as an active ingredient. A composition may also be administered as a bolus, electuary, or paste. Oral compositions generally include an inert diluent or an edible carrier.

Pharmaceutically compatible binding agents, and/or adjuvant materials can be included as part of an oral composition. In solid dosage forms for oral administration (capsules, tablets, pills, dragees, powders, granules, and the like), the active ingredient can be mixed with one or more pharmaceutically acceptable carriers, such as sodium citrate or dicalcium phosphate, and/or any of the following: (1) fillers or extenders, such as starches, cyclodextrins, lactose, sucrose, saccharin, glucose, mannitol, and/or silicic acid; (2) binders, such as, for example, carboxymethylcellulose, microcrystalline cellulose, gum tragacanth, alginates, gelatin, polyvinyl pyrrolidone, sucrose, and/or acacia; (3) humectants, such as glycerol; (4) disintegrating agents, such as agar-agar, calcium carbonate, potato, corn, or tapioca starch, alginic acid, Primogel, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, Sterotes, solid polyethylene glycols, sodium lauryl sulfate, and mixtures thereof; (10) a glidant, such as colloidal silicon dioxide; (11) coloring agents; and (12) a flavoring agent such as peppermint, methyl salicylate, or orange flavoring. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols, and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of a powdered compound moistened with an inert liquid diluent.

Tablets, and other solid dosage forms, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, microspheres, and/or nanoparticles. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents, and emulsifiers such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols, and fatty acid esters of sorbitan, and mixtures thereof.

Besides inert diluents, the oral compositions can also include adjuvants such as wetting agents, emulsifying and suspending agents, sweetening, flavoring, coloring, perfuming, and preservative agents.

Suspensions, in addition to the active compound(s) may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions suitable for parenteral administration can include one or more compounds provided herein in combination with one or more pharmaceutically acceptable sterile aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the composition isotonic with the blood of the intended recipient or suspending or thickening agents.

Examples of suitable aqueous and nonaqueous carriers which may be employed in the pharmaceutical compositions provided herein include water for injection (e.g., sterile water for injection), bacteriostatic water, ethanol, polyols (such as glycerol, propylene glycol, polyethylene glycol such as liquid polyethylene glycol, and the like), sterile buffer (such as citrate buffer), and suitable mixtures thereof, vegetable oils, such as olive oil, injectable organic esters, such as ethyl oleate, and Cremophor EL™ (BASF, Parsippany, NJ). In all cases, the composition must be sterile and should be fluid to the extent that easy syringability exists. Proper fluidity can be maintained, for example, by the use of coating materials, such as lecithin, by the maintenance of the required particle size in the case of dispersions, and by the use of surfactants.

The composition should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the methods of preparation are freeze-drying (lyophilization), which yields a powder of the active ingredient plus any additional desired ingredient from a previously sterile-filtered solution thereof.

Injectable depot forms can be made by forming microencapsule or nanoencapsule matrices of a compound provided herein in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable compositions are also prepared by entrapping the drug in liposomes, microemulsions or nanoemulsions, which are compatible with body tissue.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798. Additionally, intranasal delivery can be accomplished, as described in, inter alia, Hamajima et al., Clin. Immunol. Immunopathol., 88(2), 205-10 (1998). Liposomes (e.g., as described in U.S. Pat. No. 6,472,375, which is incorporated herein by reference in its entirety), microencapsulation and nanoencapsulation can also be used. Biodegradable targetable microparticle delivery systems or biodegradable targetable nanoparticle delivery systems can also be used (e.g., as described in U.S. Pat. No. 6,471,996, which is incorporated herein by reference in its entirety).

Systemic administration of a therapeutic compound as described herein can also be by transmucosal or transdermal means. Dosage forms for the topical or transdermal administration of a compound provided herein include powders, sprays, ointments, pastes, creams, lotions, gels, solutions, patches, and inhalants. The active component may be mixed under sterile conditions with a pharmaceutically acceptable carrier, and with any preservatives, buffers, or propellants which may be required. For transmucosal or transdermal administration, penetrants appropriate to the barrier to be permeated are used in the composition. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The ointments, pastes, creams, and gels may contain, in addition to one or more compounds provided herein, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound provided herein, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

A compound provided herein can be administered by aerosol. This is accomplished by preparing an aqueous aerosol, liposomal preparation, or solid particles containing a compound or composition provided herein. A nonaqueous (e.g., fluorocarbon propellant) suspension could be used. In some embodiments, sonic nebulizers are used because they minimize exposing the agent to shear, which can result in degradation of the compound.

Ordinarily, an aqueous aerosol can be made by formulating an aqueous solution or suspension of the agent together with conventional pharmaceutically acceptable carriers and stabilizers. The carriers and stabilizers vary with the requirements of the particular composition, but typically include nonionic surfactants (TWEEN® (polysorbates), PLURONIC® (poloxamers), sorbitan esters, lecithin, CREMOPHOR® (polyethoxylates)), pharmaceutically acceptable co-solvents such as polyethylene glycol, innocuous proteins like serum albumin, sorbitan esters, oleic acid, lecithin, amino acids such as glycine, buffers, salts, sugars, or sugar alcohols. Aerosols generally are prepared from isotonic solutions.

Transdermal patches have the added advantage of providing controlled delivery of a compound provided herein to the body. Such dosage forms can be made by dissolving or dispersing the agent in the proper medium. Absorption enhancers can also be used to increase the flux of the compound across the skin. The rate of such flux can be controlled by either providing a rate controlling membrane or dispersing the compound in a polymer matrix or gel.

The pharmaceutical compositions can also be prepared in the form of suppositories or retention enemas for rectal and/or vaginal delivery. Compositions presented as a suppository can be prepared by mixing one or more compounds provided herein with one or more suitable nonirritating excipients or carriers comprising, for example, cocoa butter, glycerides, polyethylene glycol, a suppository wax or a salicylate, which is solid at room temperature, but liquid at body temperature and, therefore, will melt in the rectum or vaginal cavity and release the active agent. Compositions which are suitable for vaginal administration also include pessaries, tampons, creams, gels, pastes, foams, or spray compositions containing such carriers as are known in the art to be appropriate.

A compound as disclosed herein can be prepared with carriers that will protect the compound against rapid elimination from the body, such as a controlled release composition, including implants and microencapsulated delivery systems. Biodegradable, biocompatible polymers can be used, such as ethylene vinyl acetate, polyanhydrides, polyglycolic acid, collagen, polyorthoesters, and polylactic acid. Such compositions can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811, which is incorporated herein by reference in its entirety.

As described above, the preparations of one or more compounds provided herein may be given orally, parenterally, topically, or rectally. They are, of course, given by forms suitable for each administration route. For example, they are administered in tablets or capsule form, by injection, inhalation, eye lotion, ointment, suppository, infusion; topically by lotion or ointment; and rectally by suppositories. In some embodiments, administration is oral.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection, and infusion.

The phrases “systemic administration”, “administered systemically”, “peripheral administration”, and “administered peripherally” as used herein mean the administration of a ligand, drug, or other material via route other than directly into the central nervous system, such that it enters the patient's system and thus, is subject to metabolism and other like processes, for example, subcutaneous administration.

A compound provided herein may be administered to humans and other animals for therapy by any suitable route of administration, including orally, nasally, as by, for example, a spray, rectally, intravaginally, parenterally, intracistemally, and topically, as by powders, ointments or drops, including buccally and sublingually. Regardless of the route of administration selected, a compound provided herein, which may be used in a suitable hydrated form, and/or the pharmaceutical compositions provided herein, is formulated into a pharmaceutically acceptable dosage form by conventional methods known to those of skill in the art. In another embodiment, the pharmaceutical composition is an oral solution or a parenteral solution. Another embodiment is a freeze-dried preparation that can be reconstituted prior to administration. As a solid, this composition may also include tablets, capsules or powders.

Actual dosage levels of the active ingredients in the pharmaceutical compositions provided herein may be varied so as to obtain “therapeutically effective amount,” which is an amount of the active ingredient effective to achieve the desired therapeutic response for a particular patient, composition, and mode of administration, without being toxic to the patient.

The concentration of a compound provided herein in a pharmaceutically acceptable mixture will vary depending on several factors, including the dosage of the compound to be administered, the pharmacokinetic characteristics of the compound(s) employed, and the route of administration. In some embodiments, the compositions provided herein can be provided in an aqueous solution containing about 0.1-10% w/v of a compound disclosed herein, among other substances, for parenteral administration. Typical dose ranges can include from about 0.01 to about 50 mg/kg of body weight per day, given in 1-4 divided doses. Each divided dose may contain the same or different compounds. The dosage will be a therapeutically effective amount depending on several factors including the overall health of a patient, and the composition and route of administration of the selected compound(s).

Dosage forms or compositions containing a compound as described herein in the range of 0.005% to 100% with the balance made up from non-toxic carrier may be prepared. Methods for preparation of these compositions are known to those skilled in the art. The contemplated compositions may contain 0.001%-100% active ingredient, in one embodiment 0.1-95%, in another embodiment 75-85%. Although the dosage will vary depending on the symptoms, age and body weight of the patient, the nature and severity of the disorder to be treated or prevented, the route of administration and the form of the drug, in general, a daily dosage of from 0.01 to 2000 mg of the compound is recommended for an adult human patient, and this may be administered in a single dose or in divided doses. The amount of active ingredient which can be combined with a carrier material to produce a single dosage form will generally be that amount of the compound which produces a therapeutic effect.

The pharmaceutical composition may be administered at once, or may be divided into a number of smaller doses to be administered at intervals of time. It is also noted that the dose of the compound can be varied over time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular patient, specific dosage regimens should be adjusted over time according to the individual need and the professional judgment of the person administering or supervising the administration of the compositions, and that the concentration ranges set forth herein are exemplary only and are not intended to limit the scope or practice of the embodimented compositions.

The precise time of administration and/or amount of the composition that will yield the most effective results in terms of efficacy of treatment in a given patient will depend upon the activity, pharmacokinetics, and bioavailability of a particular compound, physiological condition of the patient (including age, sex, disease type and stage, general physical condition, responsiveness to a given dosage, and type of medication), route of administration, etc. However, the above guidelines can be used as the basis for fine-tuning the treatment, e.g., determining the optimum time and/or amount of administration, which will require no more than routine experimentation consisting of monitoring the patient and adjusting the dosage and/or timing.

The pharmaceutical compositions can be included in a container, pack, or dispenser together with instructions for administration.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

It is to be understood that while the disclosure is read in conjunction with the detailed description thereof, the foregoing description is intended to illustrate and not limit the scope of the disclosure, which is defined by the scope of the appended claims. Other aspects, advantages, and modifications are within the scope of the following claims.

### EXAMPLES

The following examples are provided for illustration and are not intended to limit the scope of the disclosure in any way.

As used throughout these examples, common organic abbreviations are defined as follows:

| Abbreviation | Chemical | Abbreviation | Chemical |
|---|---|---|---|
| Ac | Acetyl | EtOAc | Ethyl acetate |
| ACN | Acetonitrile | EtOH | Ethanol |
| BOC or Boc | tert-Butoxycarbonyl | HOBT | Hydroxybenzotriazole |
| (Boc)₂O | Di-tert-butyl dicarbonate | LDA | Lithium diisopropylamide |
| CDI | 1,1′-Carbonyldiimidazole | mCBPA | meta-Chloroperoxybenzoic acid |
| Cy | Cyclohexyl | MeOH | Methanol |
| DBU | 1,8-Diazabicyclo[5.4.0]undec-7- | Ms₂O | Mesyl anhydride |
| | ene | NaHMDS | Sodium Hexamethyldisilazide |
| dba (e.g., | Dibenzylideneacetone | NBS | N-bromosuccinimide |
| Pd₂(dba)₃) | | NMI | 1-methylimidazole |
| DCC | N,N′-Dicyclohexylcarbodiimide | NMM | N-Methylmorpholine |
| DCE | 1,2-Dichloroethane | OAc | Acetyloxy |
| DCM | Methylene chloride | Pd/C | Palladium on activated carbon |
| DIEA/DIPEA | Diisopropylethylamine | PMBCl | p-Methoxylbenzyl chloride |
| DMF | N,N′-Dimethylformamide | PPh₃ | Triphenylphopshine |
| DMSO | Dimethylsulfoxide | RP | Reverse Phase |
| dtpf/dtbpf (e.g., | 1,1′-bis(di-tert- | Sat. | Saturated |
| Pd(dtpf)Cl₂) | butylphosphino)ferrocene | TBAF | Tetrabutylammonium fluoride |
| TBDPSCl | tert-Butyl(chloro)diphenylsilane | TEA or NEt₃ | Triethylamine |
| TCFH | N,N,N′,N′- | TFA | Trifluoroacetic acid |
| | tetramethylchloroformamidinium | THF | Tetrahydrofuran |
| | hexafluorophosphate | TMSOTf | Trimethylsilyl Triflate |

### SYNTHETIC EXAMPLES

### Amine Synthesis:

### Route 1:

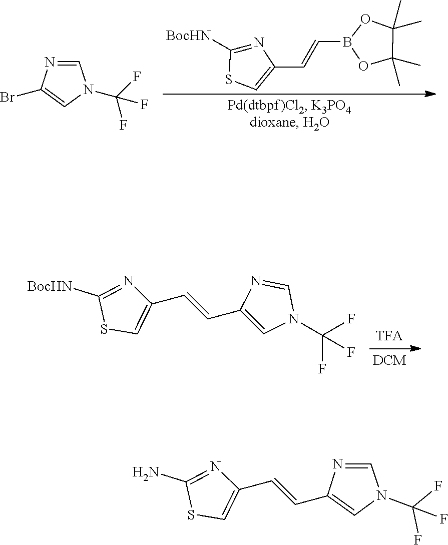 [SMILES: FC(F)(F)n1cnc(Br)c1 ; CC(C)(C)OC(=O)Nc1nc(/C=C/B2OC(C)(C)C(C)(C)O2)cs1 ; CC(C)(C)OC(=O)Nc1nc(/C=C/c2cn(C(F)(F)F)cn2)cs1 ; Nc1nc(/C=C/c2cn(C(F)(F)F)cn2)cs1]

### Step 1:

A vial with stir bar was charged with 4-bromo-1-(trifluoromethyl)imidazole (100 mg, 0.47 mmol, 1.00 equiv) and tert-butyl N-{4-[(E)-2-(4,4,5,5-tetramethyl-1,3,2-dioxaborolan-2-yl)ethenyl]-1,3-thiazol-2-yl}carbamate (196.63 mg, 0.56 mmol, 1.20 equiv), Pd(dtbpf)Cl₂ (60.64 mg, 0.09 mmol, 0.20 equiv) in dioxane (6 mL) and H₂O (2 mL) under nitrogen atmosphere. The vial was capped and placed in a 90° C. bath. The reaction mixture was stirred at 90° C. for 2 h. The resulting mixture was cooled to room temperature, poured into DCM (30 mL) and washed with brine (2×30 mL). The combined organic layers were dried over Na₂SO₄, filtered and concentrated in vacuo. The resulting crude material was purified via RP column to yield the desired product tert-butyl (E)-(4-(2-(1-(trifluoromethyl)-1H-imidazol-4-yl)vinyl)thiazol-2-yl)carbamate.

### Step 2:

A vial with stir bar was charged with tert-butyl N-{4-[(E)-2-[1-(trifluoromethyl)imidazol-4-yl]ethenyl]-1,3-thiazol-2-yl}carbamate (180 mg, 0.50 mmol, 1.00 equiv) in DCM (4 mL), TFA (2 mL) was added. The vial was capped and placed in a 25° C. bath. The reaction mixture was stirred at 25° C. for 1 h. The resulting mixture was concentrated under vacuum, and the pH value of the solution was adjusted to 8 with sat.NaHCO₃ (aq). The aqueous layer was extracted with DCM (4×20 mL) and washed with brine (1×20 mL). The combined organic layer was dried over Na₂SO₄, filtered and concentrated in vacuo. The resulting crude material was purified via silica gel column to yield the desired product (E)-4-(2-(1-(trifluoromethyl)-1H-imidazol-4-yl)vinyl)thiazol-2-amine.

The following intermediates for compounds listed in the table were prepared via a similar method:

| Compound | |
|---|---|
| # | Compound name |
| A4 | (E)-4-(2-(1-(1,1-difluoropropan-2-yl)-1H-imidazol-4- |
| | yl)vinyl)thiazol-2-amine |
| A5 | (E)-4-(2-(1-(difluoromethyl)-1H-imidazol-4- |
| | yl)vinyl)thiazol-2-amine |

### Route 2:

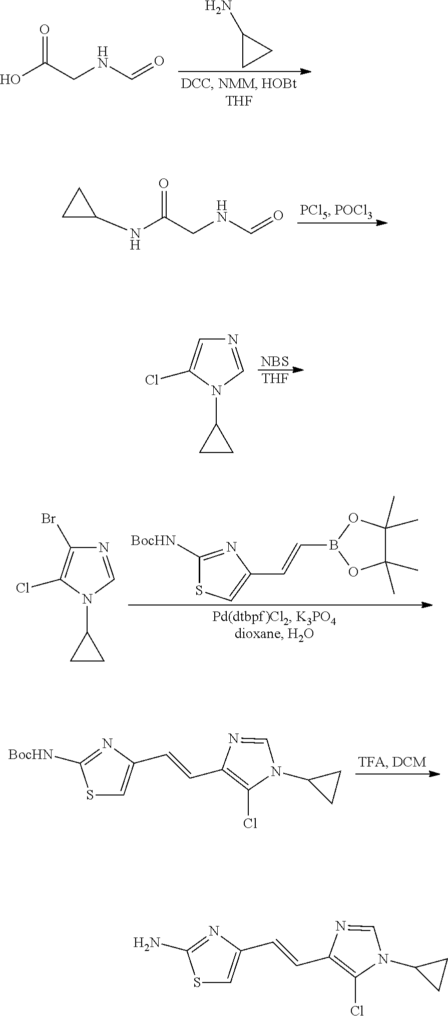

### Step 1:

A vial with stir bar was charged with formylglycine (5.00 g, 48.51 mmol, 1.00 equiv) and NMM (5.89 g, 58.21 mmol, 1.20 equiv), HOBT (7.21 g, 53.36 mmol, 1.10 equiv), DCC (11.01 g, 53.36 mmol, 1.10 equiv) in THE (200 mL, 0.24 M), and then cyclopropanamine (2.77 g, 48.51 mmol, 1.00 equiv) was added. The vial was capped and placed in a 25° C. bath. The reaction mixture was stirred at 25° C. for 12 h. The resulting mixture was filtered, the filter cake was washed with EtOAc (3×200 mL). The filtrate was concentrated under reduced pressure. The resulting crude material was purified via silica gel column to yield the desired product.

### Step 2:

A vial with stir bar was charged with N-cyclopropyl-2-formamidoacetamide (5 g, 35.17 mmol, 1.00 equiv) in POCl₃ (50 mL) at 0° C., PCl₅ (14.65 g, 70.34 mmol, 2.00 equiv) was added at 0° C. The vial was capped and placed in a 0° C. bath. The reaction mixture was stirred at 0° C. for 2 h. And then the resulting mixture was stirred for 2 h at 60° C. in a 60° C. bath. The reaction mixture was cooled to room temperature. The resulting mixture was concentrated under vacuum. The reaction was quenched by water. The resulting solution was extracted with (3×70 mL) of ethyl acetate, and the pH value of the aqueous layer was adjusted to 10 with NH₃·H₂O. The aqueous layer was extracted with EtOAc (3×100 mL) and washed with (2×100 mL) of brine. The organic layer was then dried over Na₂SO₄, filtered and concentrated in vacuo. The resulting crude material was used directly for next step without further purification.

### Step 3:

A vial with stir bar was charged with 5-chloro-1-cyclopropylimidazole (1.80 g, 12.62 mmol, 1.00 equiv) in THE (20 mL, 0.63 M), NBS (1.80 g, 10.10 mmol, 0.80 equiv) was added. The vial was capped and placed in a 25° C. bath. The reaction mixture was stirred at 25° C. for 1 h. The reaction was quenched by water. The aqueous layer was extracted with EtOAc (3×50 mL) and washed with (2×50 mL) of brine. The organic layer was then dried over Na₂SO₄, filtered and concentrated in vacuo. The resulting crude material was purified via RP column to yield the desired product.

### Step 4:

A vial with stir bar was charged with 4-bromo-5-chloro-1-cyclopropylimidazole (1.20 g, 5.42 mmol, 1.00 equiv) and tert-butyl N-{4-[(E)-2-(4,4,5,5-tetramethyl-1,3,2-dioxaborolan-2-yl)ethenyl]-1,3-thiazol-2-yl}carbamate (2.29 g, 6.50 mmol, 1.20 equiv), Pd(dtbpf)Cl₂ (699.94 mg, 1.08 mmol, 0.20 equiv), K₃PO₄ (3.45 g, 16.25 mmol, 3.00 equiv) in dioxane (30 mL) and H₂O (10 mL) under nitrogen atmosphere. The vial was capped and placed in a 90° C. bath. The reaction mixture was stirred at 90° C. for 2 h. The resulting mixture was cooled to room temperature, poured into DCM (150 mL) and washed with brine (2×80 mL). The combined organic layers were dried over Na₂SO₄, filtered and concentrated in vacuo. The resulting crude material was purified via silica gel column to yield the desired product.

### Step 5:

A vial with stir bar was charged with tert-butyl N-{4-[(E)-2-(5-chloro-1-cyclopropylimidazol-4-yl)ethenyl]-1,3-thiazol-2-yl}carbamate (550.00 mg, 1.50 mmol, 1.00 equiv) in DCM (4 mL), TFA (4 mL) was added. The vial was capped and placed in a 25° C. bath. The reaction mixture was stirred at 25° C. for 1 h. The resulting mixture was concentrated under vacuum, and the pH value of the solution was adjusted to 8 with sat.NaHCO₃ (aq). The aqueous layer was extracted with DCM (4×30 mL). The combined organic layer was dried over Na₂SO₄, filtered and concentrated in vacuo. The resulting crude material was purified via silica gel column to yield the desired product (E)-4-(2-(5-chloro-1-isopropyl-1H-imidazol-4-yl)vinyl)thiazol-2-amine.

### Route 3:

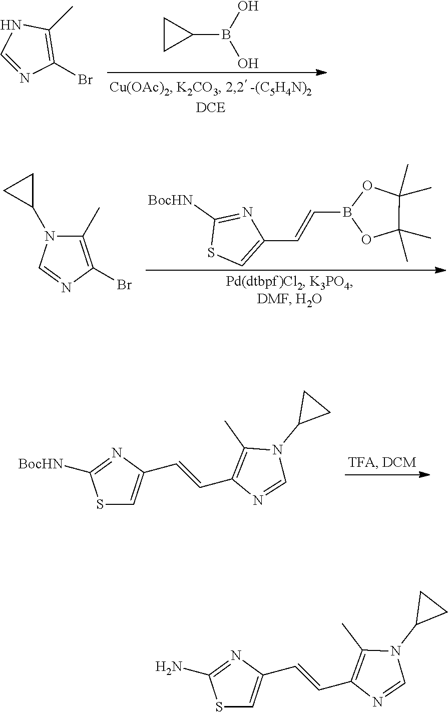

### Step 1:

A vial with stir bar was charged with 4-bromo-5-methyl-1H-imidazole (2.00 g, 12.42 mmol, 1.00 equiv), cyclopropylboronic acid (2.13 g, 24.84 mmol, 2.00 equiv), Cu(OAc)₂ (2.26 g, 12.42 mmol, 1.00 equiv), K₂CO₃ (3.46 g, 24.84 mmol, 2.00 equiv), bipyridyl (1.94 g, 12.42 mmol, 1.00 equiv) and DCE (50 mL, 0.25 M) under nitrogen atmosphere. The reaction flask was then vacuumed and flushed with oxygen, and the sequence was repeated twice. The vial was capped and placed in an 70° C. bath. The reaction mixture was stirred at 70° C. for 3 h under oxygen atmosphere using an oxygen balloon. The reaction mixture was cooled to room temperature. The reaction mixture was poured into DCM (150 mL) and washed with H₂O (1×150 mL) and brine (3×150 mL). The organic layer was dried over Na₂SO₄, filtered and concentrated in vacuo. The resulting crude material was purified via silica gel chromatography & RP column to yield the desired product.

### Step 2:

A vial with stir bar was charged with 4-bromo-1-cyclopropyl-5-methylimidazole (1.00 g, 4.97 mmol, 1.00 equiv) and tert-butyl N-{4-[(E)-2-(4,4,5,5-tetramethyl-1,3,2-dioxaborolan-2-yl)ethenyl]-1,3-thiazol-2-yl}carbamate (1.93 g, 5.47 mmol, 1.10 equiv), Pd(dtbpf)Cl₂ (648.29 mg, 1.00 mmol, 0.20 equiv), K₃PO₄ (3.17 g, 14.92 mmol, 3.00 equiv) in dioxane (30 mL) and H₂O (10 mL) under nitrogen atmosphere. The vial was capped and placed in a 90° C. bath. The reaction mixture was stirred at 90° C. for 3 h. The resulting mixture was cooled to room temperature, poured into DCM (120 mL) and washed with brine (2×80 mL). The combined organic layers were dried over Na₂SO₄, filtered and concentrated in vacuo. The resulting crude material was purified via silica gel column to yield the desired product.

### Step 3:

A vial with stir bar was charged with tert-butyl N-{4-[(E)-2-(1-cyclopropyl-5-methylimidazol-4-yl)ethenyl]-1,3-thiazol-2-yl}carbamate (780 mg, 2.25 mmol, 1.00 equiv) in DCM (4 mL), TFA (4 mL) was added. The vial was capped and placed in a 25° C. bath. The reaction mixture was stirred at 25° C. for 1 h. The resulting mixture was concentrated under vacuum, and the pH value of the solution was adjusted to 8 with sat. NaHCO₃ (aq). The aqueous layer was extracted with DCM (4×30 mL). The combined organic layer was dried over Na₂SO₄, filtered and concentrated in vacuo. The resulting crude material was purified via silica gel column to yield the desired product.

The following intermediate for compound listed in the table was prepared via a similar method:

| Compd | |
|---|---|
| # | Compound name |
| A1 | (E)-4-(2-(5-chloro-1-isopropyl-1H-imidazol-4-yl)vinyl)thiazol- |
| | 2-amine |

### Route 4:

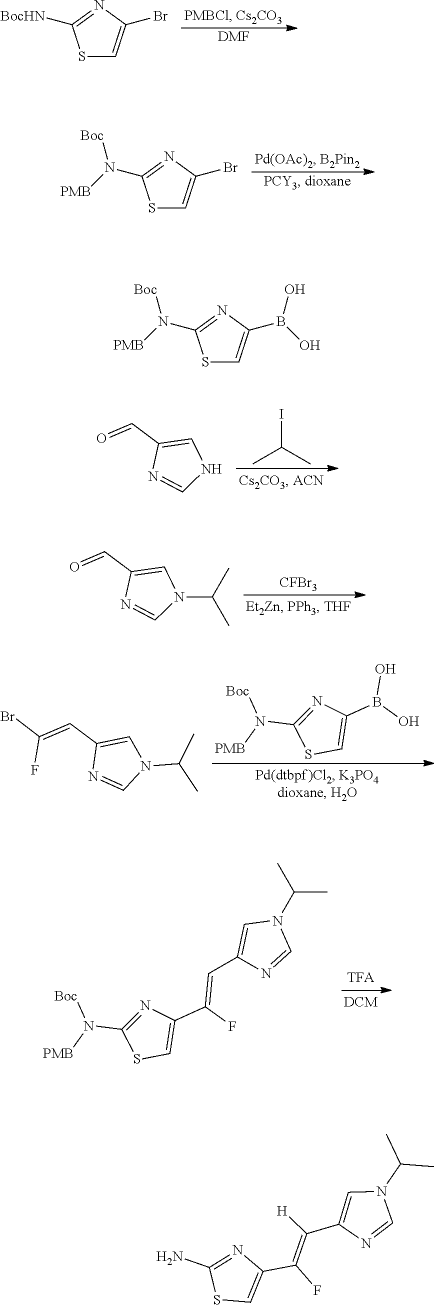

### Step 1:

A vial with stir bar was charged with tert-butyl N-(4-bromo-1,3-thiazol-2-yl)carbamate (4.00 g, 14.33 mmol, 1.00 equiv), Cs₂CO₃ (9.34 g, 28.66 mmol, 2.00 equiv), PMBCl (2.69 g, 17.20 mmol, 1.20 equiv) in DMF (60 mL, 0.24 M). The vial was evacuated and backflushed with nitrogen. The vial was capped and placed in a 70° C. bath. The reaction mixture was stirred at 70° C. for 3 h. The reaction mixture was cooled to room temperature. The reaction mixture was quenched with water. The resulting solution was extracted with EtOAc (3×200 mL) and washed with brine (3×200 mL). The organic layer was then dried over Na₂SO₄, filtered and concentrated in vacuo. The resulting crude reaction mixture was purified via silica gel chromatography to yield the desired product.

### Step 2:

A vial with stir bar was charged with tert-butyl N-(4-bromo-1,3-thiazol-2-yl)-N-[(4-methoxyphenyl)methyl]carbamate (1.40 g, 3.51 mmol, 1.00 equiv), KOAc (860 mg, 8.77 mmol, 2.50 equiv), 4,4,5,5-tetramethyl-2-(tetramethyl-1,3,2-dioxaborolan-2-yl)-1,3,2-dioxaborolane (0.98 g, 3.88 mmol, 1.10 equiv), PCy₃ (290 mg, 1.05 mmol, 0.30 equiv), Pd(OAc)₂ (160 mg, 0.70 mmol, 0.20 equiv) in dioxane (25 mL, 0.14 M). The vial was evacuated and backflushed with nitrogen. The vial was capped and placed in an 80° C. bath, and the reaction mixture was allowed to stir at 80° C. for 3 h. The reaction mixture was cooled to room temperature. The reaction mixture was poured into EtOAc (150 mL) and washed with brine (2×100 mL). The combined organic layers were dried over Na₂SO₄, filtered and concentrated in vacuo. The resulting crude material was purified via silica gel chromatography to yield the desired product.

### Step 3:

A vial with stir bar was charged with 1H-imidazole-4-carbaldehyde (5.00 g, 52.04 mmol, 1.00 equiv), 2-iodopropane (1.77.20 g, 104.07 mmol, 2.00 equiv), Cs₂CO₃ (5.09 g, 156.11 mmol, 3.00 equiv) in ACN (40 mL, 1.30 M). The vial was evacuated and backflushed with nitrogen. The vial was capped and placed in a 70° C. bath. The reaction mixture was stirred at 70° C. for 12 h. The reaction mixture was cooled to room temperature. The reaction mixture was quenched with water. The resulting solution was extracted with DCM (3×100 mL) and washed with brine (1×100 mL). The organic layer was then dried over Na₂SO₄, filtered and concentrated in vacuo. The resulting crude reaction mixture was purified via silica gel chromatography to yield the desired product.

### Step 4:

A vial with stir bar was charged with 1-isopropylimidazole-4-carbaldehyde (500 mg, 3.62 mmol, 1.00 equiv), tribromofluoromethane (1.96 g, 7.24 mmol, 2.00 equiv), PPh₃ (1.90 g, 7.24 mmol, 2.00 equiv) in THE (20 mL, 0.18 M). The vial was evacuated and backflushed with nitrogen. Diethylzinc (670.37 mg, 5.43 mmol, 1.50 equiv) was added. The vial was capped and placed in a 25° C. bath. The reaction mixture was stirred at 25° C. for 3 h. The reaction mixture was quenched with HCl (aq, 1M). The resulting solution was extracted with DCM (3×80 mL) and washed with brine (1×80 mL). The organic layer was then dried over Na₂SO₄, filtered and concentrated in vacuo. The resulting crude reaction mixture was purified via silica gel chromatography to yield the desired product.

### Step 5:

A vial with stir bar was charged with 4-[(E)-2-bromo-2-fluoroethenyl]-1-isopropylimidazole (200 mg, 0.86 mmol, 1.00 equiv), 2-[(tert-butoxycarbonyl)[(4-methoxyphenyl)methyl]amino]-1,3-thiazol-4-ylboronic acid (312.52 mg, 0.86 mmol, 1.00 equiv), K₃PO₄ (546.41 mg, 2.57 mmol, 3.00 equiv), Pd(dtbpf)Cl₂ (111.85 mg, 0.17 mmol, 0.20 equiv) in dioxane (10 mL) and H₂O (2 mL). The vial was evacuated and backflushed with nitrogen. The vial was capped and placed in a 90° C. bath. The reaction mixture was stirred at 90° C. for 3 h. The reaction mixture was cooled to room temperature. The reaction mixture was quenched with water. The resulting solution was extracted with DCM (3×50 mL) and washed with brine (1×50 mL). The organic layer was then dried over Na₂SO₄, filtered and concentrated in vacuo. The resulting crude reaction mixture was purified via RP column to yield the desired product.

### Step 6:

A vial with stir bar was charged with tert-butyl N-{4-[(Z)-1-fluoro-2-(1-isopropylimidazol-4-yl)ethenyl]-1,3-thiazol-2-yl}-N-[(4-methoxyphenyl)methyl]carbamate (700 mg, 1.48 mmol, 1.00 equiv) in TFA (5 mL, 0.30 M). The vial was evacuated and backflushed with nitrogen. The vial was capped and placed in a 70° C. bath. The reaction mixture was stirred at 70° C. for 2 h. The reaction mixture was cooled to room temperature. The resulting mixture was concentrated under vacuum, and the pH value of the solution was adjusted to 8 with sat.NaHCO₃ (aq). The aqueous layer was extracted with DCM (4×30 mL). The combined organic layer was dried over Na₂SO₄, filtered and concentrated in vacuo. The resulting crude reaction mixture was purified via silica gel chromatography to yield the desired product.

### Route 5:

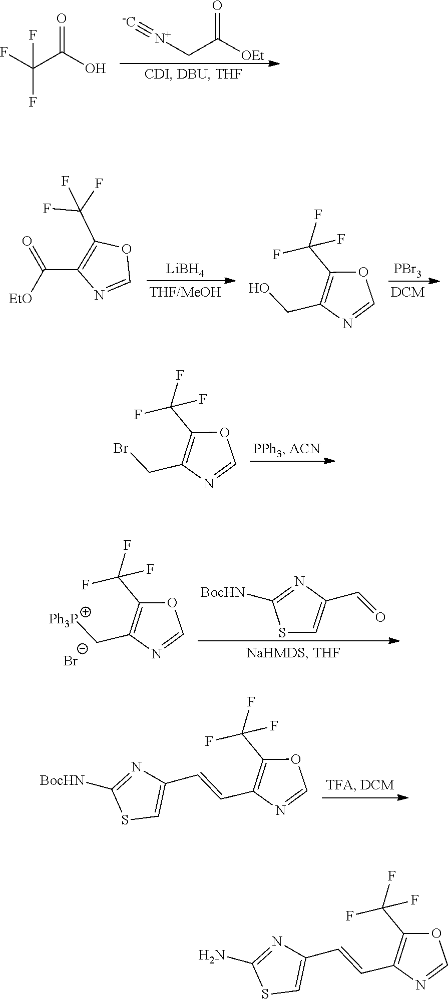

### Step 1:

A vial with stir bar was charged with TFA (2.00 g, 17.54 mmol, 1.00 equiv) and CDI (3.41 g, 21.05 mmol, 1.20 equiv) in THE (50 mL, 0.35 M). The resulting mixture was heated at 55° C. for 1 h and then cooled to 0° C. Ethyl 2-isocyanoacetate (2.38 g, 21.05 mmol, 1.20 equiv) was added in one portion, and then DBU (3.20 g, 21.05 mmol, 1.20 equiv) was added dropwise at 0° C. The vial was capped and placed in a 25° C. bath. The reaction mixture was stirred at 25° C. for 12 h. The reaction mixture was quenched with water. The resulting solution was extracted with EtOAc (3×80 mL) and washed with brine (1×80 mL). The organic layer was then dried over Na₂SO₄, filtered and concentrated in vacuo. The resulting crude reaction mixture was purified via silica gel chromatography to yield the desired product.

### Step 2:

A vial with stir bar was charged with ethyl 5-(trifluoromethyl)-1,3-oxazole-4-carboxylate (1.00 g, 4.78 mmol, 1.00 equiv) in THE (6 mL) and MeOH (3 mL), LiBH₄ (135.39 mg, 6.22 mmol, 1.30 equiv) was added in portions at 0° C. The vial was evacuated and backflushed with nitrogen. The vial was capped and placed in a 60° C. bath. The reaction mixture was stirred at 60° C. for 1 h. The reaction mixture was cooled to room temperature. The reaction was quenched with sat. NH4Cl (aq) at 0° C. The aqueous layer was extracted with DCM (3×30 mL). The organic layer was then dried over Na₂SO₄, filtered and concentrated in vacuo. The resulting crude reaction mixture was purified via silica gel chromatography to yield the desired product.

### Step 3:

A vial with stir bar was charged with [5-(trifluoromethyl)-1,3-oxazol-4-yl]methanol (1.00 g, 5.99 mmol, 1.00 equiv) in DCM (10 mL, 0.60 M), PBr₃ (3.24 g, 11.97 mmol, 2.00 equiv) was added. The vial was capped and placed in a 25° C. bath. The reaction mixture was stirred at 25° C. for 1 h. The reaction mixture was quenched with NaHCO₃ (s). The resulting mixture was filtered, the filter cake was washed with DCM (3×50 mL). The combined filtrate was concentrated under vacuum. The crude product was used in the next step without further purification.

### Step 4:

A vial with stir bar was charged with 4-(bromomethyl)-5-(trifluoromethyl)-1,3-oxazole (500 mg, 2.17 mmol, 1.00 equiv) and PPh₃ (855.36 mg, 3.26 mmol, 1.50 equiv) in ACN (8 mL, 0.27 M). The vial was evacuated and backflushed with nitrogen. The vial was capped and placed in a 70° C. bath. The reaction mixture was stirred at 70° C. for 4 h. The reaction mixture was cooled to room temperature. The resulting mixture was concentrated under vacuum. The resulting crude reaction mixture was purified via silica gel chromatography to yield the desired product.

### Step 5:

A vial with stir bar was charged with triphenyl({[5-(trifluoromethyl)-1,3-oxazol-4-yl]methyl})phosphanium bromide (517.57 mg, 1.05 mmol, 1.20 equiv) in THE (8 mL, 0.13 M), NaHMDS (2 M, 0.88 mL, 1.75 mmol, 2.00 equiv) was added dropwise at −78° C. under nitrogen atmosphere. Tert-butyl N-(4-formyl-1,3-thiazol-2-yl)carbamate (200 mg, 0.88 mmol, 1.00 equiv) in THE (2 mL) was added in portions over 10 min at −78° C. The resulting mixture was allowed to warm to 25° C. The vial was capped and placed in a 25° C. bath. The reaction mixture was stirred at 25° C. for 12 h. The reaction mixture was quenched with sat. NH₄Cl (aq). The resulting mixture was extracted with DCM (3×30 mL) and washed with brine (3×30 mL). The organic layer was then dried over Na₂SO₄, filtered and concentrated in vacuo. The resulting crude reaction mixture was purified via silica gel chromatography to yield the desired product.

### Step 6:

A vial with stir bar was charged with tert-butyl N-{4-[(E)-2-[5-(trifluoromethyl)-1,3-oxazol-4-yl]ethenyl]-1,3-thiazol-2-yl}carbamate (200 mg, 0.55 mmol, 1.00 equiv) in DCM (2 mL, 0.28 M), TFA (2 mL) was added. The vial was capped and placed in a 25° C. bath. The reaction mixture was stirred at 25° C. for 1 h. The resulting mixture was concentrated under vacuum, and the pH value of the solution was adjusted to 8 with sat.NaHCO₃ (aq). The aqueous layer was extracted with DCM (4×20 mL). The combined organic layer was dried over Na₂SO₄, filtered and concentrated in vacuo. The resulting crude material was purified via silica gel column to yield the desired product.

The following intermediates for compounds listed in the table were prepared via a similar method:

| Compound | |
|---|---|
| # | Compound name |
| B1 | (E)-(4-(2-(2-aminothiazol-4-yl)vinyl)oxazol-5-yl)methanol |
| B2 | (E)-4-(2-(4-fluoro-1-isopropyl-1H-imidazol-5- |
| | yl)vinyl)thiazol-2-amine |

### Route 6:

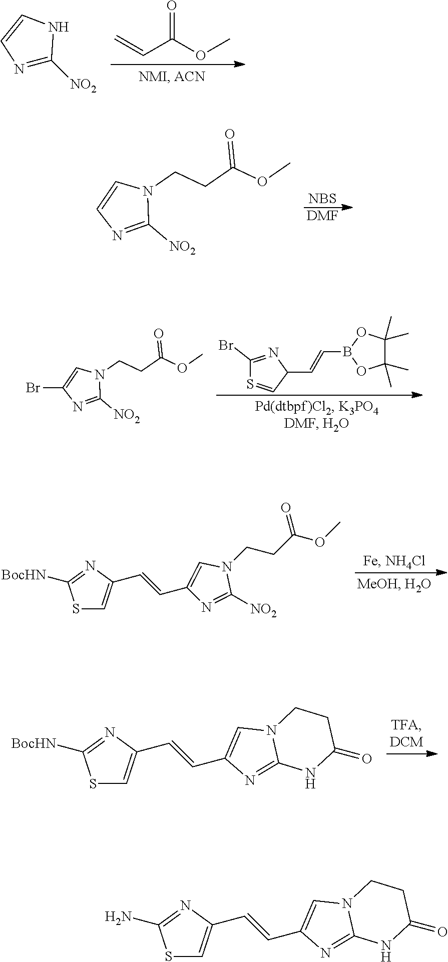

### Step 1:

A vial with stir bar was charged with 2-nitroimidazole (2.00 g, 17.69 mmol, 1.00 equiv) and methyl acrylate (3.05 g, 35.37 mmol, 2.00 equiv) in ACN (10 mL). The vial was evacuated and backflushed with nitrogen. The vial was capped and placed in a 100° C. bath. The reaction mixture was stirred at 100° C. for 12 h. The resulting mixture was concentrated under vacuum. The resulting crude material was purified via silica gel column to yield the desired product.

### Step 2:

A vial with stir bar was charged with 1-methoxy-3-(2-nitroimidazolidin-1-yl)propan-1-ol (1.50 g, 7.53 mmol, 1.00 equiv) in DMF (8 mL, 0.75 M), NBS (1.07 g, 6.03 mmol, 0.80 equiv). The vial was evacuated and backflushed with nitrogen. The vial was capped and placed in a 25° C. bath. The reaction mixture was stirred at 25° C. for 2 h. The reaction mixture was quenched with H₂O. The resulting mixture was concentrated under vacuum. The resulting crude reaction mixture was purified via RP column to yield the desired product.

### Step 3:

A vial with stir bar was charged with methyl 3-(4-bromo-2-nitroimidazol-1-yl)propanoate (400 mg, 1.44 mmol, 1.00 equiv) and tert-butyl N-{4-[(E)-2-(4,4,5,5-tetramethyl-1,3,2-dioxaborolan-2-yl)ethenyl]-1,3-thiazol-2-yl}carbamate (608.08 mg, 1.73 mmol, 1.20 equiv), Pd(dtbpf)Cl₂ (185.83 mg, 0.29 mmol, 0.20 equiv), K₃PO₄ (916.04 mg, 4.32 mmol, 3.00 equiv) in dioxane (10 mL) and H₂O (2 mL) under nitrogen atmosphere. The vial was capped and placed in a 90° C. bath. The reaction mixture was stirred at 90° C. for 2 h. The resulting mixture was cooled to room temperature, poured into DCM (80 mL) and washed with brine (2×80 mL). The combined organic layers were dried over Na₂SO₄, filtered and concentrated in vacuo. The resulting crude material was purified via silica gel column to yield the desired product.

### Step 4:

A vial with stir bar was charged with methyl 3-{4-[(E)-2-{2-[(tert-butoxycarbonyl)amino]-1,3-thiazol-4-yl}ethenyl]-2-nitroimidazol-1-yl}propanoate (400 mg, 0.95 mmol, 1.00 equiv), NH₄Cl (757.94 mg, 14.18 mmol, 15 equiv) and Fe (530 mg, 9.45 mmol, 10 equiv) in MeOH (10 mL), H₂O (2 mL). The vial was evacuated and backflushed with nitrogen. The vial was capped and placed in a 60° C. bath. The reaction mixture was stirred at 60° C. for 4 h. The resulting mixture was cooled to room temperature. The reaction mixture was quenched with H₂O. The resulting mixture was extracted with DCM (3×50 mL). The combined organic layers were washed with brine (1×50 mL). The combined organic layers were dried over Na₂SO₄, filtered and concentrated in vacuo. The resulting crude material was purified via silica gel column to yield the desired product.

### Step 5:

A vial with stir bar was charged with tert-butyl N-{4-[(E)-2-{7-oxo-5H,6H,8H-imidazo[1,2-a]pyrimidin-2-yl}ethenyl]-1,3-thiazol-2-yl}carbamate (120 mg, 0.33 mmol, 1.00 equiv) in DCM (2 mL, 0.17 M), TFA (2 mL) was added. The vial was capped and placed in a 25° C. bath. The reaction mixture was stirred at 25° C. for 1 h. The resulting mixture was concentrated under vacuum, and the pH value of the solution was adjusted to 8 with sat.NaHCO₃ (aq). The aqueous layer was extracted with DCM (4×20 mL). The combined organic layer was dried over Na₂SO₄, filtered and concentrated in vacuo. The resulting crude material was purified via silica gel column to yield the desired product.

The following intermediates for compounds listed in the table were prepared via a similar method:

| Compd | |
|---|---|
| # | Compound name |
| B23 | (E)-4-(2-(1-isopropyl-1H-imidazol-4-yl)vinyl)-5-methylthiazol- |
| | 2-amine |
| A6 | (E)-5-chloro-4-(2-(5-chloro-1-isopropyl-1H-imidazol-4- |
| | yl)vinyl)thiazol-2-amine |

### Route 7:

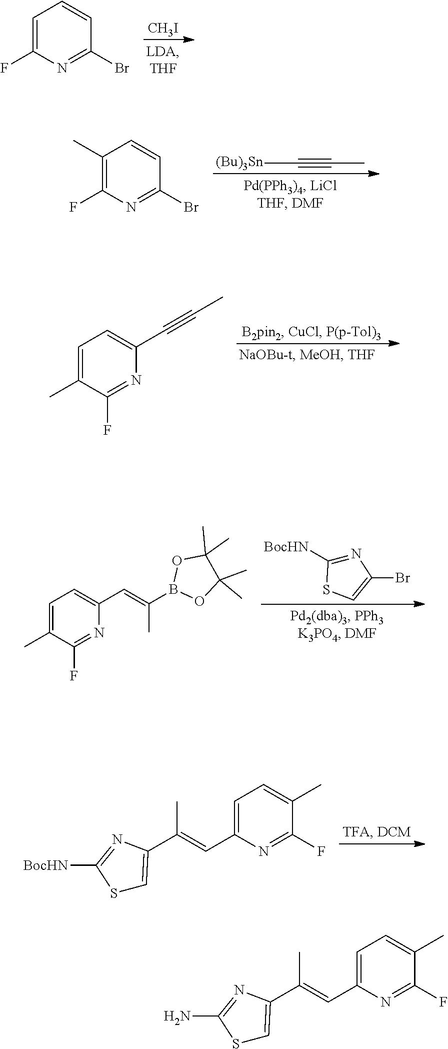

### Step 1:

A vial with stir bar was charged with 2-bromo-6-fluoropyridine (2.00 g, 11.36 mmol, 1.00 equiv) in THE (15 mL). The flask was then vacuumed and flushed with nitrogen atmosphere. LDA (2 M, 6.82 mL, 13.64 mmol, 1.20 equiv) was added dropwise over 5 min at −78° C., the mixture was stirred for 15 min at −78° C. CH₃I (1.94 g, 13.64 mmol, 1.20 equiv) in dry THE (5 mL) was added dropwise over 5 min at −78° C. The mixture slowly reached to room temperature. And the vial was capped and placed in an 25° C. bath. The reaction mixture was stirred at 25° C. overnight. The next morning, the reaction mixture was quenched with H₂O. The mixture was extracted with DCM (3×50 mL), and the combined organic layers were washed with brine (2×50 mL). The organic layer was then dried over Na₂SO₄, filtered and concentrated in vacuo. The resulting crude material was purified via silica gel column to yield the desired product.

### Step 2:

A vial with stir bar was charged with 6-bromo-2-fluoro-3-methylpyridine (1.00 g, 5.26 mmol, 1.00 equiv), LiCl (0.67 g, 15.79 mmol, 3.00 equiv), tributyl(prop-1-yn-1-yl)stannane (3.46 g, 10.53 mmol, 2.00 equiv) and Pd(PPh₃)₄ (1.22 g, 1.05 mmol, 0.20 equiv) in THE (10 mL) and DMF (10 mL). The flask was then vacuumed and flushed with nitrogen atmosphere. And the vial was capped and placed in an 80° C. bath. The reaction mixture was stirred at 80° C. for 3 h. The reaction mixture was cooled to room temperature. The reaction mixture was poured into EtOAc (200 mL) and washed with H₂O (1×100 mL), followed by brine (2×100 mL). The organic layer was then dried over Na₂SO₄, filtered and concentrated in vacuo. The resulting crude material was purified via silica gel column to yield the desired product.

### Step 3:

A vial with stir bar was charged with a solution of t-BuONa (38.66 mg, 0.40 mmol, 0.30 equiv), CuCl (26.55 mg, 0.27 mmol, 0.20 equiv), tri-p-tolylphosphine (97.94 mg, 0.32 mmol, 0.24 equiv) in THE (6.00 mL) under nitrogen atmosphere. The mixture was stirred about 30 min at room temperature. This was followed by the addition of a solution of 4,4,5,5-tetramethyl-2-(tetramethyl-1,3,2-dioxaborolan-2-yl)-1,3,2-dioxaborolane (374.52 mg, 1.48 mmol, 1.10 equiv) in THE (2 mL) at room temperature. The mixture was stirred about 10 min at room temperature. To this was added a solution of 2-fluoro-3-methyl-6-(prop-1-yn-1-yl)pyridine (200 mg, 1.34 mmol, 1.00 equiv) and MeOH (85.92 mg, 2.68 mmol, 2.00 equiv) in THE (2 mL) at room temperature. The resulting solution was stirred for 6 h at room temperature. The reaction was then quenched with water. The resulting solution was extracted with EtOAc (3×40 mL) and washed with brine (2×40 mL). The combined organic layers were dried over Na₂SO₄, filtered and concentrated in vacuo. The resulting crude material was purified via silica gel chromatography to yield the desired product.

### Step 4:

A vial with stir bar was charged with 2-fluoro-3-methyl-6-[(1Z)-2-(4,4,5,5-tetramethyl-1,3,2-dioxaborolan-2-yl)prop-1-en-1-yl]pyridine (200 mg, 0.72 mmol, 1.00 equiv), tert-butyl N-(4-bromo-1,3-thiazol-2-yl)carbamate (221.59 mg, 0.79 mmol, 1.10 equiv), K₃PO₄ (459.53 mg, 2.17 mmol, 3.00 equiv), PPh₃ (75.71 mg, 0.29 mmol, 0.40 equiv), Pd₂(dba)₃ (132.16 mg, 0.14 mmol, 0.20 equiv) and DMF (10.00 mL, 0.07 M). The flask was then vacuumed and flushed with nitrogen atmosphere. The resulting solution was stirred for 6 h at 80° C. The reaction mixture was cooled to room temperature. The reaction was then quenched with water. The resulting solution was extracted with EtOAc (3×50 mL) and washed with (3×50 mL) of brine. The organic layers were dried over Na₂SO₄, filtered and concentrated in vacuo. The resulting crude material was purified via silica gel chromatography to yield the desired product.

### Step 5:

A vial with stir bar was charged with tert-butyl N-{4-[(1E)-1-(6-fluoro-5-methylpyridin-2-yl)prop-1-en-2-yl]-1,3-thiazol-2-yl}carbamate (150 mg, 0.43 mmol, 1.00 equiv) in DCM (3 mL, 0.14 M), TFA (3 mL) was added. The vial was capped and placed in a 25° C. bath. The reaction mixture was stirred at 25° C. for 1 h. The resulting mixture was concentrated under vacuum, and the pH value of the solution was adjusted to 8 with sat.NaHCO₃ (aq). The aqueous layer was extracted with EtOAc (4×20 mL). The combined organic layer was dried over Na₂SO₄, filtered and concentrated in vacuo. The resulting crude material was purified via RP column to yield the desired product.

The following intermediate for compound listed in the table was prepared via a similar method:

| Compd | |
|---|---|
| # | Compound name |
| B24 | (E)-4-(1-(1-isopropyl-1H-imidazol-4-yl)prop-1-en-2-yl)thiazol- |
| | 2-amine |

### Route 8:

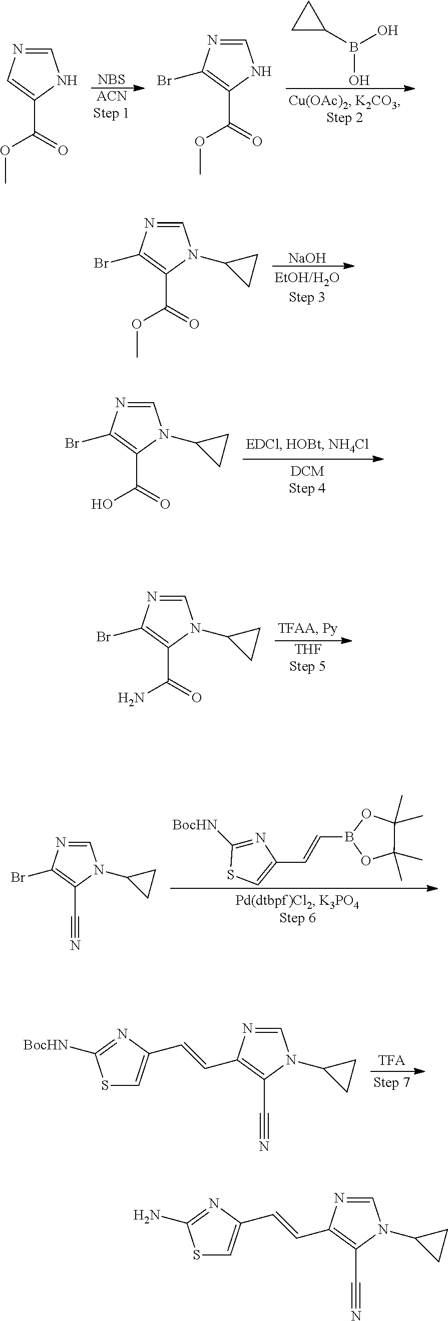

### Step 1:

Into a vial were placed methyl 3H-imidazole-4-carboxylate in ACN (300 mL), NBS (0.80 equiv) was added. The vial was evacuated and backflushed with nitrogen. The vial was capped and placed in a 25° C. bath. The reaction mixture was stirred at 25° C. for 12 h. The pH value of the residue was adjusted to 8 with saturated NaHCO₃ (aq). The resulting mixture was extracted with EtOAc (3×300 mL). The combined organic layers were washed with brine (2×200 mL). The organic layer was dried over Na₂SO₄, filtered and concentrated in vacuo. The resulting crude material was purified via silica gel chromatography to yield the desired product.

### Step 2:

Into a vial were placed methyl 5-bromo-3H-imidazole-4-carboxylate and cyclopropylboronic acid (5.00 equiv), Cu(OAc)₂ (1.00 equiv), K₂CO₃ (2.00 equiv) in DCE (300 mL). The vial was evacuated and backflushed with oxygen. The vial was capped and placed in a 70° C. bath. The reaction mixture was stirred at 70° C. for 12 h. The reaction mixture was cooled to room temperature. The reaction mixture was washed with brine (3×100 mL). The organic layer was dried over Na₂SO₄, filtered and concentrated in vacuo. The resulting crude material was purified via silica gel chromatography to yield the desired product.

### Step 3:

Into a vial were placed methyl 5-bromo-3-cyclopropylimidazole-4-carboxylate in EtOH (25.00 mL) and H₂O (25.00 mL), NaOH (2.00 equiv) was added. The vial was evacuated and backflushed with nitrogen. The vial was capped and placed in a 80° C. bath. The reaction mixture was stirred at 80° C. for 3 h. The reaction mixture was cooled to room temperature. The pH value of the mixture was adjusted to 6 with HCl (aq, 2 M). The resulting mixture was extracted with EtOAc (3×40 mL) and washed with brine (3×30 mL). The organic layer was dried over Na₂SO₄, filtered and concentrated in vacuo. The crude product was used in the next step without further purification.

### Step 4:

Into a vial were placed 5-bromo-3-cyclopropylimidazole-4-carboxylic acid, TEA (3.00 equiv), HOBt (1.20 equiv), NH₄Cl (2.00 equiv), EDCl (1.20 equiv) in DCM (50 mL). The vial was evacuated and backflushed with nitrogen. The vial was capped and placed in a 25° C. bath. The reaction mixture was stirred at 25° C. for 12 h. The reaction mixture was quenched with H₂O (30 mL). The resulting mixture was extracted with DCM (3×40 mL) and washed with brine (2×40 mL). The organic layer was dried over Na₂SO₄, filtered and concentrated in vacuo. The resulting crude material was purified via silica gel chromatography to yield the desired product.

### Step 5:

Into a vial were placed 5-bromo-3-cyclopropylimidazole-4-carboxamide and Py (3.00 equiv) in THE (50 mL), TFAA (1.50 equiv) was added. The vial was evacuated and backflushed with nitrogen. The vial was capped and placed in a 25° C. bath. The reaction mixture was stirred at 25° C. for 3 h. The pH value of the residue was adjusted to 8 with saturated NaHCO₃ (aq). The resulting mixture was extracted with DCM (3×30 mL) and was washed with brine (3×30 mL). The organic layer was dried over Na₂SO₄, filtered and concentrated in vacuo. The resulting crude material was purified via silica gel chromatography to yield the desired product.

### Step 6:

Into a vial were placed 5-bromo-3-cyclopropylimidazole-4-carbonitrile and tert-butyl N-{4-[(E)-2-(4,4,5,5-tetramethyl-1,3,2-dioxaborolan-2-yl)ethenyl]-1,3-thiazol-2-yl}carbamate (1.20 equiv), K₃PO₄ (4.00 equiv) and Pd(dtbpf)Cl₂ (0.20 equiv) in dioxane (30 mL) and H₂O (6 mL). The vial was evacuated and backflushed with nitrogen. The vial was capped and placed in a 90° C. bath. The reaction mixture was stirred at 90° C. for 3 h. The reaction mixture was cooled to room temperature. The resulting mixture was diluted with water (40 mL). The resulting mixture was extracted with DCM (3×40 mL) and washed with brine (3×40 mL). The organic layer was dried over Na₂SO₄, filtered and concentrated in vacuo. The resulting crude material was purified via silica gel chromatography to yield the desired product.

### Step 7:

Into a vial were placed tert-butyl N-{4-[(E)-2-(5-cyano-1-cyclopropylimidazol-4-yl)ethenyl]-1,3-thiazol-2-yl}carbamate in DCM (10 ml), TFA (10 mL) was added. The vial was evacuated and backflushed with nitrogen. The vial was capped and placed in a 25° C. bath. The reaction mixture was stirred at 25° C. for 1 h. The resulting solution was concentrated in vacuo. The pH value of the residue was adjusted to 8 with saturated NaHCO₃ (aq). The resulting mixture was extracted with DCM (3×30 mL) and was washed with brine (1×30 mL). The organic layer was dried over Na₂SO₄, filtered and concentrated in vacuo. The crude product was used in the next step without further purification.

### Route 9:

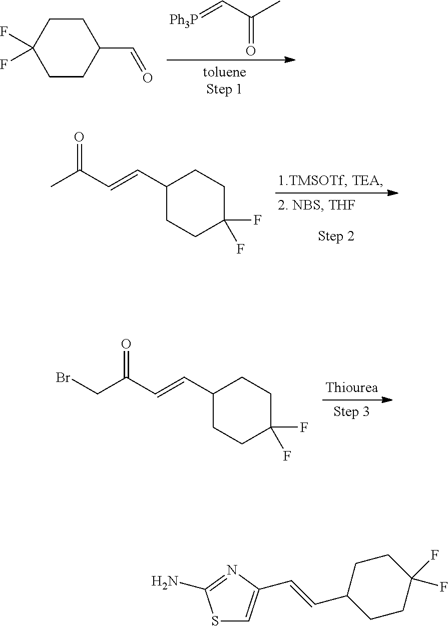

### Step 1:

Into a vial were placed 4,4-difluorocyclohexane-1-carbaldehyde and 1-(triphenyl-lambda5-phosphanylidene)propan-2-one in toluene (10 mL). The vial was evacuated and backflushed with nitrogen. The vial was capped and placed in a 110° C. bath. The reaction mixture was stirred at 110° C. for 12 h. The resulting mixture was concentrated under reduced pressure. The resulting crude material was purified via silica gel chromatography to yield the desired product.

### Step 2:

Into a vial were placed (3E)-4-(4,4-difluorocyclohexyl)but-3-en-2-one and TEA (2.50 equiv) in toluene (10.00 mL), TMSOTf (1.5 equiv) was added at 0° C. The vial was evacuated and backflushed with nitrogen. The vial was capped and placed in a 25° C. bath. The reaction mixture was stirred at 25° C. for 1 h. The reaction was quenched with NaHCO₃ (aq). The resulting mixture was extracted with EtOAc (3×20 mL) and washed with brine (3×10 mL). The organic layer was dried over Na₂SO₄, filtered and concentrated in vacuo. The crude product was used in the next step without further purification.

Into another vial were placed (E)-((4-(4,4-difluorocyclohexyl)buta-1,3-dien-2-yl)oxy)trimethylsilane and NaHCO₃ (1.50 equiv) in THE (10 mL), NBS (1.10 equiv) was added. The vial was evacuated and backflushed with nitrogen. The vial was capped and placed in a 25° C. bath. The reaction mixture was stirred at 25° C. for 1 h. The reaction was quenched with NaHCO₃(aq). The resulting mixture was extracted with EtOAc (3×20 mL) and washed with brine (3×10 mL). The organic layer was dried over Na₂SO₄, filtered and concentrated in vacuo. The crude product was used in the next step without further purification.

### Step 3:

Into a vial were placed (3E)-1-bromo-4-(4,4-difluorocyclohexyl)but-3-en-2-one and thiourea (5.27 equiv) in EtOH (15 mL). The vial was evacuated and backflushed with nitrogen. The vial was capped and placed in a 70° C. bath. The reaction mixture was stirred at 70° C. for 1 h. The pH value of the residue was adjusted to 8 with saturated NaHCO₃ (aq). The resulting mixture was extracted with DCM (3×30 mL) and washed with brine (1×20 mL). The organic layer was dried over Na₂SO₄, filtered and concentrated in vacuo. The resulting crude material was purified via silica gel chromatography to yield the desired product.

### Route 10:

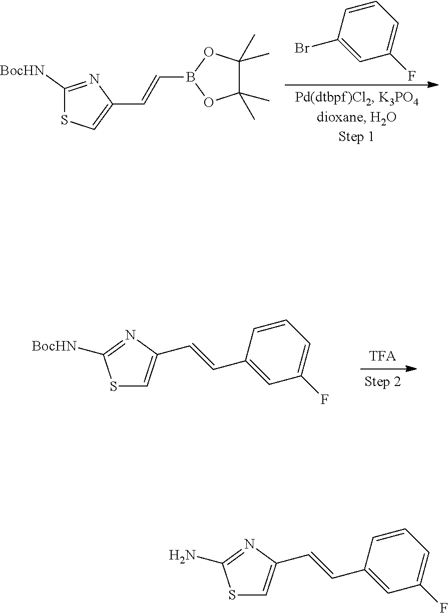

### Step 1:

Into a vial were placed tert-butyl N-{4-[(E)-2-(4,4,5,5-tetramethyl-1,3,2-dioxaborolan-2-yl)ethenyl]-1,3-thiazol-2-yl}carbamate and 1-bromo-3-fluorobenzene (1.10 equiv), Pd(dtbpf)Cl₂ (0.20 equiv), K₃PO₄ (3.00 equiv) in dioxane (15.00 mL) and H₂O (3.00 mL). The vial was evacuated and backflushed with nitrogen. The vial was capped and placed in a 90° C. bath. The reaction mixture was stirred at 90° C. for 2 h. The reaction mixture was cooled to room temperature. The resulting mixture was diluted with water (30 mL). The resulting mixture was extracted with DCM (3×30 mL). The combined organic layers were washed with brine (3×30 mL). The organic layer was dried over Na₂SO₄, filtered and concentrated in vacuo. The resulting crude material was purified via silica gel chromatography to yield the desired product.

### Step 2:

Into a vial were placed tert-butyl N-{4-[(E)-2-(3-fluorophenyl)ethenyl]-1,3-thiazol-2-yl}carbamate in DCM (2 mL), TFA (2 mL) was added. The vial was evacuated and backflushed with nitrogen. The vial was capped and placed in a 25° C. bath. The reaction mixture was stirred at 25° C. for 1 h. The resulting solution was concentrated in vacuo. The pH value of the residue was adjusted to 8 with saturated NaHCO₃ (aq). The resulting mixture was extracted with DCM (3×30 mL) and washed with brine (3×30 mL). The organic layer was dried over Na₂SO₄, filtered and concentrated in vacuo. The resulting crude material was used directly for next step.

The intermediate (E)-4-(2-(benzofuran-5-yl)vinyl)thiazol-2-amine was made in a similar manner.

### Route 11:

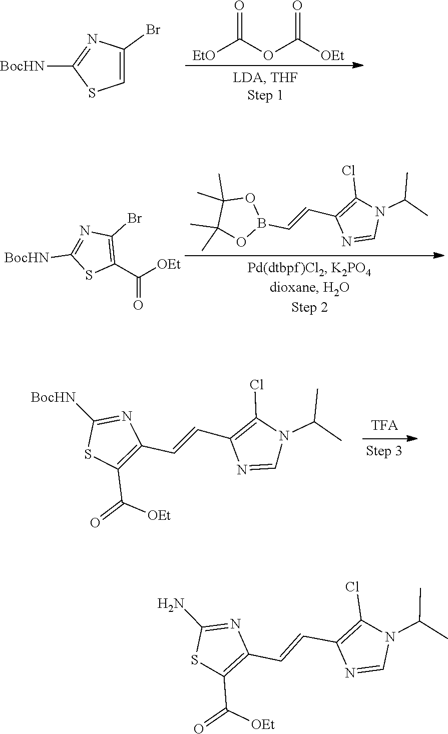

### Step 1:

Into a vial were placed tert-butyl N-(5-bromo-1,3-thiazol-2-yl)carbamate in THE (20 mL), LDA (3.35 equiv, 2 M) was added dropwise at 0° C., diethyl pyrocarbonate (3.30 equiv) was added dropwise at 0° C. The vial was evacuated and backflushed with nitrogen. The vial was capped and placed in a 25° C. bath. The reaction mixture was stirred at 25° C. for 12 h. The resulting solution was quenched with H₂O (20 mL). The resulting mixture was extracted with EtOAc (3×30 mL) and washed with brine (3×30 mL). The organic layer was dried over Na₂SO₄, filtered and concentrated in vacuo. The resulting crude material was purified via silica gel chromatography to yield the desired product.

### Step 2:

Into a vial were placed ethyl 4-bromo-2-[(tert-butoxycarbonyl)amino]-1,3-thiazole-5-carboxylate, K₃PO₄ (3.00 equiv), 5-chloro-1-isopropyl-4-[(E)-2-(4,4,5,5-tetramethyl-1,3,2-dioxaborolan-2-yl)ethenyl]imidazole (1.10 equiv) and Pd(dtbpf)Cl₂ (0.10 equiv) in dioxane (50 mL) and water (10 mL). The vial was evacuated and backflushed with nitrogen. The vial was capped and placed in a 70° C. bath. The reaction mixture was stirred at 70° C. for 1 h. The reaction mixture was cooled to room temperature. The resulting mixture was diluted with water (30 mL). The resulting mixture was extracted with EtOAc (3×40 mL) and washed with brine (3×40 mL). The organic layer was dried over Na₂SO₄, filtered and concentrated in vacuo. The resulting crude material was purified via silica gel chromatography to yield the desired product.

### Step 3:

Into a vial were placed ethyl 4-[(E)-2-(5-chloro-1-isopropylimidazol-4-yl)ethenyl]-2-(methylamino)-1,3-thiazole-5-carboxylate in DCM (3 mL), TFA (3 mL) was added. The vial was evacuated and backflushed with nitrogen. The vial was capped and placed in a 25° C. bath. The reaction mixture was stirred at 25° C. for 1 h. The resulting mixture was concentrated in vacuo. The mixture was adjusted pH to 8 with saturated NaHCO₃ (aq.). The aqueous layer was extracted with DCM (2×20 mL). The organic layer was dried over Na₂SO₄, filtered and concentrated in vacuo. The resulting crude material was used directly for next step.

### Carboxylic Acid Synthesis:

### Route 1:

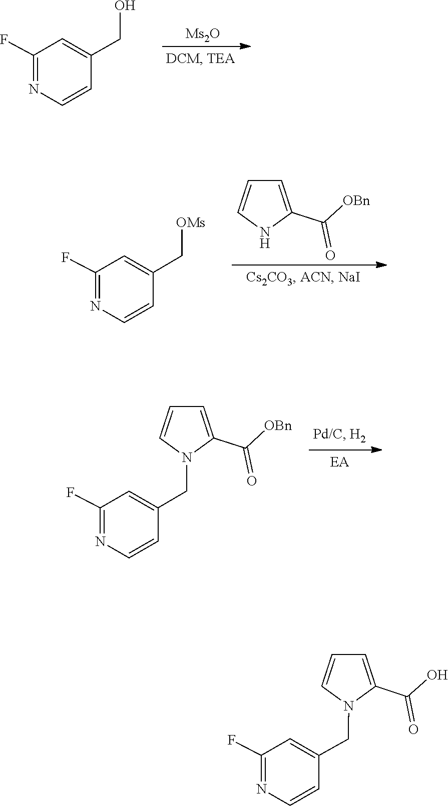 [SMILES: "Ms2O" ; OCc1ccnc(F)c1 ; CS(=O)(=O)OCc1ccnc(F)c1 ; O=C(OCc1ccccc1)c1ccc[nH]1 ; O=C(OCc1ccccc1)c1cccn1Cc1ccnc(F)c1 ; O=C(O)c1cccn1Cc1ccnc(F)c1]

### Step 1:

A vial with stir bar was charged with (2-fluoropyridin-4-yl)methanol (4.50 g, 35.40 mmol, 1.00 equiv) and Et₃N (5.33 g, 52.67 mmol, 1.50 equiv) in DCM (100.00 mL, 0.35 M), Ms₂O (7.34 g, 42.14 mmol, 1.10 equiv) was added at 0° C. The vial was capped and placed in a 25° C. bath. The reaction mixture was stirred at 25° C. for 1 h. The reaction mixture was quenched with water. The resulting mixture was extracted with DCM (3×100 mL) and washed with brine (1×100 mL). The combined organic layer was then dried over Na₂SO₄, filtered and concentrated in vacuo. The crude precipitated material was used in the next step without further purification.

### Step 2:

A vial with stir bar was charged with (2-fluoropyridin-4-yl)methyl methanesulfonate (6.00 g, 29.24 mmol, 1.00 equiv), benzyl 1H-pyrrole-2-carboxylate (4.71 g, 23.39 mmol, 0.80 equiv), Cs₂CO₃ (19.05 g, 58.48 mmol, 2.00 equiv), NaI (0.44 g, 2.924 mmol, 0.10 equiv) in ACN (150.00 mL, 0.20 M) under nitrogen atmosphere. The vial was capped and placed in a 60° C. bath. The reaction mixture was stirred at 60° C. for 4 h. The reaction mixture was cooled to room temperature. The reaction mixture was quenched with water. The resulting solution was extracted with EtOAc (3×100 mL) and the combined organic layers were washed with brine (2×100 mL). The organic layer was then dried over Na₂SO₄, filtered and concentrated in vacuo. The resulting crude material was purified via silica gel column to yield the desired product.

### Step 3:

A vial with stir bar was charged with benzyl 1-[(2-fluoropyridin-4-yl)methyl]pyrrole-2-carboxylate (2.00 g, 6.45 mmol, 1.00 equiv) and Pd/C (10%, 2.00 g, 18.83 mmol, 2.92 equiv) in EtOAc (20 mL, 0.32 M) under nitrogen atmosphere. The flask was then vacuumed and flushed with hydrogen. The reaction mixture was hydrogenated at room temperature for 2 h under hydrogen atmosphere using a hydrogen balloon. Then the reaction mixture was filtered through a celite pad and the filtrate was concentrated under reduced pressure. The resulting crude material was purified via RP column to yield the desired product.

### Route 2:

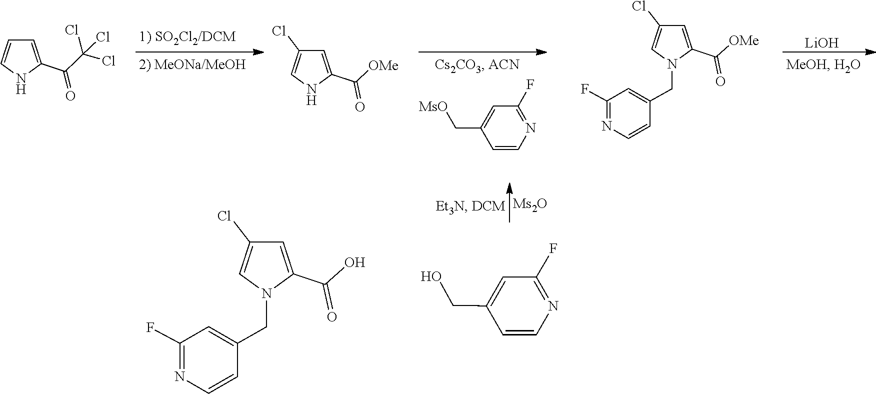

### Step 1:

A vial with stir bar was charged with 2,2,2-trichloro-1-(1H-pyrrol-2-yl)ethanone (3.00 g, 14.12 mmol, 1.00 equiv) in DCM (50 mL, 0.28 M) under nitrogen atmosphere, sulfonyl chloride (1.33 g, 9.89 mmol, 0.70 equiv) was added at 0° C. The vial was capped and placed in a 0° C. bath. The reaction mixture was stirred at 0° C. for 2 h. The reaction was quenched with sat. NH₄Cl (aq). The resulting mixture was extracted with DCM (3×200 mL). The organic layers were washed with brine (2×100 mL). The combined organic layer was then dried over Na₂SO₄, filtered, and concentrated in vacuo. The resulting crude material was purified via RP column to yield the desired product.

### Step 2:

A vial with stir bar was charged with 2,2,2-trichloro-1-(4-chloro-1H-pyrrol-2-yl)ethanone (1.00 g, 4.05 mmol, 1.00 equiv) and sodium methoxide (0.66 g, 12.15 mmol, 3.00 equiv) in MeOH (20 mL, 0.20 M) under nitrogen atmosphere. The vial was capped and placed in a 25° C. bath. The reaction mixture was stirred at 25° C. for 1 h. The reaction mixture was poured into DCM (200 mL) and washed with brine (2×150 mL). The combined organic layer was then dried over Na₂SO₄, filtered and concentrated in vacuo. The resulting crude material was purified via silica gel column to yield the desired product.

### Step 3:

A vial with stir bar was charged with methyl 4-chloro-1H-pyrrole-2-carboxylate (1.1 g, 6.89 mmol, 1.00 equiv) and Cs₂CO₃ (6.76 g, 20.75 mmol, 3.00 equiv) in ACN (30 mL, 0.23 M) under nitrogen atmosphere, (2-fluoropyridin-4-yl)methyl methanesulfonate (1.70 g, 8.27 mmol, 1.20 equiv) was added. The vial was capped and placed in a 60° C. bath. The reaction mixture was stirred at 60° C. for 2 h. The reaction mixture was cooled to room temperature. The reaction mixture was poured into H₂O (150 mL). The resulting mixture was extracted with DCM (3×150 mL) and the combined organic layers were washed with brine (3×100 mL). The combined organic layer was then dried over Na₂SO₄, filtered and concentrated in vacuo. The resulting crude material was purified via RP column to yield the desired product.

### Step 4:

A vial with stir bar was charged with methyl 4-chloro-1-[(2-fluoropyridin-4-yl)methyl]pyrrole-2-carboxylate (2.00 g, 7.44 mmol, 1.00 equiv) in MeOH (15 mL) and H₂O (5 mL) under nitrogen atmosphere, LiOH·H₂O (3.13 g, 74.59 mmol, 10.02 equiv) was added. The vial was capped and placed in a 25° C. bath. The reaction mixture was stirred at 25° C. for 3 h. The volatile solvents were removed in vacuo. The pH value of the solution was adjusted to 7 with HCl (aq, 1 M), and the precipitate was filtered and washed with H₂O (2×7 mL). The crude precipitated material was used in the next step without further purification.

### Preparation of (2-fluoropyridin-4-yl)methyl methanesulfonate

A vial with stir bar was charged with (2-fluoropyridin-4-yl)methanol (4.50 g, 35.40 mmol, 1.00 equiv) and Et₃N (5.33 g, 52.67 mmol, 1.50 equiv) in DCM (50.00 mL), Ms₂O (7.34 g, 42.14 mmol, 1.10 equiv) was added at 0° C. The vial was capped and placed in a 25° C. bath. The reaction mixture was stirred at 25° C. for 1 h. The reaction mixture was quenched with water. The resulting mixture was extracted with DCM (3×100 mL) and the combined organic layers were washed with brine (1×100 mL). The combined organic layer was then dried over Na₂SO₄, filtered and concentrated in vacuo. The crude precipitated material was used in the next step without further purification.

### Route 3:

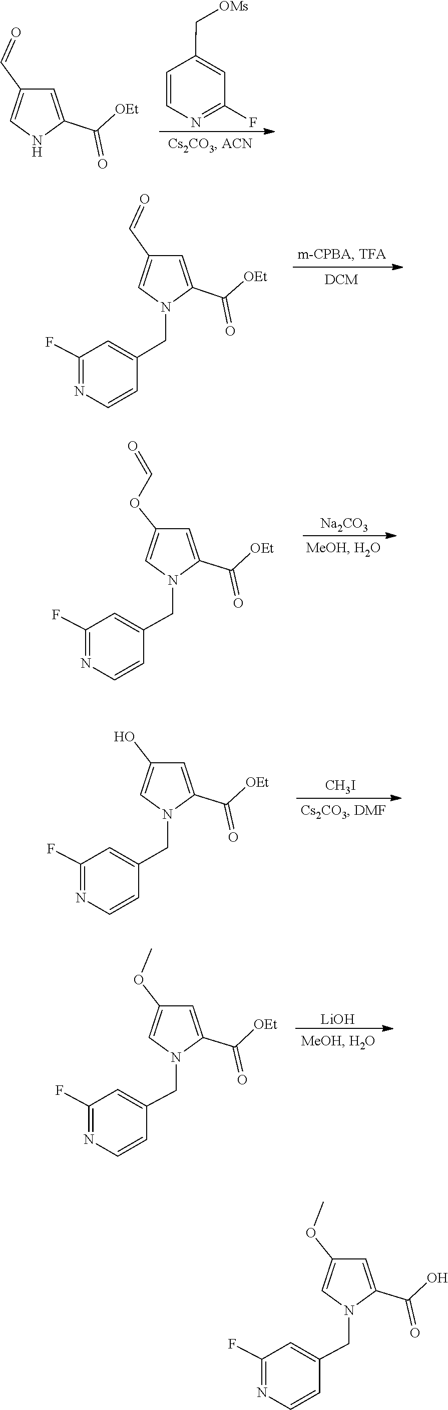

### Step 1:

A vial with stir bar was charged with ethyl 4-formyl-1H-pyrrole-2-carboxylate (3.00 g, 17.95 mmol, 1.00 equiv) and Cs₂CO₃ (1.75 g, 53.84 mmol, 3.00 equiv) in ACN (60 mL, 0.30 M) under nitrogen atmosphere, (2-fluoropyridin-4-yl)methyl methanesulfonate (4.42 g, 21.54 mmol, 1.20 equiv) was added. The vial was capped and placed in a 50° C. bath. The reaction mixture was stirred at 50° C. for 1 h. The reaction mixture was cooled to room temperature. The resulting mixture was diluted with DCM (200 mL) and washed with (3×100 mL) of brine. The combined organic layer was then dried over Na₂SO₄, filtered and concentrated in vacuo. The resulting crude material was purified via silica gel column to yield the desired product.

### Step 2:

A vial with stir bar was charged with ethyl 1-[(2-fluoropyridin-4-yl)methyl]-4-formylpyrrole-2-carboxylate (2.00 g, 7.24 mmol, 1.00 equiv), TFA (82.55 mg, 0.72 mmol, 0.10 equiv) and m-CPBA (1.87 g, 10.86 mmol, 1.50 equiv) in DCM (40 mL, 0.18 M) under nitrogen atmosphere. The vial was capped and placed in a 25° C. bath. The reaction mixture was stirred at 25° C. for 3 h. The reaction was quenched by the addition of Na₂S₂O₃ (aq) (40 mL). The resulting mixture was extracted with DCM (2×40 mL). The combined organic layers were washed with Na₂S₂O₃ (aq, 20 mL). The combined organic layer was then dried over Na₂SO₄, filtered and concentrated in vacuo. The resulting crude material was purified via silica gel column to yield the desired product.

### Step 3:

A vial with stir bar was charged with ethyl 1-[(2-fluoropyridin-4-yl)methyl]-4-(formyloxy)pyrrole-2-carboxylate (280 mg, 0.96 mmol, 1.00 equiv) in MeOH (6 mL) and H₂O (2 mL) under nitrogen atmosphere, Na₂CO₃ (123.01 mg, 1.15 mmol, 1.20 equiv) was added. The vial was capped and placed in a 25° C. bath. The reaction mixture was stirred at 25° C. for 30 min. The resulting mixture was diluted with EtOAc (50 mL) and washed with (2×30 mL) of brine. The combined organic layer was then dried over Na₂SO₄, filtered and concentrated in vacuo. The crude was used in the next step without further purification.

### Step 4:

A vial with stir bar was charged with ethyl 1-[(2-fluoropyridin-4-yl)methyl]-4-hydroxypyrrole-2-carboxylate (200.00 mg, 0.76 mmol, 1.00 equiv), Cs₂CO₃ (739.78 mg, 2.27 mmol, 3.00 equiv) in DMF (10 mL, 0.08 M) under nitrogen atmosphere, methyl iodide (322.28 mg, 2.27 mmol, 3.00 equiv) was added. The vial was capped and placed in a −30° C. bath. The reaction mixture was stirred at −30° C. for 30 min. And then the resulting mixture was stirred for 2 h at room temperature. The reaction mixture was quenched with H₂O. The resulting mixture was extracted with EtOAc (3×40 mL) and washed with brine (3×40 mL). The combined organic layer was then dried over Na₂SO₄, filtered and concentrated in vacuo. The resulting crude material was purified via silica gel column to yield the desired product.

### Step 5:

A vial with stir bar was charged with ethyl 1-[(2-fluoropyridin-4-yl)methyl]-4-methoxypyrrole-2-carboxylate (2.00 g, 7.19 mmol, 1.00 equiv) and LiOH (1.72 g, 71.87 mmol, 10.00 equiv) in EtOH (8 mL) and H₂O (2 mL) under nitrogen atmosphere. The vial was capped and placed in a 40° C. bath. The reaction mixture was stirred at 40° C. for 6 h. The reaction mixture was cooled to room temperature. The volatile solvents were removed in vacuo. The pH value of the solution was adjusted to 7 with HCl (1 mol/L), and the precipitate was filtered and washed with H₂O (2×8 mL). The crude precipitated material was used in the next step without further purification.

### Route 4:

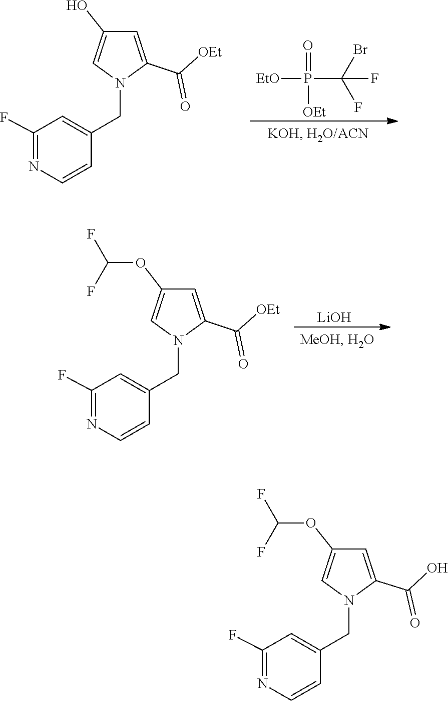

### Step 1:

A vial with stir bar was charged with ethyl 1-[(2-fluoropyridin-4-yl)methyl]-4-hydroxypyrrole-2-carboxylate (50 mg, 0.19 mmol, 1.00 equiv) in ACN (5 mL) and H₂O (5 mL), KOH (116.77 mg, 2.08 mmol, 11 equiv) under nitrogen atmosphere. The vial was capped and placed in a −78° C. bath. Diethyl bromodifluoromethylphosphonate (202.08 mg, 0.76 mmol, 4.00 equiv) dropwise at −78° C. The reaction mixture was allowed to warm to room temperature, and stirred at room temperature for 12 h. The reaction mixture was diluted with DCM (30 mL) and washed with brine (1×30 mL). The organic layer was then dried over Na₂SO₄, filtered and concentrated in vacuo. The resulting crude material was purified via prep-TLC to yield the desired product.

### Step 2:

A vial with stir bar was charged with methyl 4-(difluoromethoxy)-1-[(2-fluoropyridin-4-yl)methyl]pyrrole-2-carboxylate (210 mg, 0.70 mmol, 1.00 equiv) and LiOH (167.52 mg, 6.99 mmol, 10 equiv) in MeOH (8 mL) and H₂O (2 mL) under nitrogen atmosphere. The vial was capped and placed in a 40° C. bath. The reaction mixture was stirred at 40° C. for 4 h. The reaction mixture was cooled to room temperature. The pH value of the solution was adjusted to 7 with HCl (1 M), The resulting mixture was extracted with DCM (3×40 mL) and washed with brine (1×40 mL). The combined organic layer was then dried over Na₂SO₄, filtered and concentrated in vacuo. The resulting crude material was purified via RP column to yield the desired product.

### Route 5:

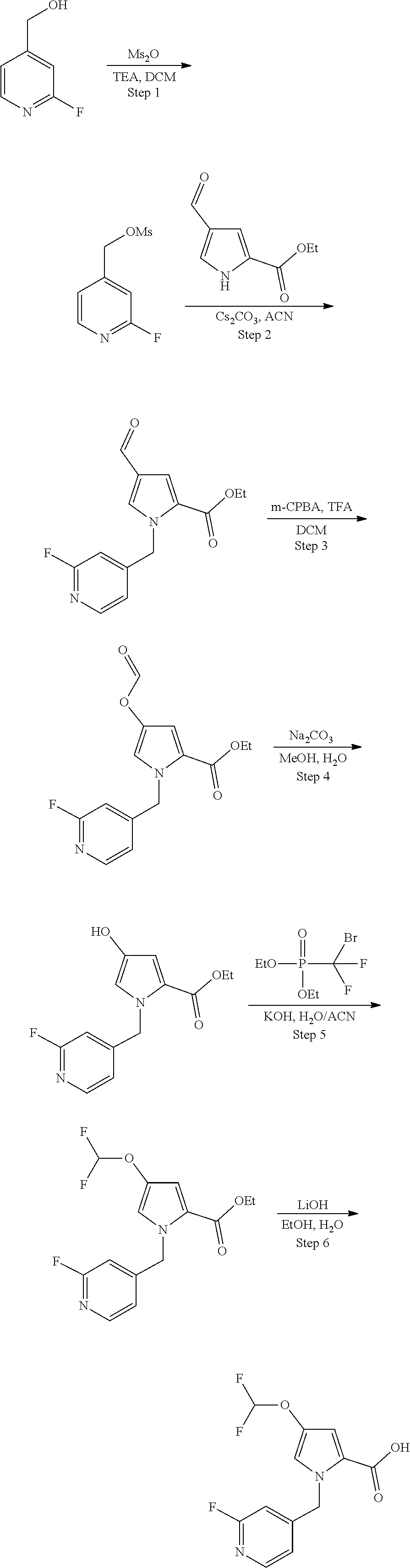

### Step 1:

Into a vial were placed (2-fluoropyridin-4-yl)methanol and TEA (3.00 equiv) in DCM (100 mL), methanesulfonic anhydride (1.20 equiv) was added. The vial was evacuated and backflushed with nitrogen. The vial was capped and placed in a 25° C. bath. The reaction mixture was stirred at 25° C. for 1 h. The resulting solution was quenched with H₂O (100 mL) and extracted with DCM (3×100 mL). The resulting mixture were washed with brine (2×50 mL). The organic layer was dried over Na₂SO₄, filtered and concentrated in vacuo. The resulting crude material was used directly for next step.

### Step 2:

Into a vial were placed ethyl 4-formyl-1H-pyrrole-2-carboxylate and (2-fluoropyridin-4-yl) methyl methanesulfonate (1.20 equiv) in MeCN (30 mL), Cs₂CO₃ (3.00 equiv) was added at room temperature. The vial was evacuated and backflushed with nitrogen. The vial was capped and placed in a 25° C. bath. The reaction mixture was stirred at 25° C. for 30 min. The resulting solution was quenched with H₂O (20 mL) and extracted with DCM (3×20 mL). The combined organic layers were washed with brine (3×20 mL). The organic layer was dried over Na₂SO₄, filtered and concentrated in vacuo. The resulting crude material was purified via silica gel chromatography to yield the desired product.

### Step 3:

Into a vial were placed ethyl 1-[(2-fluoropyridin-4-yl)methyl]-4-formylpyrrole-2-carboxylate and TFA (0.10 equiv) in DCM (30 mL) at 0° C., m-CPBA (2.00 equiv) was added. The vial was evacuated and backflushed with nitrogen. The vial was capped and placed in a 25° C. bath. The reaction mixture was stirred at 25° C. for 2 h. The resulting solution was quenched with Na₂S₂O₄ (aq) at 0° C. The resulting mixture was extracted with DCM (3×30 mL) and washed with brine (3×30 mL). The organic layer was dried over Na₂SO₄, filtered and concentrated in vacuo. The resulting crude material was purified via silica gel chromatography to yield the desired product.

### Step 4:

Into a vial were placed ethyl 1-[(2-fluoropyridin-4-yl)methyl]-4-(formyloxy)pyrrole-2-carboxylate and Na₂CO₃ (1.20 equiv) in MeOH (12 mL) and H₂O (4 mL). The vial was evacuated and backflushed with nitrogen. The vial was capped and placed in a 25° C. bath. The reaction mixture was stirred at 25° C. for 30 min. The resulting solution was quenched with H₂O (10 mL) and extracted with DCM (3×10 mL). The combined organic layers were washed with brine (3×10 mL). The organic layer was dried over Na₂SO₄, filtered and concentrated in vacuo. The resulting crude material was purified via silica gel chromatography to yield the desired product.

### Step 5:

Into a vial were placed ethyl 1-[(2-fluoropyridin-4-yl)methyl]-4-hydroxypyrrole-2-carboxylate and KOH (11.00 equiv) in ACN (30 mL) and H₂O (40 mL), and then diethyl bromodifluoromethylphosphonate (4.00 equiv) in ACN (20 mL) was added dropwise at −30° C. The vial was capped and placed in a 25° C. bath. The reaction mixture was stirred at 25° C. for 12 h. The resulting solution was quenched with H₂O (40 mL). The resulting mixture was extracted with DCM (3×40 mL) and washed with brine (2×40 mL). The organic layer was dried over Na₂SO₄, filtered and concentrated in vacuo. The resulting crude material was purified via silica gel chromatography to yield the desired product.

### Step 6:

Into a mL vial was placed ethyl 4-(difluoromethoxy)-1-[(2-fluoropyridin-4-yl)methyl]pyrrole-2-carboxylate in H₂O (3 mL) and EtOH (10 mL), LiOH (3.00 equiv) was added. The vial was evacuated and backflushed with nitrogen. The vial was capped and placed in a 25° C. bath. The reaction mixture was stirred at 25° C. for 12 h. The pH value of the mixture was adjusted to 6 with HCl (2 M). The resulting mixture was extracted with DCM (3×20 mL) and washed with brine (3×10 mL). The organic layer was dried over Na₂SO₄, filtered and concentrated in vacuo. The crude product was used in the next step without further purification.

The following intermediates were prepared in a similar manner.

Compound name

4-(difluoromethoxy)-1-((2-fluoropyridin-4-yl)methyl-d2)-1H-pyrrole-2-carboxylic acid
1-((2-fluoropyridin-4-yl)methyl)-4-hydroxy-1H-pyrrole-2-carboxylic acid
4-ethoxy-1-((2-fluoropyridin-4-yl)methyl)-1H-pyrrole-2-carboxylic acid
4-(fluoromethoxy)-1-((2-fluoropyridin-4-yl)methyl)-1H-pyrrole-2-carboxylic acid
1-((2,3-difluoropyridin-4-yl)methyl)-4-methoxy-1H-pyrrole-2-carboxylic acid
1-((2,3-difluoropyridin-4-yl)methyl)-4-(methoxy-d3)-1H-pyrrole-2-carboxylic acid
4-(difluoromethoxy)-1-((2-fluoro-5-methylpyridin-4-yl)methyl)-1H-pyrrole-2-carboxylic acid
4-(difluoromethoxy)-1-((2-fluoro-3-methylpyridin-4-yl)methyl)-1H-pyrrole-2-carboxylic acid
4-(difluoromethoxy)-1-((2-fluoro-3,5-dimethylpyridin-4-yl)methyl)-1H-pyrrole-2-carboxylic acid

### Route 6

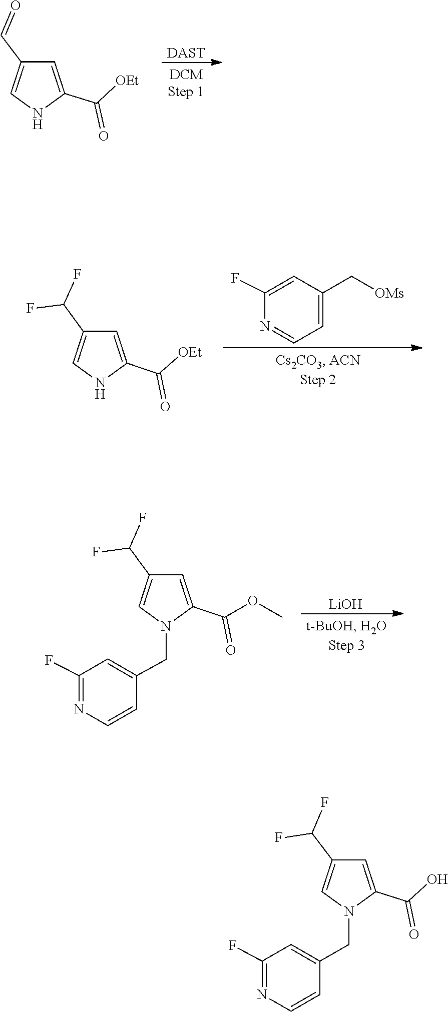

### Step 1:

Into a vial were placed ethyl 4-formyl-1H-pyrrole-2-carboxylate in DCM (8 mL), DAST (2 mL) was added. The vial was evacuated and backflushed with nitrogen. The vial was capped and placed in a 25° C. bath. The reaction mixture was stirred at 25° C. for 12 h. The reaction mixture was quenched with H₂O (30 mL). The resulting mixture was extracted with DCM (3×20 mL) and washed with brine (2×20 mL). The organic layer was dried over Na₂SO₄, filtered and concentrated in vacuo. The resulting crude material was purified via silica gel chromatography to yield the desired product.

### Step 2:

Into a mL vial were placed with ethyl 4-(difluoromethyl)-1H-pyrrole-2-carboxylate and (2-fluoropyridin-4-yl)methyl methanesulfonate (1.20 equiv) in ACN (15 mL), Cs₂CO₃ (2.00 equiv) was added. The vial was evacuated and backflushed with nitrogen. The vial was capped and placed in a 60° C. bath. The reaction mixture was stirred at 60° C. for 12 h. The reaction mixture was cooled to room temperature. The reaction mixture was quenched with H₂O (30 mL). The resulting solution was extracted with DCM (3×30 mL) and washed with brine (3×30 mL). The organic layer was dried over Na₂SO₄, filtered and concentrated in vacuo. The resulting crude material was purified via silica gel chromatography to yield the desired product.

### Step 3:

Into a vial were placed ethyl 4-(difluoromethyl)-1-[(2-fluoropyridin-4-yl)methyl]pyrrole-2-carboxylate and LiOH (5.00 equiv) in t-BuOH (6 mL) and H₂O (2 mL). The vial was evacuated and backflushed with nitrogen. The vial was capped and placed in a 40° C. bath. The reaction mixture was stirred at 40° C. for 12 h. The reaction mixture was cooled to room temperature. The pH value of the mixture was adjusted to 6 with HCl (aq, 2 M). The resulting mixture was extracted with EtOAc (3×30 mL). The organic layer was dried over Na₂SO₄, filtered and concentrated in vacuo. The resulting crude material was purified via silica gel chromatography to yield the desired product.

The intermediate 1-((2-fluoropyridin-4-yl)methyl)-4-(trifluoromethyl)-1H-pyrrole-2-carboxylic acid was prepared in a similar manner.

### Route 7:

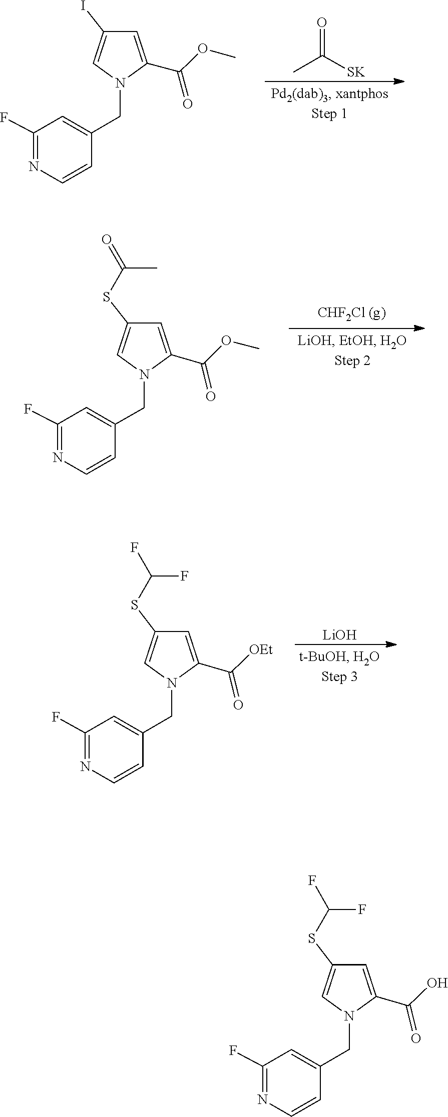

### Step 1:

Into a vial were placed methyl 1-[(2-fluoropyridin-4-yl)methyl]-4-iodopyrrole-2-carboxylate and 1-(potassiosulfanyl)ethanone (4.00 equiv) in acetone (30 mL) and toluene (60 mL) xantphos (0.40 equiv), Pd₂(dba)₃ (0.20 equiv) was added. The vial was evacuated and backflushed with nitrogen. The vial was capped and placed in a 80° C. bath. The reaction mixture was stirred at 80° C. for 5 h. The reaction mixture was cooled to room temperature. The resulting solution was quenched with H₂O (50 mL). The resulting solution was extracted with DCM (3×50 mL) and washed with brine (3×50 mL). The organic layer was dried over Na₂SO₄, filtered and concentrated in vacuo. The resulting crude material was purified via silica gel chromatography to yield the desired product.

### Step 2:

Into a vial were placed methyl 4-[(difluoromethyl)sulfanyl]-1-[(2-fluoropyridin-4-yl)methyl]pyrrole-2-carboxylate and LiOH (5.00 equiv) in EtOH (6 ml) and H₂O (1 ml), CHF₂Cl (g) was bubbled through the mixture for 1 h at 40° C. The reaction mixture was cooled to room temperature. The resulting solution was quenched with H₂O (50 mL). The resulting solution was extracted with DCM (3×50 mL) and washed with brine (3×50 mL). The organic layer was dried over Na₂SO₄, filtered and concentrated in vacuo. The resulting crude material was purified via silica gel chromatography to yield the desired product.

### Step 3:

Into a vial were placed methyl 4-[(difluoromethyl)sulfanyl]-1-[(2-fluoropyridin-4-yl)methyl]pyrrole-2-carboxylate, LiOH (5.00 equiv) in t-BuOH (6 ml) and H₂O (3 ml). The vial was evacuated and backflushed with nitrogen. The vial was capped and placed in a 40° C. bath. The reaction mixture was stirred at 40° C. for 5 h. The reaction mixture was cooled to room temperature. The pH value of the mixture was adjusted to 6 with HCl (aq, 2 M). The precipitated solid was collected by filtration and washed with H₂O (2×10 mL). The precipitated solid was evaporated under vacuum. The crude product was used in the next step without further purification.

### Route 8

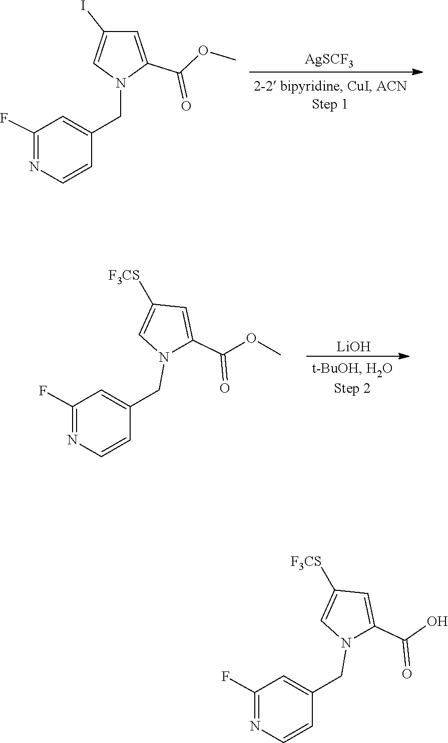

### Step 1:

Into a 20-mL sealed tube, were placed methyl 1-[(2-fluoropyridin-4-yl)methyl]-4-iodopyrrole-2-carboxylate (200 mg, 0.55 mmol, 1.00 equiv) and 2,2′-bipyridine (173.48 mg, 1.11 mmol, 2.00 equiv) in ACN (10 ml), silver (trifluoromethyl)sulfanide (348.11 mg, 1.66 mmol, 3.00 equiv) and CuI (211.54 mg, 1.11 mmol, 2.00 equiv) was added. The sealed tube was evacuated and backflushed with nitrogen. The sealed tube was capped and placed in a 100° C. bath. The reaction mixture was stirred at 100° C. for 4 h. The reaction mixture was cooled to room temperature. The resulting solution was quenched with H₂O (50 mL). The resulting solution was extracted with DCM (3×50 mL) and washed with brine (3×50 mL). The organic layer was dried over Na₂SO₄, filtered and concentrated in vacuo. The resulting crude material was purified via silica gel chromatography to yield the desired product.

### Step 2:

Into a 50-mL vial, were placed methyl 1-[(2-fluoropyridin-4-yl)methyl]-4-[(trifluoromethyl)sulfanyl]pyrrole-2-carboxylate (100 mg, 0.30 mmol, 1.00 equiv) in t-BuOH (4 ml) and H₂O (2 ml), LiOH (35.82 mg, 1.50 mmol, 5.00 equiv) was added. The vial was evacuated and backflushed with nitrogen. The vial was capped and placed in a 40° C. bath. The reaction mixture was stirred at 40° C. for 5 h. The reaction mixture was cooled to room temperature. The pH value of the mixture was adjusted to 6 with HCl (aq, 2 M). The precipitated solid was collected by filtration and washed with H₂O (2×5 mL). The precipitated solid was evaporated under vaccum. The crude product was used in the next step without further purification.

### Final Compounds Synthesis

### Route 1:

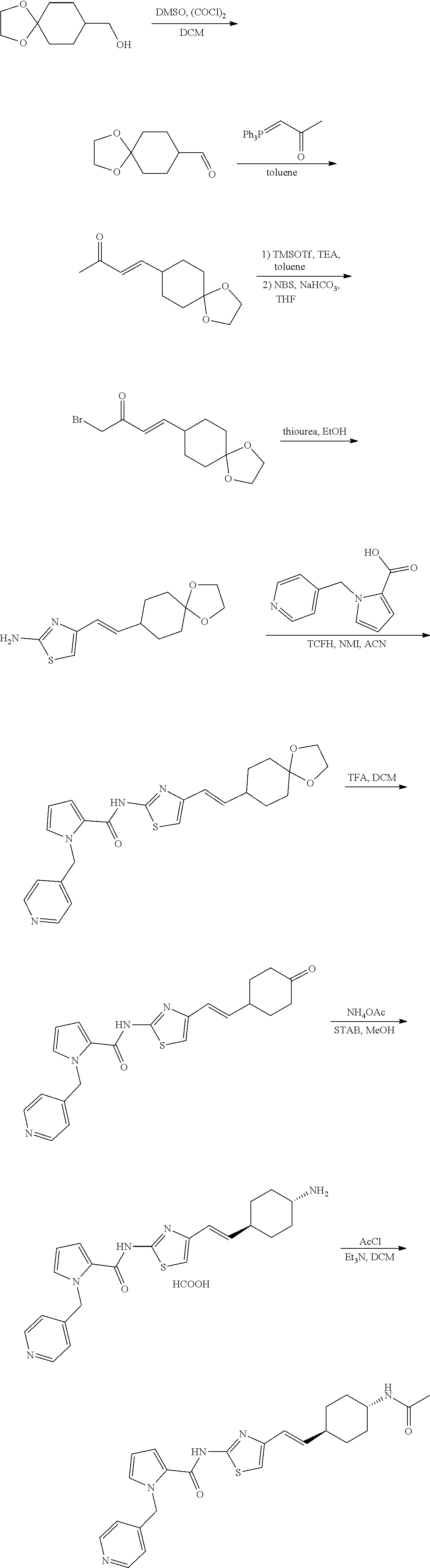

### Step 1:

A vial with stir bar was charged with oxalyl chloride (1.00 mL, 11.74 mmol, 2.02 equiv) in DCM (10 mL), DMSO (2 mL, 28.16 mmol, 4.85 equiv) in DCM (5 mL) was added slowly at −78° C. under nitrogen atmosphere. Upon completion of the addition, the mixture was stirred at −78° C. for 5 min, followed by addition of a solution of the 1,4-dioxaspiro[4.5]decan-8-ylmethanol (1.00 g, 5.81 mmol, 1.00 equiv) in DCM (5 mL) was added dropwise at −78° C. The resulting mixture was stirred for 1 h at −78° C. Then TEA (4.00 mL, 28.78 mmol, 4.96 equiv) was added dropwise at −78° C. The resulting mixture was allowed to warm to 0° C. and stirred at 0° C. for 30 min. The resulting mixture was diluted with DCM (200 mL). The resulting mixture was washed with (3×50 mL) of brine. The combined organic layer was dried over Na₂SO₄, filtered and concentrated in vacuo. The resulting crude material was used directly for next step without further purification.

### Step 2:

A vial with stir bar was charged with 1,4-dioxaspiro[4.5]decane-8-carbaldehyde (1.00 g, 5.88 mmol, 1.00 equiv), 1-(triphenyl-lambda5-phosphanylidene)propan-2-one (4.11 g, 12.93 mmol, 2.20 equiv) in toluene (25.00 mL, 0.24 M) under nitrogen atmosphere. The vial was capped and placed in a 110° C. bath. The reaction mixture was stirred at 110° C. overnight. The reaction mixture was cooled to room temperature and concentrated under vacuum. The resulting crude material was purified via silica gel chromatography to yield the desired product.

### Step 3:

A vial with stir bar was charged with (3E)-4-{1,4-dioxaspiro[4.5]decan-8-yl}but-3-en-2-one (1.00 g, 4.76 mmol, 1.00 equiv), TEA (721.87 mg, 7.13 mmol, 1.50 equiv) in toluene (12.00 mL) under nitrogen atmosphere, TMSOTf (1.37 g, 6.18 mmol, 1.30 equiv) in toluene (4 mL) was added at 0° C. The vial was capped and placed in a 0° C. bath. The reaction mixture was stirred at 0° C. for 2 h. The reaction mixture was then quenched with NaHCO₃(aq). The resulting solution was extracted with EtOAc (3×50 mL) and washed with brine (2×50 mL). The organic layers were dried over Na₂SO₄, filtered and concentrated in vacuo. A 100 mL vial with stir bar was charged with the crude product from the previous step in THE (10.00 mL, 0.35 M) under nitrogen atmosphere, NBS (693.15 mg, 3.89 mmol, 1.10 equiv) was added. The vial was capped and placed in a 0° C. bath. The reaction mixture was stirred at 0° C. for 30 min. The resulting mixture was then quenched with NaHCO₃(aq), the resulting solution was extracted with EtOAc (3×40 mL) and washed with brine (2×40 mL), and the organic layers were dried over Na₂SO₄, filtered and concentrated in vacuo. The resulting crude material was used directly for next step.

### Step 4:

A vial with stir bar was charged with (3E)-1-bromo-4-{1,4-dioxaspiro[4.5]decan-8-yl}but-3-en-2-one (1.00 g, 3.46 mmol, 1.00 equiv), thiourea (526.48 mg, 6.92 mmol, 2.00 equiv) in EtOH (20.00 mL, 0.17 M) under nitrogen atmosphere. The vial was capped and placed in a 70° C. bath. The reaction mixture was stirred at 70° C. for 2 h. The reaction mixture was cooled to room temperature and concentrated under vacuum. The reaction mixture was then quenched with NaHCO₃(aq). The resulting solution was extracted with DCM (3×40 mL) and washed with brine (2×40 mL). The combined organic layers were dried over Na₂SO₄, filtered and concentrated in vacuo. The resulting crude material was purified via silica gel chromatography to yield the desired product.

### Step 5:

A vial with stir bar was charged with 4-[(E)-2-{1,4-dioxaspiro[4.5]decan-8-yl}ethenyl]-1,3-thiazol-2-amine (200 mg, 0.75 mmol, 1.00 equiv) and 1-(pyridin-4-ylmethyl)pyrrole-2-carboxylic acid (151.83 mg, 0.75 mmol, 1.00 equiv), NMI (215.78 mg, 2.63 mmol, 3.50 equiv) in ACN (6 mL, 0.08 M) under nitrogen atmosphere, TCFH (252.81 mg, 0.90 mmol, 1.20 equiv) was added. The vial was capped and placed in a 50° C. bath. The reaction mixture was stirred at 50° C. for 8 h. The reaction mixture was cooled to room temperature. The reaction mixture was poured into DCM (50 mL) and washed with brine (2×50 mL), and the combined organic layers were dried over Na₂SO₄, filtered and concentrated in vacuo. The resulting crude material was purified via silica gel chromatography to yield the desired product.

### Step 6:

A vial with stir bar was charged with N-{4-[(E)-2-{1,4-dioxaspiro[4.5]decan-8-yl}ethenyl]-1,3-thiazol-2-yl}-1-(pyridin-4-ylmethyl)pyrrole-2-carboxamide (500 mg, 1.11 mmol, 1.00 equiv) in DCM (3 mL, 0.11 M), TFA (3 mL) was added. The vial was capped and placed in a 25° C. bath. The reaction mixture was stirred at 25° C. for 1 h. The resulting mixture was concentrated under vacuum, and the pH value of the solution was adjusted to 7 with sat.NaHCO₃ (aq). The aqueous layer was extracted with DCM (3×30 mL) and washed with brine (2×30 mL). The combined organic layer was dried over Na₂SO₄, filtered and concentrated in vacuo. The resulting crude material was purified via silica gel column to yield the desired product.

### Step 7:

A vial with stir bar was charged with N-{4-[(E)-2-(4-oxocyclohexyl)ethenyl]-1,3-thiazol-2-yl}-1-(pyridin-4-ylmethyl)pyrrole-2-carboxamide (100 mg, 0.25 mmol, 1.00 equiv), NH₄OAc (75.85 mg, 0.98 mmol, 4.00 equiv) in MeOH (8 mL, 0.03 M), STAB (104.28 mg, 0.49 mmol, 2.00 equiv) was added. The vial was capped and placed in a 25° C. bath. The reaction mixture was stirred at 25° C. for 4 h. The reaction mixture was then quenched with water. The resulting solution was extracted with DCM (3×30 mL) and washed with brine (1×30 mL). The combined organic layers were dried over Na₂SO₄, filtered and concentrated in vacuo. The resulting crude material was purified via RP column to yield the desired product.

### Step 8:

A vial with stir bar was charged with N-{4-[(E)-2-(4-aminocyclohexyl)ethenyl]-1,3-thiazol-2-yl}-1-(pyridin-4-ylmethyl)pyrrole-2-carboxamide (150 mg, 0.37 mmol, 1.00 equiv), Et₃N (111.74 mg, 1.10 mmol, 3.00 equiv) in DCM (5.00 mL), acetyl chloride (34.54 mg, 0.44 mmol, 1.20 equiv) was added at 0° C. The vial was capped and placed in a 25° C. bath. The reaction mixture was stirred at 25° C. for 1 h. The reaction mixture was poured into DCM (25 mL) and washed with brine (1×20 mL). The organic layer was then dried over Na₂SO₄, filtered and concentrated in vacuo. The resulting crude material was purified via RP column to yield the desired product B17. LC-MS (ES⁺): [M+1]⁺=450.

The following compounds were prepared via a similar method:

| | Observed | |
|---|---|---|
| | molecular | |
| | ion | Compound name |
| B9 | 461 | 1-(pyridin-4-ylmethyl)-N-(4-((E)-2-((1s,4s)-4- |
| | | (trifluoromethyl)cyclohexyl)vinyl)thiazol-2-yl)-1H-pyrrole-2- |
| | | carboxamide |
| B10 | 461 | 1-(pyridin-4-ylmethyl)-N-(4-((E)-2-((1r,4r)-4- |
| | | (trifluoromethyl)cyclohexyl)vinyl)thiazol-2-yl)-1H-pyrrole-2- |
| | | carboxamide |
| B11 | 422 | N-(4-((E)-2-((1s,4s)-4-(methylamino)cyclohexyl)vinyl)thiazol-2-yl)-1- |
| | | (pyridin-4-ylmethyl)-1H-pyrrole-2-carboxamide |
| B12 | 422 | N-(4-((E)-2-((1r,4r)-4-(methylamino)cyclohexyl)vinyl)thiazol-2-yl)-1- |
| | | (pyridin-4-ylmethyl)-1H-pyrrole-2-carboxamide |
| B15 | 450 | N-(4-((E)-2-((1s,4s)-4-acetamidocyclohexyl)vinyl)thiazol-2-yl)-1- |
| | | (pyridin-4-ylmethyl)-1H-pyrrole-2-carboxamide |
| B16 | 408 | N-(4-((E)-2-((1s,4s)-4-aminocyclohexyl)vinyl)thiazol-2-yl)-1-(pyridin-4- |
| | | ylmethyl)-1H-pyrrole-2-carboxamide |

### Route 3:

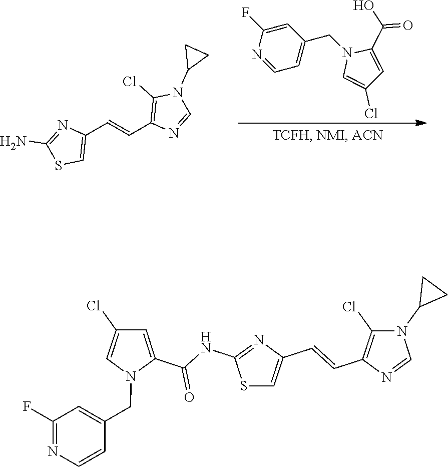 [SMILES: Nc1nc(/C=C/c2ncn(C3CC3)c2Cl)cs1 ; O=C(O)c1cc(Cl)cn1Cc1ccnc(F)c1 ; O=C(Nc1nc(/C=C/c2ncn(C3CC3)c2Cl)cs1)c1cc(Cl)cn1Cc1ccnc(F)c1]

A vial with stir bar was charged with (E)-4-(2-(5-chloro-1-cyclopropyl-1H-imidazol-4-yl)vinyl)thiazol-2-amine (50.00 mg, 0.187 mmol, 1.00 equiv), 4-chloro-1-((2-fluoropyridin-4-yl)methyl)-1H-pyrrole-2-carboxylic acid (48 mg, 0.187 mmol, 1.00 equiv), NMI (54 mg, 0.65 mmol, 3.50 equiv) in ACN (1.0 mL, 0.187 M) under nitrogen atmosphere. TCFH (60 mg, 0.215 mmol, 1.15 equiv) was added, and the vial was capped and placed in a 50° C. bath. The reaction mixture was stirred at 50° C. for 4 h. The reaction mixture was cooled to room temperature. The reaction mixture was poured into EtOAc (6 mL) and washed with brine (1×5 mL). The combined organic layers were dried over Na₂SO₄, filtered and concentrated in vacuo. The resulting crude material was purified via silica gel chromatography to yield the desired product A12. LC-MS (ES⁺): [M+1]⁺=503.

The following compounds were prepared via a similar method:

| | Observed | |
|---|---|---|
| | molecular | |
| | ion | Compound name |
| A2 | 501 | (E)-N-(4-(2-(5-chloro-1-isopropyl-1H-imidazol-4-yl)vinyl)thiazol-2-yl)-1-((2- |
| | | fluoropyridin-4-yl)methyl)-4-methoxy-1H-pyrrole-2-carboxamide |
| A3 | 497 | (E)-4-chloro-1-((2-fluoropyridin-4-yl)methyl)-N-(4-(2-(1-(trifluoromethyl)-1H- |
| | | imidazol-4-yl)vinyl)thiazol-2-yl)-1H-pyrrole-2-carboxamide |
| A4 | 507 | (E)-4-chloro-N-(4-(2-(1-(1,1-difluoropropan-2-yl)-1H-imidazol-4- |
| | | yl)vinyl)thiazol-2-yl)-1-((2-fluoropyridin-4-yl)methyl)-1H-pyrrole-2- |
| | | carboxamide |
| A5 | 479 | (E)-4-chloro-N-(4-(2-(1-(difluoromethyl)-1H-imidazol-4-yl)vinyl)thiazol-2-yl)- |
| | | 1-((2-fluoropyridin-4-yl)methyl)-1H-pyrrole-2-carboxamide |
| A6 | 535 | (E)-N-(5-chloro-4-(2-(5-chloro-1-isopropyl-1H-imidazol-4-yl)vinyl)thiazol-2- |
| | | yl)-1-((2-fluoropyridin-4-yl)methyl)-4-methoxy-1H-pyrrole-2-carboxamide |
| A7 | 537 | (E)-N-(4-(2-(5-chloro-1-isopropyl-1H-imidazol-4-yl)vinyl)thiazol-2-yl)-4- |
| | | (difluoromethoxy)-1-((2-fluoropyridin-4-yl)methyl)-1H-pyrrole-2- |
| | | carboxamide |
| A8 | 498 | (E)-4-chloro-1-((2-fluoropyridin-4-yl)methyl)-N-(4-(2-(5- |
| | | (trifluoromethyl)oxazol-4-yl)vinyl)thiazol-2-yl)-1H-pyrrole-2-carboxamide |
| A9 | 485 | (Z)-N-(4-(1-fluoro-2-(1-isopropyl-1H-imidazol-4-yl)vinyl)thiazol-2-yl)-1-((2- |
| | | fluoropyridin-4-yl)methyl)-4-methoxy-1H-pyrrole-2-carboxamide |
| A10 | 539 | (E)-4-chloro-1-((2-fluoropyridin-4-yl)methyl)-N-(4-(2-(1-isopropyl-5- |
| | | (trifluoromethyl)-1H-imidazol-4-yl)vinyl)thiazol-2-yl)-1H-pyrrole-2- |
| | | carboxamide |
| A11 | 505 | (E)-4-chloro-N-(4-(2-(5-chloro-1-cyclopropyl-1H-imidazol-4-yl)vinyl)thiazol- |
| | | 2-yl)-1-((2-fluoropyridin-4-yl)methyl-d2)-1H-pyrrole-2-carboxamide |
| A13 | 499 | (E)-N-(4-(2-(5-chloro-1-cyclopropyl-1H-imidazol-4-yl)vinyl)thiazol-2-yl)-1- |
| | | ((2-fluoropyridin-4-yl)methyl)-4-methoxy-1H-pyrrole-2-carboxamide |
| A14 | 501 | (E)-N-(4-(2-(5-chloro-1-cyclopropyl-1H-imidazol-4-yl)vinyl)thiazol-2-yl)-1- |
| | | ((2-fluoropyridin-4-yl)methyl-d2)-4-methoxy-1H-pyrrole-2-carboxamide |
| A15 | 535 | (E)-N-(4-(2-(5-chloro-1-cyclopropyl-1H-imidazol-4-yl)vinyl)thiazol-2-yl)-4- |
| | | (difluoromethoxy)-1-((2-fluoropyridin-4-yl)methyl)-1H-pyrrole-2- |
| | | carboxamide |
| A16 | 537 | (E)-N-(4-(2-(5-chloro-1-cyclopropyl-1H-imidazol-4-yl)vinyl)thiazol-2-yl)-4- |
| | | (difluoromethoxy)-1-((2-fluoropyridin-4-yl)methyl-d2)-1H-pyrrole-2- |
| | | carboxamide |
| A17 | 513 | (E)-N-(4-(2-(5-chloro-1-cyclopropyl-1H-imidazol-4-yl)vinyl)thiazol-2-yl)-4- |
| | | ethoxy-1-((2-fluoropyridin-4-yl)methyl)-1H-pyrrole-2-carboxamide |
| B1 | 426 | (E)-1-((2-fluoropyridin-4-yl)methyl)-N-(4-(2-(5-(hydroxymethyl)oxazol-4- |
| | | yl)vinyl)thiazol-2-yl)-1H-pyrrole-2-carboxamide |
| B2 | 455 | (E)-N-(4-(2-(4-fluoro-1-isopropyl-1H-imidazol-5-yl)vinyl)thiazol-2-yl)-1-((2- |
| | | fluoropyridin-4-yl)methyl)-1H-pyrrole-2-carboxamide |
| B3 | 464 | (E)-1-((2-fluoropyridin-4-yl)methyl)-N-(4-(2-(7-oxo-5,6,7,8- |
| | | tetrahydroimidazo[1,2-a]pyrimidin-2-yl)vinyl)thiazol-2-yl)-1H-pyrrole-2- |
| | | carboxamide |
| B4 | 472 | (E)-N-(4-(2-(5-(difluoromethoxy)pyridin-2-yl)vinyl)thiazol-2-yl)-1-((2- |
| | | fluoropyridin-4-yl)methyl)-1H-pyrrole-2-carboxamide |
| B5 | 474 | (E)-1-((2-fluoropyridin-4-yl)methyl)-N-(4-(2-(5-(trifluoromethyl)pyridin-2- |
| | | yl)vinyl)thiazol-2-yl)-1H-pyrrole-2-carboxamide |
| B6 | 479 | (E)-N-(4-(4-cyclopropoxy-2-fluorostyryl)thiazol-2-yl)-1-((2-fluoropyridin-4- |
| | | yl)methyl)-1H-pyrrole-2-carboxamide |
| B7 | 452 | (E)-N-(4-(1-(6-fluoro-5-methylpyridin-2-yl)prop-1-en-2-yl)thiazol-2-yl)-1-((2- |
| | | fluoropyridin-4-yl)methyl)-1H-pyrrole-2-carboxamide |
| B8 | 440 | (E)-N-(4-(2-(5-chloropyridin-2-yl)vinyl)thiazol-2-yl)-1-((2-fluoropyridin-4- |
| | | yl)methyl)-1H-pyrrole-2-carboxamide |
| B13 | 431 | (E)-N-(4-(2-(5-cyanopyridin-2-yl)vinyl)thiazol-2-yl)-1-((2-fluoropyridin-4- |
| | | yl)methyl)-1H-pyrrole-2-carboxamide |
| B14 | 445 | (E)-N-(4-(2-(5-cyano-6-methylpyridin-2-yl)vinyl)thiazol-2-yl)-1-((2- |
| | | fluoropyridin-4-yl)methyl)-1H-pyrrole-2-carboxamide |
| B18 | 408 | N-(4-((E)-2-((1r,4r)-4-aminocyclohexyl)vinyl)thiazol-2-yl)-1-(pyridin-4- |
| | | ylmethyl)-1H-pyrrole-2-carboxamide |
| B19 | 421 | (E)-N-(4-(2-(5-cyanooxazol-4-yl)vinyl)thiazol-2-yl)-1-((2-fluoropyridin-4- |
| | | yl)methyl)-1H-pyrrole-2-carboxamide |
| B20 | 462 | (E)-N-(4-(2-(5-cyano-1-isopropyl-1H-imidazol-4-yl)vinyl)thiazol-2-yl)-1-((2- |
| | | fluoropyridin-4-yl)methyl)-1H-pyrrole-2-carboxamide |
| B21 | 438 | E)-1-((2-fluoropyridin-4-yl)methyl)-N-(4-(2-(1-isopropyl-1H-imidazol-4-yl-2- |
| | | d)vinyl)thiazol-2-yl)-1H-pyrrole-2-carboxamide |
| B22 | 399 | (E)-1-((2-fluoropyridin-4-yl)methyl)-N-(4-(2-(tetrahydrofuran-3- |
| | | yl)vinyl)thiazol-2-yl)-1H-pyrrole-2-carboxamide |
| B23 | 451 | (E)-1-((2-fluoropyridin-4-yl)methyl)-N-(4-(2-(1-isopropyl-1H-imidazol-4- |
| | | yl)vinyl)-5-methylthiazol-2-yl)-1H-pyrrole-2-carboxamide |
| B24 | 451 | (E)-1-((2-fluoropyridin-4-yl)methyl)-N-(4-(1-(1-isopropyl-1H-imidazol-4- |
| | | yl)prop-1-en-2-yl)thiazol-2-yl)-1H-pyrrole-2-carboxamide |
| B25 | 433 | (E)-N-(4-(1-(1-isopropyl-1H-imidazol-4-yl)prop-1-en-2-yl)thiazol-2-yl)-1- |
| | | (pyridin-4-ylmethyl)-1H-pyrrole-2-carboxamide |
| B26 | 397 | (E)-N-(4-(2-cyclopentylvinyl)thiazol-2-yl)-1-((2-fluoropyridin-4-yl)methyl)- |
| | | 1H-pyrrole-2-carboxamide |

### Route 4:

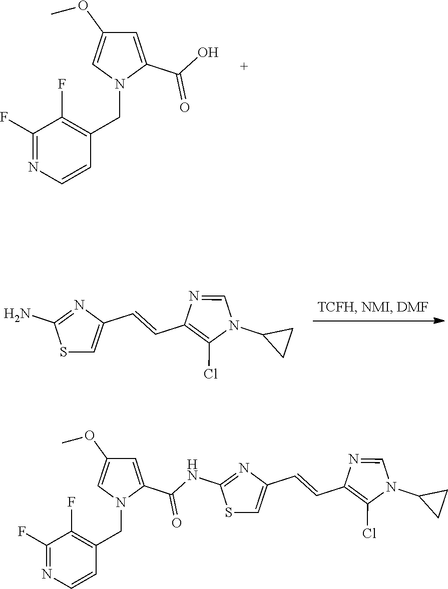 [SMILES: COc1cc(C(=O)O)n(Cc2ccnc(F)c2F)c1 ; Nc1nc(/C=C/c2ncn(C3CC3)c2Cl)cs1 ; COc1cc(C(=O)Nc2nc(/C=C/c3ncn(C4CC4)c3Cl)cs2)n(Cc2ccnc(F)c2F)c1]

Into a vial were placed 1-[(2,3-difluoropyridin-4-yl)methyl]-4-methoxypyrrole-2-carboxylic acid and 4-[(E)-2-(5-chloro-1-cyclopropylimidazol-4-yl)ethenyl]-1,3-thiazol-2-amine (1.20 equiv), NMI (3.5 equiv) in DMF (5 mL), TCFH (1.20 equiv) was added. The vial was evacuated and backflushed with nitrogen. The vial was capped and placed in a 40° C. bath. The reaction mixture was stirred at 40° C. for 12 h. The reaction mixture was cooled to room temperature. The reaction mixture was quenched with H₂O (20 mL). The resulting mixture was extracted with EtOAc (3×20 ml) and washed with brine (3×20 mL). The organic layer was dried over Na₂SO₄, filtered and concentrated in vacuo. The reaction mixture was purified via RP column to yield the desired product A20. LC-MS (ES⁺): [M+1]⁺=517.

The following compounds were prepared in a similar manner.

| | Observed | |
|---|---|---|
| | molecular | |
| | ion | Compound name |
| A18 | 485 | (E)-N-(4-(2-(5-chloro-1-cyclopropyl-1H-imidazol-4-yl)vinyl)thiazol-2-yl)-1-((2- |
| | | fluoropyridin-4-yl)methyl)-4-hydroxy-1H-pyrrole-2-carboxamide |
| A19 | 517 | (E)-N-(4-(2-(5-chloro-1-cyclopropyl-1H-imidazol-4-yl)vinyl)thiazol-2-yl)-4- |
| | | (fluoromethoxy)-1-((2-fluoropyridin-4-yl)methyl)-1H-pyrrole-2-carboxamide |
| A22 | 520 | (E)-N-(4-(2-(5-chloro-1-cyclopropyl-1H-imidazol-4-yl)vinyl)thiazol-2-yl)-1-((2,3- |
| | | difluoropyridin-4-yl)methyl)-4-(methoxy-d3)-1H-pyrrole-2-carboxamide |
| B27 | 445 | (E)-N-(4-(2-(benzofuran-5-yl)vinyl)thiazol-2-yl)-1-((2-fluoropyridin-4-yl)methyl)-1H- |
| | | pyrrole-2-carboxamide |
| A23 | 549 | (E)-N-(4-(2-(5-chloro-1-cyclopropyl-1H-imidazol-4-yl)vinyl)thiazol-2-yl)-4- |
| | | (difluoromethoxy)-1-((2-fluoro-3-methylpyridin-4-yl)methyl)-1H-pyrrole-2-carboxamide |
| A24 | 549 | (E)-N-(4-(2-(5-chloro-1-cyclopropyl-1H-imidazol-4-yl)vinyl)thiazol-2-yl)-4- |
| | | (difluoromethoxy)-1-((2-fluoro-5-methylpyridin-4-yl)methyl)-1H-pyrrole-2-carboxamide |
| B29 | 467 | (E)-N-(4-(2-(4,4-difluorocyclohexyl)vinyl)thiazol-2-yl)-1-((2-fluoropyridin-4-yl)methyl)-1H- |
| | | pyrrole-2-carboxamide |
| B28 | 423 | (E)-1-((2-fluoropyridin-4-yl)methyl)-N-(4-(3-fluorostyryl)thiazol-2-yl)-1H-pyrrole-2- |
| | | carboxamide |
| A26 | 563 | (E)-N-(4-(2-(5-chloro-1-cyclopropyl-1H-imidazol-4-yl)vinyl)thiazol-2-yl)-4- |
| | | (difluoromethoxy)-1-((2-fluoro-3,5-dimethylpyridin-4-yl)methyl)-1H-pyrrole-2- |
| | | carboxamide |
| A27 | 526 | (E)-N-(4-(2-(5-cyano-1-cyclopropyl-1H-imidazol-4-yl)vinyl)thiazol-2-yl)-4- |
| | | (difluoromethoxy)-1-((2-fluoropyridin-4-yl)methyl)-1H-pyrrole-2-carboxamide |
| A28 | 494 | (E)-4-chloro-N-(4-(2-(5-cyano-1-cyclopropyl-1H-imidazol-4-yl)vinyl)thiazol-2-yl)-1-((2- |
| | | fluoropyridin-4-yl)methyl)-1H-pyrrole-2-carboxamide |
| A29 | 519 | (E)-N-(4-(2-(5-chloro-1-cyclopropyl-1H-imidazol-4-yl)vinyl)thiazol-2-yl)-4- |
| | | (difluoromethyl)-1-((2-fluoropyridin-4-yl)methyl)-1H-pyrrole-2-carboxamide |
| A30 | 537 | (E)-N-(4-(2-(5-chloro-1-cyclopropyl-1H-imidazol-4-yl)vinyl)thiazol-2-yl)-1-((2- |
| | | fluoropyridin-4-yl)methyl)-4-(trifluoromethyl)-1H-pyrrole-2-carboxamide |
| A31 | 569 | (E)-N-(4-(2-(5-chloro-1-cyclopropyl-1H-imidazol-4-yl)vinyl)thiazol-2-yl)-1-((2- |
| | | fluoropyridin-4-yl)methyl)-4-((trifluoromethyl)thio)-1H-pyrrole-2-carboxamide |
| A32 | 551 | (E)-N-(4-(2-(5-chloro-1-cyclopropyl-1H-imidazol-4-yl)vinyl)thiazol-2-yl)-4- |
| | | ((difluoromethyl)thio)-1-((2-fluoropyridin-4-yl)methyl)-1H-pyrrole-2-carboxamide |

### Route 5:

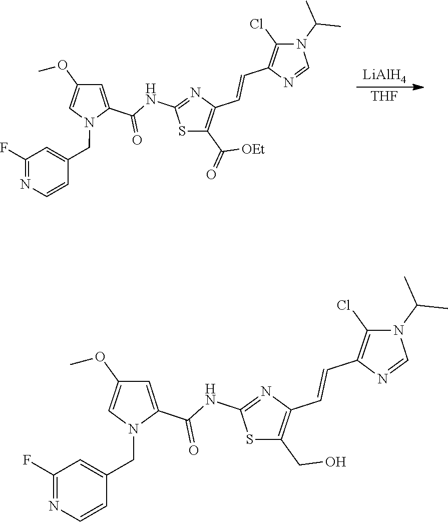 [SMILES: CCOC(=O)c1sc(NC(=O)c2cc(OC)cn2Cc2ccnc(F)c2)nc1/C=C/c1ncn(C(C)C)c1Cl ; "LiAlH4" ; COc1cc(C(=O)Nc2nc(/C=C/c3ncn(C(C)C)c3Cl)c(CO)s2)n(Cc2ccnc(F)c2)c1 ; "THF"]

Into a vial were placed ethyl 4-[(E)-2-(5-chloro-1-isopropylimidazol-4-yl)ethenyl]-2-{1-[(2-fluoropyridin-4-yl)methyl]-4-methoxypyrrole-2-amido}-1,3-thiazole-5-carboxylate in THE (10 mL), LiAlH₄ (2.00 equiv) was added in portions at −20° C. under nitrogen atmosphere. The vial was evacuated and backflushed with nitrogen. The vial was capped and placed in a −20° C. bath. The reaction mixture was stirred at −20° C. for 2 h. The reaction was quenched by the addition of Water (20 mL) at −20° C. The aqueous layer was extracted with EtOAc (2×25 mL). The organic layer was dried over Na₂SO₄, filtered and concentrated in vacuo. The resulting crude material was purified via Prep-HPLC to yield the desired product of A21. LC-MS (ES+): [M+H]⁺=531.

### Biological Assays

### Dox-Induced PD1-ss-Gluc Assay

Flp-In 293 T-REx™ cells were transfected with pcDNA™5/FRT/TO plasmid inserted with cDNA encoding Gaussia Luciferase fused to the 3′ end of cDNA encoding PD1 signal sequence plus 10 amino acids (N-MQIPQAPWPWWAVLQLGWRPGWFLDSPDR-C) (SEQ ID NO: 1). Transfected cells were selected for resistance to the selectable markers Hygromycin and Blasticidin to create a stable cell line that contained the PD1-ss+10aa/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO₂. 24 hours later, coelenterazine substrate was added to each well and luciferase signal was quantified using Tecan Infinite M1000 Pro for potency determination.

Results for select compounds provided herein are shown in the Tables below. For chemical structures that include one or more stereoisomers, but are illustrated without indicating stereochemistry, the assay data refers to a mixture of stereoisomers.

### Dox Induced TNFα-FL-Gluc Assay

Flp-In 293 T-REx™ cells were transfected with pcDNA™5/FRT/TO plasmid inserted with cDNA encoding Gaussia Luciferase fused to the 3′ end of cDNA encoding full length TNFα (amino acids 1-233). Transfected cells were selected for resistance to the selectable markers Hygromycin and Blasticidin to create a stable cell line that contained the TNFα-FL/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO₂. 24 hours later, coelenterazine substrate was added to each well and luciferase signal was quantified using Tecan Infinite M1000 Pro for potency determination.

Results for select compounds provided herein are shown in the Tables below. For chemical structures that include one or more stereoisomers, but are illustrated without indicating stereochemistry, the assay data refers to a mixture of stereoisomers.

### Dox-Inducible Her3-ss-Gluc Assay

Flp-In 293 T-REx™ cells were transfected with pcDNA™5/FRT/TO plasmid inserted with cDNA encoding Gaussia Luciferase fused to the 3′ end of cDNA encoding HER3 signal sequence plus 4 amino acids (N-MRANDALQVLGLLFSLARGSEVG-C) (SEQ ID NO: 2). Transfected cells were selected for resistance to the selectable markers Hygromycin and Blasticidin to create a stable cell line that contained the HER3-ss+4aa/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO₂. 24 hours later, coelenterazine substrate was added to each well and luciferase signal was quantified using Tecan Infinite M1000 Pro for potency determination.

Results for select compounds provided herein are shown in the Tables below. For chemical structures that include one or more stereoisomers, but are illustrated without indicating stereochemistry, the assay data refers to a mixture of stereoisomers.

### Dox Inducible IL2-FL-Gluc Assay

Flp-In 293 T-REx™ cells were transfected with pcDNA™5/FRT/TO plasmid inserted with cDNA encoding Gaussia Luciferase fused to the 3′ end of cDNA encoding full length IL-2 (amino acids 1-153). Transfected cells were selected for resistance to the selectable markers Hygromycin and Blasticidin to create a stable cell line that contained the IL-2-FL/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO₂. 24 hours later, coelenterazine substrate was added to each well and luciferase signal was quantified using Tecan Infinite M1000 Pro for potency determination.

Results for select compounds provided herein are shown in the Tables below. For chemical structures that include one or more stereoisomers, but are illustrated without indicating stereochemistry, the assay data refers to a mixture of stereoisomers.

### Dox-induced IL-6R-ss-Gluc Assay

Flp-In 293 T-REx™ cells were transfected with pcDNATM5/FRT/TO plasmid inserted with cDNA encoding Gaussia Luciferase fused to the 3′ end of cDNA encoding IL6R signal sequence plus 10 amino acids (N-MLAVGCALLAALLAAPGAALAPRRCPAQE-C). Transfected cells were selected for resistance to the selectable markers Hygromycin and Blasticidin to create a stable cell line that contained the IL6R-ss+10aa/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO2. 24 hours later, coelenterazine substrate was added to each well and luciferase signal was quantified using a multi-mode plate reader for potency determination.

Results for select compounds provided herein are shown in the Tables below. For chemical structures that include one or more stereoisomers, but are illustrated without indicating stereochemistry, the assay data refers to a mixture of stereoisomers.

### Dox-Induced CD47-ss-Gluc Assay

Flp-In 293 T-REx™ cells were transfected with pcDNATM5/FRT/TO plasmid inserted with cDNA encoding Gaussia Luciferase fused to the 3′ end of cDNA encoding PD1 signal sequence plus 10 amino acids (N-MWPLVAALLLGSACCGSAQLLFNKTKSV-C). Transfected cells were selected for resistance to the selectable markers Hygromycin and Blasticidin to create a stable cell line that contained the PD1-ss+10aa/Gaussia Luciferase cDNA insert whose expression was regulated under the T-REx™ system. The day before assay, cells were trypsinized and plated in 384-well tissue culture plates. The next day, compound dilutions in DMSO/media containing doxycycline were added to the wells and incubated at 37° C., 5% CO2. 24 hours later, coelenterazine substrate was added to each well and luciferase signal was quantified using a multi-mode plate reader for potency determination.

Results for select compounds provided herein are shown in the Tables below. For chemical structures that include one or more stereoisomers, but are illustrated without indicating stereochemistry, the assay data refers to a mixture of stereoisomers.

### H929 Cell Viability Assay

The human multiple myeloma cell line NCI-H929 was cultured in Advanced RPMI 1640 media (Gibco®) supplemented with 6% fetal bovine serum, 2 mM Glutamine, and 1× Penicillin/Streptomycin. On the day of assay, cells were resuspended in RPMI 1640 media supplemented with 10% fetal bovine serum, 2 mM Glutamine, and 1× Penicillin/Streptomycin and plated in 384-well tissue culture plates and treated with compound dilutions in DMSO/media. Plates were incubated at 37° C., 5% CO₂ for 48 hours. After 48 hours, Celltiter-Glo® (Promega) was added to each well and luciferase signal was quantified using Tecan Infinite M1000 Pro for cell viability determination.

Results for select compounds provided herein are shown in the Tables below. For chemical structures that include one or more stereoisomers, but are illustrated without indicating stereochemistry, the assay data refers to a mixture of stereoisomers.

### U266 Cell Viability Assay

The human multiple myeloma cell line U266B1 was cultured in RPMI 1640 media supplemented with 10% fetal bovine serum, 2 mM Glutamine, and 1× Penicillin/Streptomycin. Cells were plated in 384-well tissue culture plates and treated with compound dilutions in DMSO/media. Plates were incubated at 37° C., 5% CO₂ for 48 hours. After 48 hours, Celltiter-Glo® (Promega) was added to each well and luciferase signal was quantified using Tecan Infinite M1000 Pro for cell viability determination.

Results for select compounds provided herein are shown in the Tables below. For chemical structures that include one or more stereoisomers, but are illustrated without indicating stereochemistry, the assay data refers to a mixture of stereoisomers.

### Liver Microsome Stability Assays

Stability of a compound was assessed in the presence of liver microsomes from various sources—mouse, rat, monkey and human liver microsomes. 1.0 uM compound, 0.4% DMSO in 0.1 M Potassium Phosphate with 1.0 mg/mL liver microsomes, were incubated at 37° C. with or without 1 mM NADPH. The samples were quenched at 0, and 30 minutes.

TABLE 1

| | | | | 24 hr Dox | 24hr Dox | |
|---|---|---|---|---|---|---|
| | 24 hr Dox | 24 hr Dox | | Inducible | Inducible | 24hr Dox |
| | Inducible | Inducible | 24 hr Dox | CD47(ss + | IL6R(ss + | Inducible |
| | PD1ssGluc | IL2FLGluc | Inducible | 10)Gluc | 10)Gluc | IL6R(ss + |
| | 293FRT/TO: | 293FRT/TO: | IL2FLGluc | 293FRT/TO: | 293FRT/TO: | 10)Gluc |
| Compound | Mean IC50 | Mean IC50 | 293FRT/TO: | Mean IC50 | Mean IC50 | 293FRT/TO: |
| ID | (nM) | (nM) | IL2/PD1 | (nM) | (nM) | IL6R/PD1 |
| A1 | 30 | 2023 | 66 | 1864 | 1890 | 62 |
| A2 | 24 | 4169 | 170 | 3805 | 3268 | 133 |
| A3 | 1759 | 12220 | 7 | 15206 | 4678 | 3 |
| A4 | 604 | 12683 | 21 | 6228 | 6766 | 11 |
| A5 | 436 | I.A. | >57 | I.A. | I.A. | >57 |
| A6 | 85 | 2422 | 29 | 2163 | 2334 | 28 |
| A7 | 14 | 2701 | 200 | 2101 | 2432 | 176 |
| A8 | 129 | 1818 | 14 | 1740 | 1955 | 15 |
| A9 | I.A. | I.A. | N/A | I.A. | I.A. | N/A |
| A10 | 11 | 1072 | 97 | 933 | 1055 | 96 |
| A11 | 11 | 1178 | 110 | 1455 | 1196 | 112 |
| A13 | 5 | 2308 | 440 | 2028 | 2560 | 489 |
| A14 | 7 | 3442 | 520 | 3312 | 2112 | 318 |
| A15 | 6 | 2937 | 490 | 3229 | 2073 | 345 |
| A16 | 6 | 2913 | 460 | 2803 | 2151 | 339 |
| A17 | 9 | 3016 | 340 | 2741 | 2100 | 240 |
| A18 | 295 | 6998 | 24 | 6411 | 5530 | 19 |
| A19 | 10 | 3663 | 360 | 4353 | 2907 | 287 |
| A20 | 31 | I.A. | >810 | 5011 | 3763 | 123 |
| A21 | 238 | I.A. | >110 | 5866 | I.A. | >105 |
| A22 | 34 | I.A. | >740 | 5534 | I.A. | >741 |
| A23 | 37 | I.A. | >540 | >25000 | I.A. | >680 |
| A24 | 8 | 11542 | 1100.0 | 5527 | 9973 | 1283 |
| A26 | 877 | I.A. | >22 | I.A. | I.A. | 28 |
| A27 | 9 | 3088 | 340.0 | 2546 | 2111 | 235 |
| A28 | 22 | 1505 | 69.0 | 1403 | 1489 | 68 |
| A29 | 48 | I.A. | >650 | I.A. | I.A. | >517 |
| A30 | 7 | 2128 | 290.0 | 2385 | 2163 | 292 |
| A31 | 4 | I.A. | >6300 | 2212 | 1264 | 317 |
| A32 | 10 | I.A | >2400 | 3063 | 1944 | 187 |
| B11 | 18695 | 14137 | 0.8 | I.A. | I.A. | >1.3 |
| B12 | 17110 | 7131 | 0.4 | 15568 | I.A. | >1.5 |
| B13 | 50 | I.A. | >500 | I.A. | I.A. | >500 |
| B14 | 1103 | I.A. | >23 | I.A. | I.A. | >23 |
| B15 | 11885 | I.A. | >2.1 | I.A. | I.A. | >2 |
| B16 | 8114 | 19523 | 2.4 | 7071 | 8771 | 1 |
| B17 | 5098 | 8239 | 1.6 | I.A. | 23753 | 5 |
| B18 | 11297 | I.A. | >2.2 | I.A. | 9912 | 1 |
| B19 | 318 | I.A. | >79 | I.A. | I.A. | >79 |
| B20 | 9 | 1533 | 170.0 | 1523 | 1320 | 146 |
| B21 | 65 | I.A. | >380 | I.A. | I.A. | >385 |
| B22 | I.A. | I.A. | N/A | I.A. | I.A. | N/A |
| B27 | 144 | 1044 | 5.2 | 1032 | 892 | 6 |
| B28 | 172 | 1090 | 6.1 | 1005 | 1201 | 7 |
| B29 | 112 | 1432 | 12.0 | 838 | 922 | 8 |

I.A. indicates IC50 >25000 nM

TABLE 2

| | | | Liver Microsome | Liver Microsome | Liver Microsome | Liver Microsome |
|---|---|---|---|---|---|---|
| | 48 hr H929 | 48 hr U266 | Stability (Multiple | Stability (Multiple | Stability (Multiple | Stability (Multiple |
| | Viability | Viability | Species): Mouse - | Species): Rat - | Species): Monkey - | Species): Human - |
| | Celltiter-Glo: | Celltiter-Glo: | % Remaining after | % Remaining after | % Remaining after | % Remaining after |
| | Mean EC50 | Mean EC50 | 30 min w/ NADPH | 30 min w/ NADPH | 30 min w/ NADPH | 30 min w/ NADPH |
| | (nM) | (nM) | (%) | (%) | (%) | (%) |
| A1 | I.A. | I.A. | 71 | 82 | 31 | 11 |
| A2 | I.A. | I.A. | 68 | 72 | 35 | 15 |
| A3 | 4548 | >20171 | 61 | 21 | 4 | 6 |
| A4 | >23262 | I.A. | 80 | 73 | 4 | 3 |
| A5 | 10089 | 16797 | 73 | 42 | 2 | 4 |
| A6 | I.A | I.A. | 96 | 76 | 30 | 48 |
| A7 | 19501 | I.A. | 82 | 84 | 55 | 64 |
| A8 | 7439 | 12809 | 86 | 47 | 68 | 72 |
| A9 | I.A. | I.A. | 64 | 66 | 9 | 11 |
| A10 | 5448 | I.A. | 87 | 77 | 38 | 27 |
| A11 | 11903 | I.A. | 87 | 17 | 6 | 14 |
| A12 | 10228 | I.A. | 78 | 17 | 5 | 8 |
| A13 | I.A. | I.A. | 16 | 62 | 9 | 48 |
| A14 | I.A. | I.A. | 76 | 20 | 4 | 29 |
| A15 | I.A | I.A. | 100 | 55 | 57 | 73 |
| A16 | I.A. | I.A. | 81 | 56 | 58 | 83 |
| A17 | I.A | I.A. | 72 | 2 | 17 | 26 |
| A18 | 24680 | I.A. | 33 | 10 | 2 | 1 |
| A19 | I.A. | I.A. | 65 | 23 | 51 | 4 |
| A20 | I.A. | I.A. | 74 | 41 | 28 | 52 |
| A21 | I.A. | I.A. | 51 | 59 | <1 | 1 |
| A22 | I.A. | I.A. | 69 | 33 | 9 | 16 |
| A23 | I.A. | I.A. | 55 | 31 | 8 | 20 |
| A24 | I.A. | I.A. | 77 | 30 | 12 | 20 |
| A25 | 11900 | 10492 | 72 | 84 | 27 | 56 |
| A26 | I.A. | I.A. | 70 | 40 | 16 | 29 |
| A27 | 17800 | I.A. | 70 | 2 | 47 | 78 |
| A28 | I.A. | 15533 | 67 | 2 | 9 | 22 |
| A29 | I.A. | I.A. | 21 | 1 | 43 | 51 |
| A30 | I.A. | I.A. | 44 | <1 | 70 | 63 |
| A31 | 22683 | I.A. | 82 | 65 | 25 | 10 |
| A32 | I.A. | I.A. | 27 | 5 | 2 | 1 |
| B1 | I.A. | I.A. | 29 | 3 | 2 | 10 |
| B2 | I.A. | I.A. | 6 | 8 | <1 | <1 |
| B3 | I.A. | I.A. | 53 | 19 | 1 | <1 |
| B4 | 8746 | 19445 | 7 | 1 | <1 | <1 |
| B5 | I.A. | I.A. | 20 | 20 | <1 | 4 |
| B6 | I.A. | I.A. | 63 | 41 | 24 | 42 |
| B7 | 8088 | I.A. | 23 | 13 | 6 | 4 |
| B8 | I.A. | I.A. | 11 | 1 | 4 | 22 |
| B9 | 6260 | 19986 | 6 | 4 | 1 | 6 |
| B10 | 5807 | I.A. | 49 | 5 | 11 | 38 |
| B11 | 24698 | I.A. | 54 | 32 | 3 | 1 |
| B12 | I.A. | I.A. | 57 | 12 | <1 | <1 |
| B13 | I.A. | I.A. | 4 | <1 | <1 | <1 |
| B14 | I.A. | I.A. | 0.2 | <1 | <1 | <1 |
| B15 | I.A. | I.A. | 2 | 1 | <1 | <1 |
| B16 | 19261 | 21338 | 79 | 47 | 2 | 1 |
| B17 | 5107 | 8612 | 4 | 17 | <1 | <1 |
| B18 | I.A. | I.A. | 45 | 6 | <1 | <1 |
| B19 | I.A. | I.A. | 22 | 1 | 6 | 7 |
| B20 | 8361 | 15673 | 5 | 33 | <1 | <1 |
| B21 | 17505 | 24203 | 11 | 20 | <1 | <1 |
| B22 | I.A. | I.A. | 41 | 1 | <1 | 9 |
| B23 | I.A. | I.A. | 16 | 19 | 1 | <1 |
| B24 | 13553 | 15326 | 3 | 1 | <1 | 1 |
| B25 | 13679 | 17294 | 0.05 | <1 | <1 | <1 |
| B26 | 7133 | 19722 | 31 | 10 | <1 | 59 |
| B27 | I.A. | I.A. | 40 | 3 | 8 | 27 |
| B28 | 15000 | I.A. | 72 | 39 | 22 | 70 |

I.A. indicates IC50 >25000 nM

TABLE 3

| | | | | 24 hr Dox | | |
|---|---|---|---|---|---|---|
| | 24 hr Dox | 24 hr Dox | | Inducible | 24 hr Dox | |
| | Inducible | Inducible | 24 hr Dox | Her3(ss + | Inducible | 24 hr Dox |
| | PD1ssGluc | IL2FLGluc | Inducible | 4)Gluc | TNFaFLGluc | Inducible |
| | 293FRT/TO: | 293FRT/TO: | IL2FLGluc | 293FRT/TO: | 293FRT/TO: | TNFaFLGluc |
| Compound | Mean IC50 | Mean IC50 | 293FRT/TO: | Mean IC50 | Mean IC50 | 293FRT/TO: |
| ID | (nM) | (nM) | IL2/PD1 | (nM) | (nM) | TNF/PD1 |
| A12 | 11 | 1534 | 150 | 1147 | 8536 | 807 |
| B1 | 8932 | I.A. | >2.8 | I.A. | I.A. | >2 |
| B2 | 4475 | 6636 | 2 | 6402 | I.A. | >5 |
| B3 | 644 | I.A. | >39 | I.A. | I.A. | >38 |
| B4 | 13 | 1647 | 130 | 816 | 7039 | 544 |
| B5 | 17 | 1198 | 70 | 553 | 3937 | 230 |
| B6 | 31 | 265 | 9 | 137 | 445 | 14 |
| B7 | 30 | 499 | 17 | 184 | 3982 | 134 |
| B8 | 24 | 1677 | 69 | 1180 | 11167 | 462 |
| B9 | 161 | 748 | 5 | 298 | 3969 | 25 |
| B10 | 41 | 249 | 6 | 86 | 1007 | 25 |
| B23 | 59 | I.A. | >420 | 3392 | I.A. | >422 |
| B24 | 540 | I.A. | >46 | I.A. | I.A. | >46 |
| B25 | 404 | I.A. | >62 | I.A. | I.A. | >61 |
| B26 | 117 | 737 | 6 | 332 | 3824 | 33 |

### EMBODIMENTS

1. A compound, or pharmaceutically acceptable salt thereof, having a structure of formula (I):

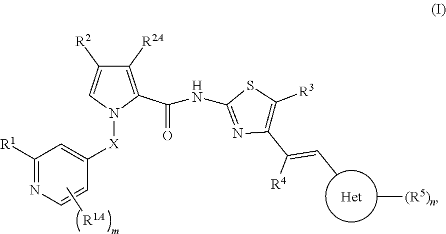

R¹ is H or F;
m is 0, 1, or 2;
each R^{1A} is independently F or C₁₋₃alkyl;
X is CH₂ or CD₂;
one of R² and R^{2A} is halo, CN, OH, C₁₋₃haloalkyl, C₁₋₃alkoxy, C₁₋₃haloalkoxy, —S—C₁₋₃alkyl, or —S—C₁₋₃haloalkyl, and the other is H;
R³ is H, halo, CH₂OH, or OCH₃;
R⁴ is H, D, CH₃, or halo;
each R⁵ is independently selected from D, C₁₋₃alkyl, cyclopropyl, cyano-substituted cyclopropyl, O-cyclopropyl, C₁₋₃haloalkyl, C₁₋₃haloalkoxy, CH₂OH, halo, oxo, CO₂R^{N}, C₀₋₂alkylene-C(O)N(R^{N})₂, N(R^{N})₂, N(R^{N})C(O)R^{N}, and CN;
Het is imidazolyl, oxazolyl, triazolyl, pyridiyl, cyclohexyl, tetrahydrofuranyl, 5, 6, 7, 8-tetrahydroimidazo[1,2,a]pyridyl, or 5, 6, 7, 8-tetrahydroimidazo[1,2,a]pyrimidinyl;
n is 0, 1 or 2, with the proviso that when Het is imidazolyl, then (a) n is 1 and R⁵ is not cyclopropyl, or (b) n is 2 and either (i) at least one R⁵ is halo, CN, C₁₋₃haloalkyl, or C₁₋₃haloalkoxy, or (ii) each R⁵ is C₁₋₃alkyl; and
each R^{N} is independently H or C₁₋₆alkyl, or two R^{N} on the same nitrogen atom, together with the nitrogen to which they are attached, form a 4-7 membered ring having 0-2 additional ring heteroatoms independently selected from N, O, and S.
   2. The compound or salt of embodiment 1, wherein R¹ is F.
   3. The compound or salt of embodiment 1 or 2, wherein m is 0.
   4. The compound or salt of embodiment 1 or 2, wherein m is 1.
   5. The compound or salt of embodiment 4, wherein R^{1A} is F.
   6. The compound or salt of embodiment 1 or 2, wherein m is 2.
   7. The compound or salt of embodiment 6, wherein at least one R^{1A} is F.
   8. The compound or salt of embodiment 7, wherein each R^{1A} is F.
   9. The compound or salt of embodiment 6 or 7, wherein at least one R^{1A} is CH₃.
   10. The compound or salt of embodiment 9, wherein each R^{1A} is CH₃.
   11. The compound or salt of any one of embodiments 1 to 10, wherein R⁴ is H.
   12. The compound or salt of any one of embodiments 1 to 10, wherein R⁴ is CH₃.
   13. The compound or salt of any one of embodiments 1 to 10, wherein R⁴ is halo.
   14. The compound or salt of any one of embodiments 1 to 13, wherein Het is triazolyl, cyclohexyl, or tetrahydrofuranyl.
   15. The compound or salt of any one of embodiments 1 to 13, wherein Het is oxazolyl or pyridyl.
   16. The compound or salt of any one of embodiments 1 to 13, wherein Het is 5, 6, 7, 8-tetrahydroimidazo[1,2,a]pyridyl or 5, 6, 7, 8-tetrahydroimidazo[1,2,a]pyrimidinyl.
   17. The compound or salt of any one of embodiments 1 to 13, wherein Het is imidazoyl.
   18. The compound or salt of any one of embodiments 1 to 16, wherein n is 0.
   19. The compound or salt of any one of embodiments 1 to 17, wherein n is 1.
   20. The compound or salt of any one of embodiments 1 to 17, wherein n is 2.
   21. The compound or salt of embodiment 19 or 20, wherein at least one R⁵ is halo.
   22. The compound or salt of any one of embodiments 19 to 21, wherein at least one R⁵ is C₁₋₃alkyl, cyano, cyclopropyl, cyano-substituted cyclopropyl, O-cyclopropyl, C₁₋₃haloalkyl, C₁₋₃haloalkoxy, or CH₂OH.
   23. The compound or salt of any one of embodiments 1 to 13, wherein

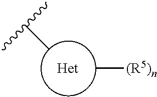

is selected from the group consisting of

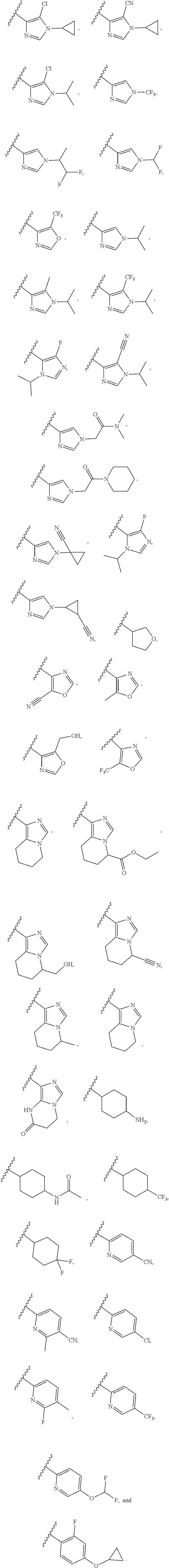

24. The compound or salt of any one of embodiments 1 to 23, wherein X is CH₂.
25. The compound or salt of any one of embodiments 1 to 23, wherein X is CD₂.
26. The compound or salt of any one of embodiments 1 to 25, wherein R² is halo, CN, C₁₋₃alkoxy, or C₁₋₃ haloalkoxy.
27. The compound or salt of embodiment 26, wherein R² is halo, CN, C₁₋₃alkoxy, or C₁₋₃haloalkoxy.
28. The compound or salt of embodiment 27, wherein R² is F, Cl, CN, OCH₃, OCD₃, or OCHF₂.
29. The compound or salt of any one of embodiments 1 to 25, wherein R² is OH, CHF₂, CF₃, —SCF₃, or —SCHF₂.
30. The compound or salt of any one of embodiments 1 to 25, wherein R^{2A} is halo, CN, C₁₋₃alkoxy, or C₁₋₃ haloalkoxy.
31. The compound or salt of embodiment 30, wherein R^{2A} is halo, CN, C₁₋₃alkoxy, or C₁₋₃haloalkoxy.
32. The compound or salt of embodiment 31, wherein R^{2A} is F, Cl, CN, OCH₃, OCD₃, or OCHF₂.
33. The compound or salt of any one of embodiments 1 to 25, wherein R^{2A} is OH, CHF₂, CF₃, —SCF₃, or —SCHF₂.
34. The compound or salt of any one of embodiments 1 to 33, wherein R³ is H, halo, or OCH₃.
35. The compound or salt of claim 34, wherein R³ is H.
36. The compound or salt of embodiment 34, wherein R³ is halo or OCH₃.
37. The compound or salt of any one of embodiments 1 to 33, wherein R³ is CH₂OH.
38. A compound, or pharmaceutically acceptable salt thereof, as recited in Table A or Table B.
39. The compound or salt of embodiment 38, selected from the group consisting of A1-A17 and B1-B26.
40. The compound or salt of embodiment 39, selected from the group consisting of A1, A2, A6, A7, A10, A11, A12, A13, A14, A15, A16, A17, B2, and B20.
41. A pharmaceutical composition comprising the compound or salt of any one of embodiments 1 to 40 and a pharmaceutically acceptable excipient.
42. A method of inhibiting protein secretion in a cell comprising contacting the cell with the compound or salt of any one of embodiments 1 to 40 in an amount effective to inhibit secretion.
43. The method of embodiment 42, wherein the protein is a checkpoint protein.
44. The method of embodiment 42, wherein the protein is a cell-surface protein, endoplasmic reticulum associated protein, or secreted protein involved in regulation of anti-tumor immune response.
45. The method of embodiment 42, wherein the protein is at least one of PD-1, PD-L1, TIM-1, LAG-3, CTLA4, BTLA, OX-40, B7H1, B7H4, CD137, CD47, CD96, CD73, CD40, VISTA, TIGIT, LAIR1, CD160, 2B4, TGFRβ and combinations thereof.
46. The method of embodiment 42, wherein the protein is selected from the group consisting of HER3, TNFα, IL2, and PD-1.
47. The method of embodiment 42, wherein the protein is PD-1.
48. The method of any one of embodiments 42 to 47, wherein the contacting comprises administering the compound to a subject in need thereof.
49. A method for treating inflammation in a subject comprising administering to the subject a therapeutically effective amount of the compound or salt of any one of embodiments 1 to 40.
50. A method for treating cancer in a subject comprising administering to the subject a therapeutically effective amount of the compound or salt of any one of embodiments 1 to 40.
51. The method of embodiment 50, wherein the cancer is melanoma, multiple myeloma, prostate cancer, lung cancer, pancreatic cancer, squamous cell carcinoma, leukemia, lymphoma, a neuroendocrine tumor, bladder cancer, or colorectal cancer.
52. The method of embodiment 50, wherein the cancer is selected from the group consisting of prostate, lung, bladder, colorectal, and multiple myeloma.
53. The method of embodiment 50, wherein the cancer is non-small cell lung carcinoma, squamous cell carcinoma, leukemia, acute myelogenous leukemia, chronic myelogenous leukemia, lymphoma, NPM/ALK-transformed anaplastic large cell lymphoma, diffuse large B cell lymphoma, neuroendocrine tumors, breast cancer, mantle cell lymphoma, renal cell carcinoma, rhabdomyosarcoma, ovarian cancer, endometrial cancer, small cell carcinoma, adenocarcinoma, gastric carcinoma, hepatocellular carcinoma, pancreatic cancer, thyroid carcinoma, anaplastic large cell lymphoma, hemangioma, or head and neck cancer.
54. The method of embodiment 50, wherein the cancer is a solid tumor.
55. The method of embodiment 50, wherein the cancer is head and neck cancer, squamous cell carcinoma, gastric carcinoma, or pancreatic cancer.
56. A method for treating an autoimmune disease in a subject comprising administering to the subject a therapeutically effective amount of the compound or salt of any one of embodiments 1 to 40.
57. The method of embodiment 56, wherein the autoimmune disease is psoriasis, dermatitis, systemic scleroderma, sclerosis, Crohn's disease, ulcerative colitis; respiratory distress syndrome, meningitis; encephalitis; uveitis; colitis; glomerulonephritis; eczema, asthma, chronic inflammation; atherosclerosis; leukocyte adhesion deficiency; rheumatoid arthritis; systemic lupus erythematosus (SLE); diabetes mellitus; multiple sclerosis; Reynaud's syndrome; autoimmune thyroiditis; allergic encephalomyelitis; Sjorgen's syndrome; juvenile onset diabetes; tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.
58. A method for the treatment of an immune-related disease in a subject comprising administering to the subject a therapeutically effective amount of the compound or salt of any one of embodiments 1 to 40.
59. The method of embodiment 58, wherein the immune-related disease is rheumatoid arthritis, lupus, inflammatory bowel disease, multiple sclerosis, or Crohn's disease.
60. A method for treating neurodegenerative disease in a subject comprising administering to the subject a therapeutically effective amount of the compound or salt of any one of embodiments 1 to 40.
61. The method of embodiment 60, wherein the neurodegenerative disease is multiple sclerosis.
62. A method for treating an inflammatory disease in a subject comprising administering to the subject a therapeutically effective amount of the compound or salt of any one of embodiments 1 to 40.
63. The method of embodiment 62, wherein the inflammatory disease is bronchitis, conjunctivitis, myocarditis, pancreatitis, chronic cholecstitis, bronchiectasis, aortic valve stenosis, restenosis, psoriasis or arthritis.

What is claimed is:

1. A compound, or pharmaceutically acceptable salt thereof, having a structure of formula (I): 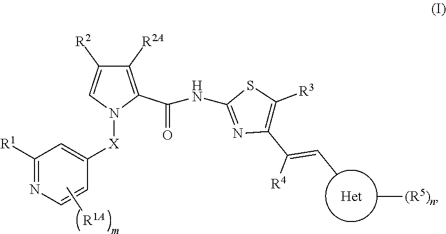
R¹ is H or F;
m is 0, 1, or 2;
each R^{1A} is independently F or C₁₋₃alkyl;
X is CH₂ or CD₂;
one of R² and R^{2A} is halo, CN, OH, C₁₋₃haloalkyl, C₁₋₃alkoxy, C₁₋₃haloalkoxy, —S—C₁₋₃alkyl, or —S—C₁₋₃haloalkyl, and the other is H;
R³ is H, halo, CH₂OH, or OCH₃;
R⁴ is H, D, CH₃, or halo;
each R⁵ is independently selected from D, C₁₋₃alkyl, cyclopropyl, cyano-substituted cyclopropyl, O-cyclopropyl, C₁₋₃haloalkyl, C₁₋₃haloalkoxy, CH₂OH, halo, oxo, CO₂R^{N}, C₀₋₂alkylene-C(O)N(R^{N})₂, N(R^{N})₂, N(R^{N})C(O)R^{N}, and CN;
Het is imidazolyl, oxazolyl, triazolyl, pyridiyl, cyclohexyl, tetrahydrofuranyl, 5, 6, 7, 8-tetrahydroimidazo[1,2,a]pyridyl, or 5, 6, 7, 8-tetrahydroimidazo[1,2,a]pyrimidinyl;
n is 0, 1 or 2, with the proviso that when Het is imidazolyl, then (a) n is 1 and R⁵ is not cyclopropyl, or (b) n is 2 and either (i) at least one R⁵ is halo, CN, C₁₋₃haloalkyl, or C₁₋₃haloalkoxy, or (ii) each R⁵ is C₁₋₃alkyl; and
each R^{N} is independently H or C₁₋₆alkyl, or two R^{N} on the same nitrogen atom, together with the nitrogen to which they are attached, form a 4-7 membered ring having 0-2 additional ring heteroatoms independently selected from N, O, and S.

2. The compound or salt of claim 1, wherein R¹ is F.

3. The compound or salt of claim 1 or 2, wherein m is 0.

4. The compound or salt of claim 1 or 2, wherein m is 1.

5. The compound or salt of claim 4, wherein R^{1A} is F.

6. The compound or salt of claim 1 or 2, wherein m is 2.

7. The compound or salt of claim 6, wherein at least one R^{1A} is F.

8. The compound or salt of claim 7, wherein each R^{1A} is F.

9. The compound or salt of claim 6 or 7, wherein at least one R^{1A} is CH₃.

10. The compound or salt of claim 9, wherein each R^{1A} is CH₃.

11. The compound or salt of any one of claims 1 to 10, wherein R⁴ is H.

12. The compound or salt of any one of claims 1 to 10, wherein R⁴ is CH₃.

13. The compound or salt of any one of claims 1 to 10, wherein R⁴ is halo.

14. The compound or salt of any one of claims 1 to 13, wherein Het is triazolyl, cyclohexyl, or tetrahydrofuranyl.

15. The compound or salt of any one of claims 1 to 13, wherein Het is oxazolyl or pyridyl.

16. The compound or salt of any one of claims 1 to 13, wherein Het is 5, 6, 7, 8-tetrahydroimidazo[1,2,a]pyridyl or 5, 6, 7, 8-tetrahydroimidazo[1,2,a]pyrimidinyl.

17. The compound or salt of any one of claims 1 to 13, wherein Het is imidazoyl.

18. The compound or salt of any one of claims 1 to 17, wherein n is 0.

19. The compound or salt of any one of claims 1 to 17, wherein n is 1.

20. The compound or salt of any one of claims 1 to 17, wherein n is 2.

21. The compound or salt of claim 19 or 20, wherein at least one R⁵ is halo.

22. The compound or salt of any one of claims 19 to 21, wherein at least one R⁵ is C₁₋₃alkyl, cyano, cyclopropyl, cyano-substituted cyclopropyl, O-cyclopropyl, C₁₋₃haloalkyl, C₁₋₃haloalkoxy, or CH₂OH.

23. The compound or salt of any one of claims 1 to 13, wherein 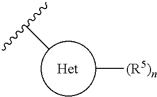
is selected from the group consisting of

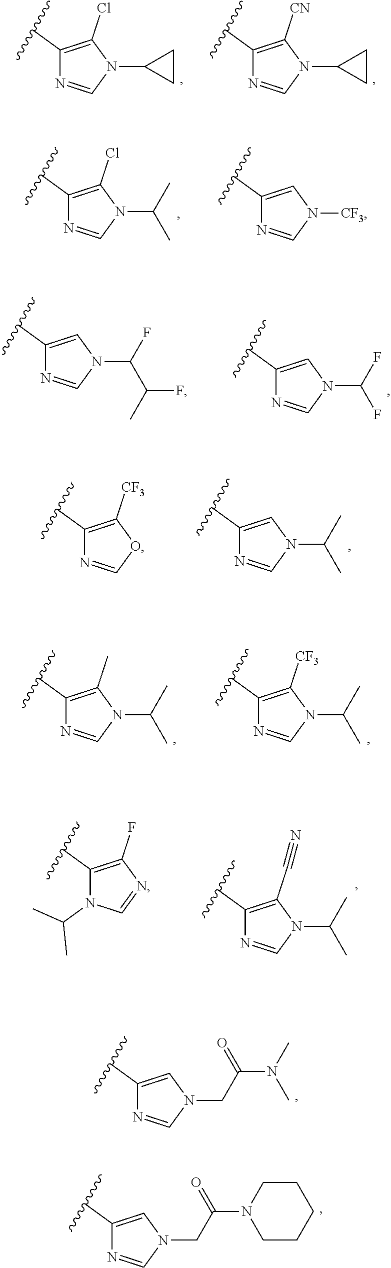

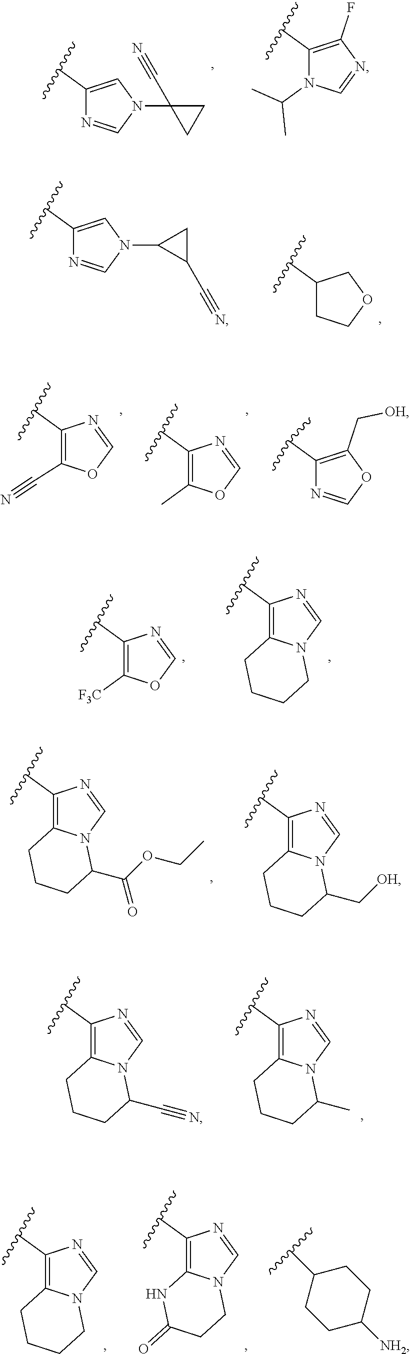

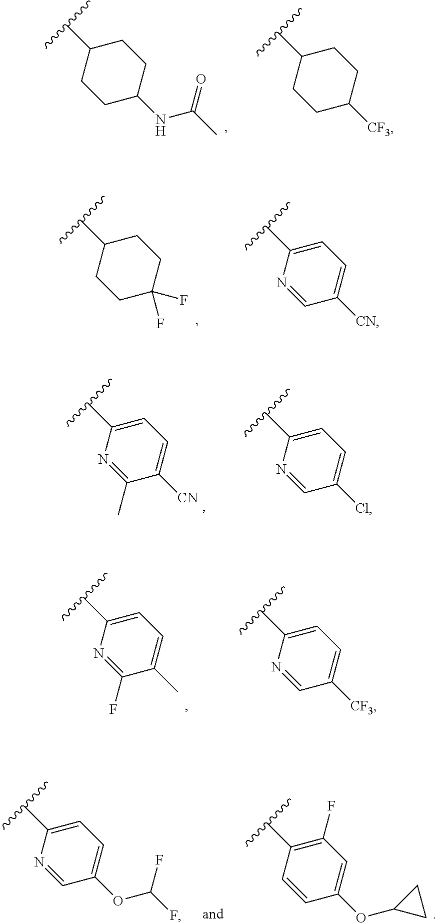

24. The compound or salt of any one of claims 1 to 23, wherein X is CH₂.

25. The compound or salt of any one of claims 1 to 23, wherein X is CD₂.

26. The compound or salt of any one of claims 1 to 25, wherein R² is halo, CN, C₁₋₃alkoxy, or C₁₋₃haloalkoxy.

27. The compound or salt of claim 26, wherein R² is halo, CN, C₁₋₃alkoxy, or C₁₋₃haloalkoxy.

28. The compound or salt of claim 27, wherein R² is F, Cl, CN, OCH₃, OCD₃, or OCHF₂.

29. The compound or salt of any one of claims 1 to 25, wherein R² is OH, CHF₂, CF₃, —SCF₃, or SCHF₂.

30. The compound or salt of any one of claims 1 to 25, R^{2A} is halo, CN, C₁₃alkoxy, or C₁₃haloalkoxy.

31. The compound or salt of claim 30, wherein R^{2A} is halo, CN, C₁₋₃alkoxy, or C₁₋₃haloalkoxy.

32. The compound or salt of claim 31, wherein R^{2A} is F, Cl, CN, OCH₃, OCD₃, or OCHF₂.

33. The compound or salt of any one of claims 1 to 25, wherein R^{2A} is OH, CHF₂, CF₃, —SCF₃, or SCHF₂.

34. The compound or salt of any one of claims 1 to 33, wherein R³ is H, halo, or OCH₃.

35. The compound or salt of claim 34, wherein R³ is H.

36. The compound or salt of claim 34, wherein R³ is halo or OCH₃.

37. The compound or salt of any one of claims 1 to 33, wherein R³ is CH₂OH.

38. A compound, or pharmaceutically acceptable salt thereof, as recited in Table A or Table B.

39. The compound or salt of claim 38, selected from the group consisting of A1-A17 and B1-B26.

40. The compound or salt of claim 39, selected from the group consisting of A1, A2, A6, A7, A10, A11, A12, A13, A14, A15, A16, A17, B2, and B20.

41. A pharmaceutical composition comprising the compound or salt of any one of claims 1 to 40 and a pharmaceutically acceptable excipient.

42. A method of inhibiting protein secretion in a cell comprising contacting the cell with the compound or salt of any one of claims 1 to 40 in an amount effective to inhibit secretion.

43. The method of claim 42, wherein the protein is a checkpoint protein.

44. The method of claim 42, wherein the protein is a cell-surface protein, endoplasmic reticulum associated protein, or secreted protein involved in regulation of anti-tumor immune response.

45. The method of claim 42, wherein the protein is at least one of PD-1, PD-L1, TIM-1, LAG-3, CTLA4, BTLA, OX-40, B7H1, B7H4, CD137, CD47, CD96, CD73, CD40, VISTA, TIGIT, LAIR1, CD160, 2B4, TGFRβ and combinations thereof.

46. The method of claim 42, wherein the protein is selected from the group consisting of HER3, TNFα, IL2, and PD-1.

47. The method of claim 42, wherein the protein is PD-1.

48. The method of any one of claims 42 to 47, wherein the contacting comprises administering the compound to a subject in need thereof.

49. A method for treating inflammation in a subject comprising administering to the subject a therapeutically effective amount of the compound or salt of any one of claims 1 to 40.

50. A method for treating cancer in a subject comprising administering to the subject a therapeutically effective amount of the compound or salt of any one of claims 1 to 40.

51. The method of claim 50, wherein the cancer is melanoma, multiple myeloma, prostate cancer, lung cancer, pancreatic cancer, squamous cell carcinoma, leukemia, lymphoma, a neuroendocrine tumor, bladder cancer, or colorectal cancer.

52. The method of claim 50, wherein the cancer is selected from the group consisting of prostate, lung, bladder, colorectal, and multiple myeloma.

53. The method of claim 50, wherein the cancer is non-small cell lung carcinoma, squamous cell carcinoma, leukemia, acute myelogenous leukemia, chronic myelogenous leukemia, lymphoma, NPM/ALK-transformed anaplastic large cell lymphoma, diffuse large B cell lymphoma, neuroendocrine tumors, breast cancer, mantle cell lymphoma, renal cell carcinoma, rhabdomyosarcoma, ovarian cancer, endometrial cancer, small cell carcinoma, adenocarcinoma, gastric carcinoma, hepatocellular carcinoma, pancreatic cancer, thyroid carcinoma, anaplastic large cell lymphoma, hemangioma, or head and neck cancer.

54. The method of claim 50, wherein the cancer is a solid tumor.

55. The method of claim 50, wherein the cancer is head and neck cancer, squamous cell carcinoma, gastric carcinoma, or pancreatic cancer.

56. A method for treating an autoimmune disease in a subject comprising administering to the subject a therapeutically effective amount of the compound or salt of any one of claims 1 to 40.

57. The method of claim 56, wherein the autoimmune disease is psoriasis, dermatitis, systemic scleroderma, sclerosis, Crohn's disease, ulcerative colitis; respiratory distress syndrome, meningitis; encephalitis; uveitis; colitis; glomerulonephritis; eczema, asthma, chronic inflammation; atherosclerosis; leukocyte adhesion deficiency; rheumatoid arthritis; systemic lupus erythematosus (SLE); diabetes mellitus; multiple sclerosis; Reynaud's syndrome; autoimmune thyroiditis; allergic encephalomyelitis; Sjorgen's syndrome; juvenile onset diabetes; tuberculosis, sarcoidosis, polymyositis, granulomatosis and vasculitis; pernicious anemia (Addison's disease); diseases involving leukocyte diapedesis; central nervous system (CNS) inflammatory disorder; multiple organ injury syndrome; hemolytic anemia; myasthenia gravis; antigen-antibody complex mediated diseases; anti-glomerular basement membrane disease; antiphospholipid syndrome; allergic neuritis; Graves' disease; Lambert-Eaton myasthenic syndrome; pemphigoid bullous; pemphigus; autoimmune polyendocrinopathies; Reiter's disease; stiff-man syndrome; Behcet disease; giant cell arteritis; immune complex nephritis; IgA nephropathy; IgM polyneuropathies; immune thrombocytopenic purpura (ITP) or autoimmune thrombocytopenia.

58. A method for the treatment of an immune-related disease in a subject comprising administering to the subject a therapeutically effective amount of the compound or salt of any one of claims 1 to 40.

59. The method of claim 58, wherein the immune-related disease is rheumatoid arthritis, lupus, inflammatory bowel disease, multiple sclerosis, or Crohn's disease.

60. A method for treating neurodegenerative disease in a subject comprising administering to the subject a therapeutically effective amount of the compound or salt of any one of claims 1 to 40.

61. The method of claim 60, wherein the neurodegenerative disease is multiple sclerosis.

62. A method for treating an inflammatory disease in a subject comprising administering to the subject a therapeutically effective amount of the compound or salt of any one of claims 1 to 40.

63. The method of claim 62, wherein the inflammatory disease is bronchitis, conjunctivitis, myocarditis, pancreatitis, chronic cholecstitis, bronchiectasis, aortic valve stenosis, restenosis, psoriasis or arthritis.
